(19) United States
(12) Patent Application Publication
(10) Pub. No.: US 2025/0230253 A1
(43) Pub. Date: Jul. 17, 2025

(54) ANTI-CD40 ANTIBODIES AND USES THEREOF

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US)

(72) Inventor: Christopher S. HACKETT, New York, NY (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 19/097,798

(22) Filed: Apr. 1, 2025

Related U.S. Application Data
(63) Continuation of application No. PCT/US2023/075805, filed on Oct. 3, 2023.
(60) Provisional application No. 63/412,957, filed on Oct. 4, 2022.

(51) Int. Cl. C07K 16/28; A61K 39/00; A61P 35/00

(52) U.S. Cl. CPC C07K 16/2878; A61P 35/00; A61K 2039/505; C07K 2317/33; C07K 2317/52; C07K 2317/622; C07K 2317/75

(57) ABSTRACT

The presently disclosed subject matter provides anti-CD40 antibodies or antigen-binding fragments thereof, anti-CD40 Fc fusion proteins (e.g., scFv-Fc fusion proteins binding to CD40), cells comprising the anti-CD antibodies or antigen-binding fragments thereof, cells comprising the anti-CD40 Fc fusion proteins, compositions comprising such cells, and methods of using such cells and compositions for diagnosis and therapies.

### CROSS-REFERENCE TO RELATED APPLICATION

This application is a continuation application of International Patent Application No. PCT/US2023/075805, filed Oct. 3, 2023, which claims priority to U.S. Provisional Application No. 63/412,957, filed on Oct. 4, 2022, the contents of each of which are hereby incorporated by reference in their entireties, and to each of which priority is claimed.

### SEQUENCE LISTING

A Sequence Listing conforming to the rules of WIPO Standard ST.26 is hereby incorporated by reference. Said Sequence Listing has been filed as an electronic document via PatentCenter. The electronic document, created on Mar. 31, 2025, is entitled “0727341752.xml”, and is 211,403 bytes in size.

### 1. FIELD OF THE INVENTION

The presently disclosed subject matter relates to anti-CD40 antibodies or antigen-binding fragments thereof, anti-CD40 Fc fusion proteins (e.g., scFv-Fc fusion proteins binding to CD40), cells comprising the anti-CD antibodies or antigen-binding fragments thereof, cells comprising the anti-CD40 Fc fusion proteins, compositions comprising such cells, and methods of using such cells and compositions for diagnosis and therapies.

### 2. BACKGROUND OF THE INVENTION

Immunotherapy with immune checkpoint blockade (anti-CTLA4, anti-PD-1/PD-L1) has had a major impact on cancer treatment. Despite this success, the majority of cancer patients (over 80%) still do not benefit from it. A major hurdle to overcome resistance to immune checkpoint blockade is proper activation of antigen presenting cells (APCs), particularly dendritic cells (DCs), which are responsible for priming T cells. Stimulation of CD40 on APCs has been recognized as a highly effective way to activate APCs. Indeed, CD40 agonists have demonstrated antitumor activity as monotherapy in human cancers (de Vos et al., J Hematol Oncol (2014); 7:44; Vonderheide et al., J Clin Oncol (2007); 25:876-883; Vonderheide et al., J Clin Oncol (2001); 19:3280-3287). And in studies performed in mice, it has been shown that a CD40 agonist antibody can be used to overcome resistance to PD-1 blockade and that combining CD40 activation with TLR4 stimulation using monophosphoryl lipid A (MPL) can restore sensitivity to PD-1 blockade, confer persistent antitumor immunity, and overcome the challenge of T-cell exhaustion in tumors (Khalil et al., J Clin Invest (2019); 130).

Given the significant role for CD40 in activation of APCs in diseases (e.g., cancers), antibodies that bind to CD40 and methods of using such antibodies, are desired.

### 3. SUMMARY OF THE INVENTION

The presently disclosed subject matter provides anti-CD40 antibodies or antigen-binding fragments thereof, anti-CD40 Fc fusion proteins (e.g., scFv-Fc fusion proteins binding to CD40), cells comprising the anti-CD40 antibodies or antigen-binding fragments thereof, cells comprising the anti-CD40 Fc fusion proteins, compositions comprising such cells, and methods of using such cells and compositions for diagnosis and therapies.

In certain embodiments, the anti-CD40 antibody or antigen-binding fragment thereof comprises a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93.

In certain embodiments, the anti-CD40 antibody or antigen-binding fragment thereof comprises a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

In certain embodiments, the anti-CD40 antibody or antigen-binding fragment thereof comprises:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93; and
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

In certain embodiments, the anti-CD40 antibody or antigen-binding fragment thereof comprises a heavy chain variable region and a light chain variable region, wherein the heavy chain variable region and the light chain variable region are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 14, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 25;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 34, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 41, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 49, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 50;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 59, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 60;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 68, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 69;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 78, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 79;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 83, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 84; and
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 93, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 94.

In certain embodiments, the anti-CD40 antibody or antigen-binding fragment thereof comprises a heavy chain variable region comprising the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93. In certain embodiments, the anti-CD40 antibody or antigen-binding fragment thereof comprises a light chain variable region comprising the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

In certain embodiments, the anti-CD40 antibody or antigen-binding fragment thereof comprises:
(a) a heavy chain variable region comprising the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93; or
(b) a light chain variable region comprising the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

In certain embodiments,
(a) the heavy chain variable region comprises the amino acid sequence set forth in SEQ ID NO: 14, and the light chain variable region comprises the amino acid sequence set forth in SEQ ID NO: 15;
(b) the heavy chain variable region comprises the amino acid sequence set forth in SEQ ID NO: 24, and the light chain variable region comprises the amino acid sequence set forth in SEQ ID NO: 25;
(c) the heavy chain variable region comprises the amino acid sequence set forth in SEQ ID NO: 34, and the light chain variable region comprises the amino acid sequence set forth in SEQ ID NO: 35;
(d) the heavy chain variable region comprises the amino acid sequence set forth in SEQ ID NO: 41, and the light chain variable region comprises the amino acid sequence set forth in SEQ ID NO: 35;
(e) the heavy chain variable region comprises the amino acid sequence set forth in SEQ ID NO: 49, and the light chain variable region comprises the amino acid sequence set forth in SEQ ID NO: 50;
(f) the heavy chain variable region comprises the amino acid sequence set forth in SEQ ID NO: 59, and the light chain variable region comprises the amino acid sequence set forth in SEQ ID NO: 60;
(g) the heavy chain variable region comprises the amino acid sequence set forth in SEQ ID NO: 68, and the light chain variable region comprises the amino acid sequence set forth in SEQ ID NO: 69;
(h) the heavy chain variable region comprises the amino acid sequence set forth in SEQ ID NO: 78, and the light chain variable region comprises the amino acid sequence set forth in SEQ ID NO: 79;
(i) the heavy chain variable region comprises the amino acid sequence set forth in SEQ ID NO: 83, and the light chain variable region comprises the amino acid sequence set forth in SEQ ID NO: 84; or
j) the heavy chain variable region comprises the amino acid sequence set forth in SEQ ID NO: 93, and the light chain variable region comprises the amino acid sequence set forth in SEQ ID NO: 94.

In certain embodiments,
(a) the heavy chain variable region comprises the amino acid sequence set forth in SEQ ID NO: 14, and the light chain variable region comprises the amino acid sequence set forth in SEQ ID NO: 15; or
(b) the heavy chain variable region comprises the amino acid sequence set forth in SEQ ID NO: 24, and the light chain variable region comprises the amino acid sequence set forth in SEQ ID NO: 25.

In certain embodiments, the anti-CD40 antibody or antigen-binding fragment thereof comprises a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
(a) a heavy chain variable region CDR3 comprising the amino acid sequence set forth in SEQ ID NO: 10 or a conservative modification thereof; and a light chain variable region CDR3 comprising the amino acid sequence set forth in SEQ ID NO: 13 or a conservative modification thereof;
(b) a heavy chain variable region CDR3 comprising the amino acid sequence set forth in SEQ ID NO: 20 or a conservative modification thereof; and a light chain variable region CDR3 comprising the amino acid sequence set forth in SEQ ID NO: 23 or a conservative modification thereof;
(c) a heavy chain variable region CDR3 comprising the amino acid sequence set forth in SEQ ID NO: 30 or a conservative modification thereof; and a light chain variable region CDR3 comprising the amino acid sequence set forth in SEQ ID NO: 33 or a conservative modification thereof;
(d) a heavy chain variable region CDR3 comprising the amino acid sequence set forth in SEQ ID NO: 40 or a conservative modification thereof; and a light chain variable region CDR3 comprising the amino acid sequence set forth in SEQ ID NO: 33 or a conservative modification thereof;
(e) a heavy chain variable region CDR3 comprising the amino acid sequence set forth in SEQ ID NO: 45 or a conservative modification thereof; and a light chain variable region CDR3 comprising the amino acid sequence set forth in SEQ ID NO: 48 or a conservative modification thereof;
(f) a heavy chain variable region CDR3 comprising the amino acid sequence set forth in SEQ ID NO: 55 or a conservative modification thereof; and a light chain variable region CDR3 comprising the amino acid sequence set forth in SEQ ID NO: 58 or a conservative modification thereof;
(g) a heavy chain variable region CDR3 comprising the amino acid sequence set forth in SEQ ID NO: 65 or a conservative modification thereof; and a light chain variable region CDR3 comprising the amino acid sequence set forth in SEQ ID NO: 67 or a conservative modification thereof;
(h) a heavy chain variable region CDR3 comprising the amino acid sequence set forth in SEQ ID NO: 74 or a conservative modification thereof; and a light chain variable region CDR3 comprising the amino acid sequence set forth in SEQ ID NO: 77 or a conservative modification thereof;
(i) a heavy chain variable region CDR3 comprising the amino acid sequence set forth in SEQ ID NO: 82 or a conservative modification thereof; and a light chain variable region CDR3 comprising the amino acid sequence set forth in SEQ ID NO: 67 or a conservative modification thereof; or
(j) a heavy chain variable region CDR3 comprising the amino acid sequence set forth in SEQ ID NO: 89 or a conservative modification thereof; and a light chain variable region CDR3 comprising the amino acid sequence set forth in SEQ ID NO: 92 or a conservative modification thereof.

In certain embodiments, the heavy chain variable region and light chain variable region CDR2 domains are selected from the group consisting of:
(a) a heavy chain variable region CDR2 comprising the amino acid sequence set forth in SEQ ID NO: 9 or a conservative modification thereof; and a light chain variable region CDR2 comprising the amino acid sequence set forth in SEQ ID NO: 12 or a conservative modification thereof;
(b) a heavy chain variable region CDR2 comprising the amino acid sequence set forth in SEQ ID NO: 19 or a conservative modification thereof; and a light chain variable region CDR2 comprising the amino acid sequence set forth in SEQ ID NO: 22 or a conservative modification thereof;
(c) a heavy chain variable region CDR2 comprising the amino acid sequence set forth in SEQ ID NO: 29 or a conservative modification thereof; and a light chain variable region CDR2 comprising the amino acid sequence set forth in SEQ ID NO: 32 or a conservative modification thereof;
(d) a heavy chain variable region CDR2 comprising the amino acid sequence set forth in SEQ ID NO: 39 or a conservative modification thereof; and a light chain variable region CDR2 comprising the amino acid sequence set forth in SEQ ID NO: 32 or a conservative modification thereof;
(e) a heavy chain variable region CDR2 comprising the amino acid sequence set forth in SEQ ID NO: 44 or a conservative modification thereof; and a light chain variable region CDR2 comprising the amino acid sequence set forth in SEQ ID NO: 47 or a conservative modification thereof;
(f) a heavy chain variable region CDR2 comprising the amino acid sequence set forth in SEQ ID NO: 54 or a conservative modification thereof; and a light chain variable region CDR2 comprising the amino acid sequence set forth in SEQ ID NO: 57 or a conservative modification thereof;
(g) a heavy chain variable region CDR2 comprising the amino acid sequence set forth in SEQ ID NO: 64 or a conservative modification thereof; and a light chain variable region CDR2 comprising the amino acid sequence set forth in SEQ ID NO: 22 or a conservative modification thereof;
(h) a heavy chain variable region CDR2 comprising the amino acid sequence set forth in SEQ ID NO: 73 or a conservative modification thereof; and a light chain variable region CDR2 comprising the amino acid sequence set forth in SEQ ID NO: 76 or a conservative modification thereof;
(i) a heavy chain variable region CDR2 comprising the amino acid sequence set forth in SEQ ID NO: 64 or a conservative modification thereof; and a light chain variable region CDR2 comprising the amino acid sequence set forth in SEQ ID NO: 22 or a conservative modification thereof; and
(j) a heavy chain variable region CDR2 comprising the amino acid sequence set forth in SEQ ID NO: 88 or a conservative modification thereof; and a light chain variable region CDR2 comprising the amino acid sequence set forth in SEQ ID NO: 91 or a conservative modification thereof.

In certain embodiments, the heavy chain variable region and light chain variable region CDR1 domains are selected from the group consisting of:
(a) a heavy chain variable region CDR1 comprising the amino acid sequence set forth in SEQ ID NO: 8 or a conservative modification thereof; and a light chain variable region CDR1 comprising the amino acid sequence set forth in SEQ ID NO: 11 or a conservative modification thereof;
(b) a heavy chain variable region CDR1 comprising the amino acid sequence set forth in SEQ ID NO: 18 or a conservative modification thereof; and a light chain variable region CDR1 comprising the amino acid sequence set forth in SEQ ID NO: 21 or a conservative modification thereof;
(c) a heavy chain variable region CDR1 comprising the amino acid sequence set forth in SEQ ID NO: 28 or a conservative modification thereof; and a light chain variable region CDR1 comprising the amino acid sequence set forth in SEQ ID NO: 31 or a conservative modification thereof;
(d) a heavy chain variable region CDR1 comprising the amino acid sequence set forth in SEQ ID NO: 38 or a conservative modification thereof; and a light chain variable region CDR1 comprising the amino acid sequence set forth in SEQ ID NO: 31 or a conservative modification thereof;
(e) a heavy chain variable region CDR1 comprising the amino acid sequence set forth in SEQ ID NO: 43 or a conservative modification thereof; and a light chain variable region CDR1 comprising the amino acid sequence set forth in SEQ ID NO: 46 or a conservative modification thereof;
(f) a heavy chain variable region CDR1 comprising the amino acid sequence set forth in SEQ ID NO: 53 or a conservative modification thereof; and a light chain variable region CDR1 comprising the amino acid sequence set forth in SEQ ID NO: 56 or a conservative modification thereof;
(g) a heavy chain variable region CDR1 comprising the amino acid sequence set forth in SEQ ID NO: 63 or a conservative modification thereof; and a light chain variable region CDR1 comprising the amino acid sequence set forth in SEQ ID NO: 66 or a conservative modification thereof;
(h) a heavy chain variable region CDR1 comprising the amino acid sequence set forth in SEQ ID NO: 72 or a conservative modification thereof; and a light chain variable region CDR1 comprising the amino acid sequence set forth in SEQ ID NO: 75 or a conservative modification thereof;
(i) a heavy chain variable region CDR1 comprising the amino acid sequence set forth in SEQ ID NO: 63 or a conservative modification thereof; and a light chain variable region CDR1 comprising the amino acid sequence set forth in SEQ ID NO: 66 or a conservative modification thereof; or
(j) a heavy chain variable region CDR1 comprising the amino acid sequence set forth in SEQ ID NO: 87 or a conservative modification thereof; and a light chain variable region CDR1 comprising the amino acid sequence set forth in SEQ ID NO: 90 or a conservative modification thereof.

In certain embodiments, one or more of the CDR sequences have up to about 5 amino acid substitutions. In certain embodiments, one or more of the CDR sequences have up to about 3 amino acid substitutions.

In certain embodiments, the anti-CD40 antibody or antigen-binding fragment thereof comprises:
(a) a heavy chain variable region comprising a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
(b) a heavy chain variable region comprising a CDR1 comprising the amino acid sequence set forth in SEQ ID NO: 18, a CDR2 comprising the amino acid sequence set forth in SEQ ID NO: 19, and a CDR3 comprising the amino acid sequence set forth in SEQ ID NO: 20;
(c) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30;
(d) a heavy chain variable region comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40;
(e) a heavy chain variable region comprising a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45;
(f) a heavy chain variable region comprising a CDR1 comprising the amino acid sequence set forth in SEQ ID NO: 53, a CDR2 comprising the amino acid sequence set forth in SEQ ID NO: 54, and a CDR3 comprising the amino acid sequence set forth in SEQ ID NO: 55;
(g) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65;
(h) a heavy chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
(i) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 82; or
(j) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89.

In certain embodiments, the anti-CD40 antibody or an antigen-binding fragment thereof comprises:
(a) a light chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
(b) a light chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23;
(c) a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(d) a light chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
(e) a light chain variable region comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58;
(f) a light chain variable region comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67;
(g) a light chain variable region comprising a CDR1 comprising the amino acid sequence set forth in SEQ ID NO: 75, a CDR2 comprising the amino acid sequence set forth in SEQ ID NO: 76, and a CDR3 comprising the amino acid sequence set forth in SEQ ID NO: 77; or
(h) a light chain variable region comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92.

In certain embodiments, the anti-CD40 antibody or antigen-binding fragment thereof comprises:
(a) a heavy chain variable region comprising a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and a light chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
(b) a heavy chain variable region comprising a CDR1 comprising the amino acid sequence set forth in SEQ ID NO: 18, a CDR2 comprising an amino acid sequence set forth in SEQ ID NO: 19, and a CDR3 comprising the amino acid sequence set forth in SEQ ID NO: 20; and a light chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23;
(c) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(d) a heavy chain variable region comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(e) a heavy chain variable region comprising a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and a light chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
(f) a heavy chain variable region comprising a CDR1 comprising the amino acid sequence set forth in SEQ ID NO: 53, a CDR2 comprising the amino acid sequence set forth in SEQ ID NO: 54, and a CDR3 comprising the amino acid sequence set forth in SEQ ID NO: 55; and a light chain variable region comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58;
(g) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65; and a light chain variable region comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67;
(h) a heavy chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and a light chain variable region comprising a CDR1 comprising the amino acid sequence set forth in SEQ ID NO: 75, a CDR2 comprising the amino acid sequence set forth in SEQ ID NO: 76, and a CDR3 comprising the amino acid sequence set forth in SEQ ID NO: 77;
(i) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 82; and a light chain variable region comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67; or
(j) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92.

In certain embodiments, the anti-CD40 antibody or antigen-binding fragment thereof comprises: a heavy chain variable region comprising a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and a light chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13.

In certain embodiments, the anti-CD40 antibody or antigen-binding fragment thereof comprises: a heavy chain variable region comprising a CDR1 comprising the amino acid sequence set forth in SEQ ID NO: 18, a CDR2 comprising an amino acid sequence set forth in SEQ ID NO: 19, and a CDR3 comprising the amino acid sequence set forth in SEQ ID NO: 20; and a light chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23.

In certain embodiments, the anti-CD40 antibody or antigen-binding fragment thereof binds to a CD40 comprising the amino acid sequence set forth in SEQ ID NO: 7 or a fragment thereof. In certain embodiments, the anti-CD40 antibody or antigen-binding fragment thereof comprises a human variable region framework region. In certain embodiments, the anti-CD40 antibody or antigen-binding fragment thereof is a fully human or an antigen-binding fragment thereof. In certain embodiments, the anti-CD40 antibody or antigen-binding fragment thereof is a chimeric antibody or an antigen-binding fragment thereof. In certain embodiments, the anti-CD40 antibody or antigen-binding fragment thereof is a humanized antibody or an antigen-binding fragment thereof. In certain embodiments, the anti-CD40 antibody or antigen-binding fragment thereof is a murine antibody or an antigen-binding fragment thereof. In certain embodiments, the anti-CD40 antibody or an antigen-binding fragment thereof is a Fab, Fab′, F(ab′)₂, variable fragment (Fv), or single chain variable region (scFv). In certain embodiments, the anti-CD40 antibody or antigen-binding fragment thereof is an scFv.

The presently disclosed subject matter also provides antibodies or antigen-binding fragments thereof, which cross-compete for binding to CD40 with any one of the anti-CD40 antibodies or antigen-binding fragments thereof disclosed herein. Furthermore, the presently disclosed subject matter provides antibodies or antigen-binding fragments thereof, which bind to the same epitope region on CD40 with any one of the anti-CD40 antibodies or antigen-binding fragments thereof disclosed herein.

The presently disclosed subject matter provides compositions comprising the anti-CD40 antibodies or antigen-binding fragments thereof disclosed herein. In certain embodiments, the composition is a pharmaceutical composition that further comprises a pharmaceutically acceptable carrier. Furthermore, the presently disclosed subject matter provides immunoconjugates comprising the antibodies or antigen-binding fragments thereof disclosed herein, linked to a therapeutic agent. In certain embodiments, the therapeutic agent is a drug, a radioactive isotope.

The presently disclosed subject matter also provides compositions comprising the immunoconjugates disclosed herein. In certain embodiments, the composition is a pharmaceutical composition that further comprises a pharmaceutically acceptable carrier.

The presently disclosed subject matter provides multi-specific molecules comprising the anti-CD40 antibodies or antigen-binding fragments thereof disclosed herein, linked to one or more functional moieties. In certain embodiments, the one or more functional moieties have a different binding specificity than the antibody or antigen binding fragment thereof. Furthermore, the presently disclosed subject matter provides compositions comprising the multi-specific molecules disclosed herein. In certain embodiments, the composition is a pharmaceutical composition that further comprises a pharmaceutically acceptable carrier.

The presently disclosed subject matter provides Fc fusion proteins comprising the anti-CD40 antibodies or antigen-binding fragments thereof disclosed herein. In certain embodiments, the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 122 or SEQ ID NO: 124. In certain embodiments, the Fc fusion protein comprises the amino acid sequence set forth in SEQ ID NO: 122 or SEQ ID NO: 124. Furthermore, the presently disclosed subject matter provides compositions comprising the Fc fusion proteins disclosed herein. In certain embodiments, the composition is a pharmaceutical composition that further comprises a pharmaceutically acceptable carrier.

The presently disclosed subject matter provides nucleic acids that encode the anti-CD40 antibodies or antigen-binding fragments thereof disclosed herein. The presently disclosed subject matter also provides nucleic acids that encode the Fc fusion proteins disclosed herein. The presently disclosed subject matter provides vectors comprising the nucleic acids disclosed herein. The presently disclosed subject matter further provides host cells comprising the vectors disclosed herein.

Additionally, the presently disclosed subject matter provides immunoresponsive cells comprising the anti-CD40 antibodies or antigen-binding fragments thereof, the immunoconjugate, the multi-specific molecule, the Fc fusion protein, the nucleic acid, or the vector disclosed herein.

In certain embodiments, the immunoresponsive cell further comprises antigen-recognizing receptor. In certain embodiments, the antigen-recognizing receptor is a recombinant T cell receptor (TCR), a chimeric antigen receptor (CAR), or a TCR like fusion molecule. In certain embodiments, the antigen-recognizing receptor is a CAR. In certain embodiments, the antigen is a tumor antigen or a pathogen antigen. In certain embodiments, the antigen is a tumor antigen. In certain embodiments, the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, ERBB, Tyrosinase related protein 1 (TYRP1, gp75), and Delta-like 3 (DLL3).

In certain embodiments, the immunoresponsive cell is a cell of the lymphoid lineage or a cell of the myeloid lineage. In certain embodiments, the cell is selected from the group consisting of a T cell, a Natural Killer (NK) cell, a B cell, a monocyte, and a macrophage, a pluripotent stem cell from which a lymphoid cell may be differentiated, a pluripotent stem cell from which a myeloid cell may be differentiated, and combinations thereof. In certain embodiments, the cell is a T cell. In certain embodiments, the cell is a Natural Killer (NK) cell. In certain embodiments, the cell is autologous. In certain embodiments, the cell is allogeneic.

The presently disclosed subject matter also provides compositions comprising the immunoresponsive cells disclosed herein. In certain embodiments, the composition is a pharmaceutical composition further comprising a pharmaceutically acceptable excipient.

The presently disclosed subject matter provides methods for detecting CD40 in a whole cell, a tissue, or a blood sample. In certain embodiments, the method comprises contacting a cell, tissue or blood sample with the anti-CD40 antibody or antigen-binding fragment thereof disclosed herein, wherein the anti-CD40 antibody or antigen-binding fragment thereof comprises a detectable label. In certain embodiments, the method comprises determining the amount of the labeled anti-CD40 antibody or antigen-binding fragment thereof bound to the cell, tissue or blood sample by measuring the amount of detectable label associated with said cell or tissue, wherein the amount of bound anti-CD40 antibody or antigen-binding fragment thereof indicates the amount of CD40 in the cell, tissue or blood sample.

The presently disclosed subject matter further provides methods of preventing and/or treating a disease or disorder in a subject. In certain embodiments, the method comprises administering to the subject the anti-CD40 antibodies or antigen-binding fragments thereof, the immunoconjugates, the multi-specific molecules, the Fc fusion proteins, the cells, or the compositions disclosed herein. In certain embodiments, the disease or disorder is a tumor. In certain embodiments, the disease or disorder is a pathogen infection. In certain embodiments, the disease or disorder is an autoimmune disease. In certain embodiments, the disease or disorder is an infectious disease.

In certain embodiments, the tumor is cancer. In certain embodiments, the tumor is hematological cancer or solid tissue cancer. In certain embodiments, the cancer is selected from the group consisting of Hodgkin lymphoma, non-Hodgkin's lymphoma, B-cell lymphomas, acute myelogenous leukemia, chronic myelogenous leukemia, acute lymphoblastic leukemia, chronic lymphocytic leukemia, hairy cell leukemia, multiple myeloma, melanoma, small cell lung cancer, sarcoma, glioma, neuroblastoma, prostate cancer, breast cancer, lung cancer, pancreatic cancer, gastric cancer, esophageal cancer, colon cancer, rectal cancer, bladder cancer, renal cell carcinoma, hepatocellular carcinoma, cholangiocarcinoma, Merkel cell carcinoma, head and neck cancer, thyroid cancer, salivary gland cancer, ovarian cancer, endometrial cancer, cervical cancer, germ cell tumor, testicular cancer, basal cell carcinoma, squamous cell carcinoma, mesothelioma, retinoblastoma, neurofibroma, pheochromocytoma, and adrenal gland tumor. In certain embodiments, the subject is a human.

The presently disclosed subject matter provides kits for preventing and/or treating a disease or disorder in a subject, comprising the anti-CD40 antibodies or antigen-binding fragments thereof, the immunoconjugates, the multi-specific molecules, the Fc fusion proteins, the cells, or the compositions disclosed herein. In certain embodiments, the kit further comprises written instructions for using the anti-CD40 antibodies or antigen-binding fragments thereof, immunoconjugates, multi-specific molecules, Fc fusion proteins, cells, or compositions disclosed herein.

### 4. BRIEF DESCRIPTION OF THE FIGURES

The following Detailed Description, given by way of example, but not intended to limit the invention to specific embodiments described, may be understood in conjunction with the accompanying drawings.

FIGS. 1A-1C depict candidate antibodies binding to CD40. Binding of ten (10) selected clones tested by ELISA on mouse (mCD40) and human (huCD40) proteins. All 10 clonal monoclonal (mAbs) showed binding to human CD40 but not to mouse CD40. Anti-His, anti-CD40 and irrelevant protein with His were used as controls. FIG. 1A shows the ELISA signal of clones post-thaw. FIG. 1B shows the ELISA signal of different clones of the screened hybridomas. FIG. 1C shows the ELISA signal of clone A supernatants.

### 5. DETAILED DESCRIPTION OF EMBODIMENTS OF THE INVENTION

The presently disclosed subject matter provides anti-CD40 antibodies or antigen-binding fragments thereof, anti-CD40 Fc fusion proteins (e.g., scFv-Fc fusion proteins binding to CD40), cells comprising the anti-CD40 antibodies or antigen-binding fragments thereof, cells comprising the anti-CD40 Fc fusion proteins, compositions comprising such cells, and methods of using such cells and compositions for diagnosis and therapies. Non-limiting embodiments of the present disclosure are described by the present specification and Examples.

For purposes of clarity of disclosure and not by way of limitation, the detailed description is divided into the following subsections:
5.1. Definitions;
5.2. CD40;
5.3. Anti-CD40 Antibodies or Antigen-Binding Fragments thereof;
5.4. Fc Fusion Proteins;
5.5. Nucleic Acids encoding the Antibodies, Antigen-binding Fragments, and/or Fc Fusion Proteins;
5.6. Cells;
5.7. Pharmaceutical Compositions and Methods of Treatment;
5.8. Diagnostic and Prognostic Methods;
5.9. Kits; and
5.10. Exemplary Embodiments.

### 5.1. Definitions

In the description that follows, certain conventions will be followed as regards the usage of terminology. Generally, terms used herein are intended to be interpreted consistently with the meaning of those terms as they are known to those of skill in the art.

“Antibody” and “antibodies” as those terms are known in the art refer to antigen binding proteins of the immune system. The term “antibody” as referred to herein includes whole, full length antibodies having an antigen-binding region, and any fragment thereof in which the “antigen-binding fragment” or “antigen-binding region” is retained, or single chains, for example, single chain variable fragment (scFv), thereof. A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (CH) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the presently disclosed subject matter may include amino acid residues not encoded by human germline immunoglobulin sequences (e.g., mutations introduced by random or site-specific mutagenesis in vitro or by somatic mutation in vivo).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to CD40” is intended to refer to an antibody that binds to CD40 (e.g., human CD40, e.g., soluble human CD40) with a dissociation constant (K_{D}) of about 1×10⁻⁸ M or less, about 5×10⁻⁹ M or less, about 1×10⁻⁹ M or less, about 5×10⁻¹ M or less, about 1×10⁻¹⁰ M or less, about 5×10⁻¹¹ M or less, or about 1×10⁻¹¹ M or less.

An “antibody that competes for binding” or “antibody that cross-competes for binding” with a reference antibody for binding to an antigen, e.g., CD40, refers to an antibody that blocks binding of the reference antibody to the antigen (e.g., CD40) in a competition assay by 50% or more, and conversely, the reference antibody blocks binding of the antibody to the antigen (e.g., CD40) in a competition assay by 50% or more. An exemplary competition assay is described in “Antibodies,” Harlow and Lane (Cold Spring Harbor Press, Cold Spring Harbor, NY).

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen (e.g., a CD40 polypeptide).”

The term “antigen-binding fragment” or “antigen-binding region” of an antibody, as used herein, refers to that region or fragment of the antibody that binds to the antigen and which confers antigen specificity to the antibody; fragments of antigen-binding proteins, for example, antibodies includes one or more fragments of an antibody that retain the ability to specifically bind to an antigen (e.g., a CD40 polypeptide). It has been shown that the antigen-binding function of an antibody can be performed by fragments of a full-length antibody. Examples of antigen-binding fragments encompassed within the term “antibody fragments” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 1989; 341:544-546), which consists of a V_{H} domain; and an isolated complementarity determining region (CDR).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science (1988); 242:423-426; and Huston et al., Proc Natl Acad Sci (1998); 85:5879-5883. These antibody fragments are obtained using conventional techniques known to those of skill in the art, and the fragments are screened for utility in the same manner as are intact antibodies.

An “antibody” or “antigen-binding protein” is one which has been identified and separated and/or recovered from a component of its natural environment. “Synthetic antibodies” or “recombinant antibodies” are generally generated using recombinant technology or using peptide synthetic techniques known to those of skill in the art.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen-binding domain.

Non-limiting examples of linkers are disclosed in Shen et al., Anal Chem (2008); 80(6):1910-1917 and WO 2014/087010, the contents of which are hereby incorporated by reference in their entireties. In certain embodiments, the linker is a G4S linker. In certain embodiments, the linker comprises or consists of the amino acid sequence set forth in SEQ ID NO: 1, which is provided below:
GGGGSGGGGSGGGSGGGGS [SEQ ID NO: 1]

In certain embodiments, the linker comprises or consists of the amino acid sequence set forth in SEQ ID NO: 2, which is provided below:
GGGGSGGGGSGGGGS [SEQ ID NO: 2]

In certain embodiments, the linker comprises or consists of the amino acid sequence set forth in SEQ ID NO: 3, which is provided below:
GGGGSGGGGSGGGGSGGGSGGGGS [SEQ ID NO: 3]

In certain embodiments, the linker comprises or consists of the amino acid sequence set forth in SEQ ID NO: 4, which is provided below:
GGGGSGGGGSGGGGSGGGGSGGGSGGGGS [SEQ ID NO: 4]

In certain embodiments, the linker comprises or consists of the amino acid sequence set forth in SEQ ID NO: 5, which is provided below:
GGGGS [SEQ ID NO: 5]

In certain embodiments, the linker comprises or consists of the amino acid sequence set forth in SEQ ID NO: 6, which is provided below:
GGGGSGGGGS [SEQ ID NO: 6]

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 1988; 85:5879-5883). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008; 27(6):455-51; Peter et al., J Cachexia Sarcopenia Muscle 2012 Aug. 12; Shieh et al., J Imunol 2009; 183(4):2277-85; Giomarelli et al., Thromb Haemost 2007; 97(6):955-63; Fife eta., J Clin Invst 2006; 116(8):2252-61; Brocks et al., Immunotechnology 1997; 3(3):173-84; Moosmayer et al., Ther Immunol 1995; 2(10:31-40). Agonistic scFvs having stimulatory activity have been described (see, e.g., Peter et al., J Bioi Chern 2003; 25278(38):36740-7; Xie et al., Nat Biotech 1997; 15(8):768-71; Ledbetter et al., Crit Rev Immunol 1997; 17(5-6):427-55; Ho et al., BioChim Biophys Acta 2003; 1638(3):257-66).

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

“CDRs” are defined as the complementarity determining region amino acid sequences of an antibody which are the hypervariable regions of immunoglobulin heavy and light chains. See, e. g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise three heavy chain and three light chain CDRs or CDR regions in the variable region. CDRs provide the majority of contact residues for the binding of the antibody to the antigen or epitope region. In certain embodiments, the CDRs are identified according to the IMGT system. In certain embodiments, the CDRs are identified using the IMGT numbering system accessible at www.imgt.org/IMGT_vquest/input.

The terms “isolated” denotes a degree of separation from original source or surroundings.

An “isolated antibody” is one which has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr (2007); B 848:79-87.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

An “isolated nucleic acid encoding an antibody” (including references to a specific antibody, e.g. an anti-CD40 antibody) refers to one or more nucleic acid molecules encoding antibody heavy and light chains (or fragments thereof), including such nucleic acid molecule(s) in a single vector separate vectors, and such nucleic acid molecule(s) present at one or more locations in a host cell.

An “immunoconjugate” is an antibody conjugated to one or more heterologous molecule(s), including, but not limited to, a cytotoxic agent.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition, and the form and effective concentration of the cells administered.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing recurrence of disease, alleviation of symptoms, diminishment of any direct or indirect pathological consequences of the disease, preventing metastasis, decreasing the rate of disease progression, amelioration or palliation of the disease state, and remission or improved prognosis. In certain embodiments, antibodies of the presently disclosed subject matter are used to delay development of a disease or to slow the progression of a disease, e.g., a tumor.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 chains cluster in response to ligand binding and immunoreceptor tyrosine-based inhibition motifs (ITAMs) a signal transduction cascade is produced. In certain embodiments, when a CAR or a TCR like fusion molecule binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen (e.g., a cancer antigen). In certain embodiments, the antigen-recognizing receptor is capable of activating an immune or immunoresponsive cell (e.g., a T cell) upon its binding to the target antigen.

The term “chimeric antigen receptor” or “CAR” as used herein refers to a molecule comprising an extracellular antigen-binding domain that is fused to an intracellular signaling domain that is capable of activating or stimulating an immunoresponsive cell, and a transmembrane domain. In certain embodiments, the extracellular antigen-binding domain of a CAR comprises a scFv. The scFv can be derived from fusing the variable heavy and light regions of an antibody. In certain embodiments, the scFv may be derived from Fab's (instead of from an antibody, e.g., obtained from Fab libraries). In certain embodiments, the scFv is fused to the transmembrane domain and then to the intracellular signaling domain.

By “substantially identical” or “substantially homologous” is meant a polypeptide or nucleic acid molecule exhibiting at least about 50% homologous or identical to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

As used herein, the term “immunosuppressive activity” is meant induction of signal transduction or changes in protein expression in a cell (e.g., an activated immunoresponsive cell) resulting in a decrease in an immune response. Non-limiting examples of polypeptides suppressing or decreasing an immune response via their binding include CD47, PD-1, CTLA-4, BTLA, LAG-3, 2B4, and their corresponding ligands (including, but not limited to, SIRPa, PD-L1, PD-L2, TNFRSF14, CD48, and FGL-1). Such polypeptides are present in the tumor microenvironment and inhibit immune responses to neoplastic cells. In certain embodiments, inhibiting, blocking, or antagonizing the interaction of immunosuppressive polypeptides and/or their ligands enhances the immune response of the immunoresponsive cell.

As used herein, the term “immunostimulatory activity” is meant induction of signal transduction or changes in protein expression in a cell (e.g., an activated immunoresponsive cell) resulting in an increase in an immune response. Immunostimulatory activity may include pro-inflammatory activity. Non-limiting examples of polypeptides stimulating or increasing an immune response via their binding include CD28, CD40, OX-40, 4-1BB, GITR, and their corresponding ligands, including B7-1, B7-2, CD40L, OX-40L, 4-1BBL, GITRL. Such polypeptides are present in the tumor microenvironment and activate immune responses to neoplastic cells. In certain embodiments, promoting, stimulating, or agonizing pro-inflammatory polypeptides and/or their ligands enhances the immune response of the immunoresponsive cell.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells). The neoplasia can a primary tumor or primary cancer. In addition, the neoplasm can be in metastatic status.

By “receptor” is meant a polypeptide, or portion thereof, present on a cell membrane that selectively binds one or more ligand.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. In certain embodiments, the signal sequence comprises or consists of the amino acid sequence set forth in SEQ ID NO: 116. An exemplary nucleotide sequence encoding the amino acid sequence of SEQ ID NO: 116 is set forth in SEQ ID NO: 117. SEQ ID NO: 116 and SEQ ID NO: 117 are provided below:

[SEQ ID NO: 116]
METDTLLLWVLLLWVPGSTG
[SEQ ID NO: 117]
ATGGAAACCGATACACTCTTGCTGTGGGTGCTTCTTCTTTGGGTGCCTG
GATCTACTGGC

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The terms “comprises”, “comprising”, and are intended to have the broad meaning ascribed to them in U.S. Patent Law and can mean “includes”, “including” and the like.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As described herein, any concentration range, percentage range, ratio range or integer range is to be understood to include the value of any integer within the recited range and, when appropriate, fractions thereof (such as one tenth and one hundredth of an integer), unless otherwise indicated.

Other aspects of the presently disclosed subject matter are described in the following disclosure and are within the ambit of the presently disclosed subject matter.

### 5.2. CD40

CD40 is a cell-surface member of the TNF (tumor necrosis factor) receptor superfamily. CD40 is expressed broadly in hematopoietic and nonhematopoietic tissues and regulates immunity thus providing a tractable target pathway for cancer immunotherapy (Vonderheide, Annual review of medicine 71 (2020): 47-58). Upon activation, CD40 can license dendritic cells (DCs) to promote antitumor T cell activation and re-educate macrophages to destroy tumor stroma. On DCs, CD40 activation results in two main cellular phenotypes. First, CD40 activation leads to upregulation of major histocompatibility complex (MHC) molecules, increased expression of immunoglobulin (Ig) superfamily costimulatory molecules such as CD86, and upregulation of other tumor necrosis factor (TNF) superfamily ligands such as CD137 ligand, GITR ligand, and OX40 ligand (Vonderheide, Annual review of medicine 71 (2020): 47-58). CD40 is therefore described as proximal in the cascade of adaptive immune activation, receiving a signal from CD40L on CD4⁺ cells, then upregulating on DCs a portfolio of secondary stimulatory molecules that accomplish enhanced antigen presentation and activation of CD8⁺ T cells. A similar change in cell surface phenotype, especially with regard to MHC, CD80, and CD86, occurs in B cells. It is notable that CD40/CD40L is unique in its physical orientation on the DC:T cell synapse, with the TNF receptor (i.e., CD40) being expressed on the DC whereas, for most other TNF receptor/ligand pairs, the ligand is expressed on the DC, reflecting the proximal role of CD40 in the process. Second, CD40-activated DCs elaborate an increased level of critical T cell stimulatory cytokines, including interleukin (IL)-12 p70, which is important for CD8+ T cell activation and skewing of the adaptive immune response toward a Th1 polarization (Vonderheide, Annual review of medicine 71 (2020): 47-58).

In certain embodiments, the presently disclosed anti-CD40 antibodies or antigen-binding fragments thereof bind to human CD40. In certain embodiments, the human CD40 comprises or consists of the amino acid sequence with a UniProt Reference No: P25942-1 (SEQ ID NO: 7) or a fragment thereof. SEQ ID NO: 7 is provided below. In certain embodiments, the human CD40 comprises an extracellular domain, a transmembrane domain, and a cytoplasmic domain. In certain embodiments, the extracellular domain comprises amino acids 21 to 193 of SEQ ID NO: 7. In certain embodiments, the transmembrane domain comprises amino acids 194 to 215 of SEQ ID NO: 7. In certain embodiments, the cytoplasmic domain comprises amino acids 216 to 277 of SEQ ID NO: 7.

[SEQ ID NO: 7]
MVRLPLQCVLWGCLLTAVHPEPPTACREKQYLINSQCCSLCQPGQKLVS
DCTEFTETECLPCGESEFLDTWNRETHCHQHKYCDPNLGLRVQQKGTSE
TDTICTCEEGWHCTSEACESCVLHRSCSPGFGVKQIATGVSDTICEPCP
VGFFSNVSSAFEKCHPWTSCETKDLVVQQAGTNKTDVVCGPQDRLRALV
VIPIIFGILFAILLVLVFIKKVAKKPTNKAPHPKQEPQEINFPDDLPGS
NTAAPVQETLHGCQPVTQEDGKESRISVQERQ

In certain embodiments, the CD40 comprises an amino acid sequence that is at least about 80%, at least about 85%, at least about 90%, at least about 95%, at least about 96%, at least about 97%, at least about 98%, or at least about 99%, at least about 100% identical to the amino acid sequence set forth in SEQ ID NO: 7 or a fragment thereof.

In certain embodiments, the anti-CD40 antibodies or antigen-binding fragments thereof bind to a portion of human CD40. In certain embodiments, the anti-CD40 antibodies or antigen-binding fragments thereof bind to the extracellular domain of CD40. In certain embodiments, the anti-CD40 antibodies or antigen-binding fragments thereof bind to amino acids 21 to 193 of SEQ ID NO: 7.

### 5.3. Anti-CD40 Antibodies

The antibodies of the presently disclosed subject matter are characterized by particular functional features or properties of the antibodies. For example, the antibodies bind specifically to CD40 (e.g., bind to human CD40).

In certain embodiments, a presently disclosed antibody or antigen-binding fragment binds to CD40 (e.g., human CD40) with a binding affinity, for example with a dissociation constant (K_{D}) of 1×10⁻⁸ M or less, e.g., about 1×10⁻⁸ M or less, about 5×10⁻⁹ M or less, about 1×10⁻⁹ M or less, about 5×10⁻¹⁰ M or less, about 1×10⁻¹⁰ M or less, or about 1×10⁻¹¹ M or less.

The heavy and light chains of a presently disclosed antibody or antigen-binding fragment can be full-length (e.g., an antibody can include at least one (e.g., one or two) complete heavy chains, and at least one (e.g., one or two) complete light chains) or can include an antigen-binding fragment (a Fab, F(ab′)₂, variable fragment (Fv), or a single chain variable fragment (“scFv”)). In certain embodiments, the antibody heavy chain constant region is chosen from, e.g., IgG1, IgG2, IgG3, IgG4, IgM, IgA1, IgA2, IgD, and IgE, particularly chosen from, e.g., IgG1, IgG2, IgG3, and IgG4. In certain embodiments, the immunoglobulin isotype is IgG1 (e.g., human IgG1). The choice of antibody isotype can depend on the immune effector function that the antibody is designed to elicit. In certain embodiments, the antibody light chain constant region is chosen from, e.g., kappa or lambda, particularly kappa.

In constructing a recombinant immunoglobulin, appropriate amino acid sequences for constant regions of various immunoglobulin isotypes and methods for the production of a wide array of antibodies are known to those of skill in the art.

### 5.3.1. Single-Chain Variable Fragments (scFvs)

In certain embodiments, the presently disclosed subject matter includes antibodies or antigen-binding fragments thereof that have the scFv sequence fused to one or more constant domains to form an antibody with an Fc region of a human immunoglobulin to yield a bivalent protein, increasing the overall avidity and stability of the antibody. In addition, the Fc portion allows the direct conjugation of other molecules, including but not limited to fluorescent dyes, cytotoxins, radioisotopes etc. to the antibody for example, for use in antigen quantitation studies, to immobilize the antibody for affinity measurements, for targeted delivery of a therapeutic agent, to test for Fc-mediated cytotoxicity using immune effector cells and many other applications.

The results presented here highlight the specificity, sensitivity and utility of the presently disclosed antibodies or antigen-binding fragments in targeting a CD40 polypeptide (e.g., a human CD40 polypeptide).

In certain embodiments, the anti-CD40 scFv comprises a V_{H} comprising the amino acid sequence set forth in SEQ ID NO: 14. An exemplary nucleotide sequence encoding the amino acid sequence of SEQ ID NO: 14 is set forth in SEQ ID NO: 16 or SEQ ID NO: 118. In certain embodiments, the anti-CD40 scFv comprises a V_{L} comprising the amino acid sequence set forth in SEQ ID NO: 15. An exemplary nucleotide sequence encoding the amino acid sequence of SEQ ID NO: 15 is set forth in SEQ ID NO: 17 or SEQ ID NO: 119. SEQ ID NO: 14-17, 118, and 119 are provided in Table 1. In certain embodiments, the scFv is designated as “10-K9”.

In certain embodiments, the anti-CD40 scFv comprises a V_{H} comprising the amino acid sequence set forth in SEQ ID NO: 14 and a V_{L} comprising the amino acid sequence set forth in SEQ ID NO: 15.

In certain embodiments, the anti-CD40 scFv comprises a V_{H} Comprising a CDR1 comprising the amino acid sequence set forth in SEQ ID NO: 8 or a conservative modification thereof, a CDR2 comprising the amino acid sequence set forth in SEQ ID NO: 9 or a conservative modification thereof, and a CDR3 comprising the amino acid sequence set forth in SEQ ID NO: 10 or a conservative modification thereof. SEQ ID NOs: 8-10 are provided in Table 1.

In certain embodiments, the anti-CD40 scFv comprises a V_{L} comprising a CDR1 comprising the amino acid sequence set forth in SEQ ID NO: 11 or a conservative modification thereof, a CDR2 comprising the amino acid sequence set forth in SEQ ID NO: 12 or a conservative modification thereof, and a CDR3 comprising the amino acid sequence set forth in SEQ ID NO: 13 or a conservative modification thereof. SEQ ID NOs: 11-13 are provided in Table 1.

In certain embodiments, the anti-CD40 scFv comprises a V_{H} Comprising a CDR1 comprising the amino acid sequence set forth in SEQ ID NO: 8 or a conservative modification thereof, a CDR2 comprising the amino acid sequence set forth in SEQ ID NO: 9 or a conservative modification thereof, and a CDR3 comprising the amino acid sequence set forth in SEQ ID NO: 10 or a conservative modification thereof; and a V_{L} comprising a CDR1 comprising the amino acid sequence set forth in SEQ ID NO: 11 or a conservative modification thereof, a CDR2 comprising the amino acid sequence set forth in SEQ ID NO: 12 or a conservative modification, and a CDR3 comprising the amino acid sequence set forth in SEQ ID NO: 13 or a conservative modification thereof.

In certain embodiments, the anti-CD40 scFv comprises a V_{H} Comprising a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and a V_{L} comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13.

In certain embodiments, the anti-CD40 scFv comprises a V_{H} comprising the amino acid sequence set forth in SEQ ID NO: 14, and a V_{L} comprising the amino acid sequence set forth in SEQ ID NO: 15. In certain embodiments, the V_{H} and V_{L} are linked via a linker. In certain embodiments, the linker comprises the amino acid sequence set forth in SEQ ID NO: 2.

In certain embodiments, the variable regions are linked one after another such that a heavy chain variable region (V_{H}) is position at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 1
(10-K9)

| CDRs | 1 | 2 | 3 |
|---|---|---|---|
| V_{H} | GFTFSSYG [SEQ ID | ISSDGSHT [SEQ ID | VRQGRYVPSFDV |
| | NO: 8] | NO: 9] | [SEQ ID NO: 10] |
| V_{L} | QSIRNN [SEQ ID NO: | DAS [SEQ ID NO: 12] | HQTNNWPFT |
| | 11] | | [SEQ ID NO: 13] |

| | |
|---|---|
| Full V_{H} | EVQLVESGGDLVKPGGSLKLSCAASGFTFSSYGMSWVRQTPDKRLEWVATISSDGSHTYYPDSVKGRF |
| | TISRDIAKNTLYLQMSSLKSEDTAMYYCVRQGRYVPSFDVWGAGTTVTVSS [SEQ ID NO: 14] |
| Full V_{L} | DIVLTQSPATLSVTPGDSVSLSCRASQSIRNNLHWYQQKSHESPRLLIKDASQSISGTPSRFSGSGSG |
| | TDFTLSINSVETEDFGMYFCHQTNNWPFTFGSGTKLEIK [SEQ ID NO: 15] |
| DNA for | GAGGTGCAGCTGGTGGAGTCTGGGGGAGACTTAGTGAAGCCTGGAGGGTCCCTGAAACTCTCCTGTGC |
| Full V_{H} | AGCCTCTGGATTCACTTTCAGTAGCTATGGCATGTCTTGGGTTCGCCAGACTCCAGACAAGAGGCTGG |
| | AGTGGGTCGCAACCATTAGTAGTGATGGTAGTCACACCTACTATCCAGACAGTGTGAAGGGGCGATTC |
| | ACCATCTCCAGAGACATTGCCAAGAACACCCTGTACCTGCAAATGAGCAGTCTGAAGTCTGAGGACAC |
| | AGCCATGTATTACTGTGTAAGACAGGGGAGGTACGTCCCCTCCTTCGATGTCTGGGGCGCAGGGACCA |
| | CGGTCACCGTCTCCTCA [SEQ ID NO: 16] |
| DNA for | GAAGTTCAACTGGTTGAGTCTGGAGGTGATTTGGTTAAGCCTGGTGGATCTTTGAAGCTTTCATGTGC |
| Full V_{H} | TGCTAGCGGCTTTACGTTTTCCAGCTATGGAATGTCATGGGTGAGACAGACCCCGGACAAGAGGCTCG |
| | AGTGGGTTGCGACTATATCAAGCGATGGAAGTCACACCTACTATCCTGATTCCGTTAAAGGGAGATTC |
| | ACTATCTCCAGGGACATCGCCAAGAACACCCTGTATCTGCAGATGTCCTCTCTCAAATCAGAGGATAC |
| | TGCGATGTATTATTGCGTGCGCCAAGGGAGATATGTACCATCTTTCGACGTATGGGGCGCAGGCACCA |
| | CCGTCACCGTAAGCAGC [SEQ ID NO: 118] |
| DNA for | GATATTGTGCTAACTCAGTCTCCCGCCACCCTGTCTGTGACTCCAGGAGATAGCGTCAGTCTTTCCTG |
| Full V_{L} | CAGGGCCAGCCAAAGTATTAGAAACAACCTACACTGGTATCAACAAAAATCACATGAGTCTCCAAGGC |
| | TTCTCATCAAGGATGCTTCCCAGTCCATTTCTGGGACCCCCTCCAGGTTCAGTGGCAGTGGCTCAGGG |
| | ACAGATTTCACTCTCAGTATCAACAGTGTGGAGACTGAAGATTTTGGAATGTATTTCTGTCACCAGAC |
| | TAACAATTGGCCATTCACGTTCGGCTCGGGGACAAAGTTGGAAATAAAA [SEQ ID NO: 17] |
| DNA for | GATATTGTCCTGACGCAAAGTCCGGCGACCCTGTCAGTTACGCCCGGAGACAGCGTTTCTTTGTCATG |
| Full V_{L} | TAGAGCTAGTCAGTCAATTCGAAATAATCTTCATTGGTATCAACAAAAGAGCCACGAGTCTCCGCGCT |
| | TGTTGATCAAAGACGCTTCCCAGTCAATAAGTGGTACCCCGAGTCGCTTCTCTGGTTCCGGAAGTGGC |
| | ACAGACTTTACGCTTTCTATCAACTCCGTAGAGACTGAGGATTTCGGCATGTATTTTTGCCATCAAAC |
| | GAATAATTGGCCTTTCACGTTTGGATCTGGTACTAAACTGGAAATAAAG [SEQ ID NO: 119] |

In certain embodiments, the anti-CD40 scFv comprises a V_{H} comprising the amino acid sequence set forth in SEQ ID NO: 24, as shown in Table 2. An exemplary nucleotide sequence encoding the amino acid sequence of SEQ ID NO: 24 is set forth in SEQ ID NO: 26 or SEQ ID NO: 120. In certain embodiments, the anti-CD40 scFv comprises a V_{L} comprising the amino acid sequence set forth in SEQ ID NO: 25. An exemplary nucleotide sequence encoding the amino acid sequence of SEQ ID NO: 25 is set forth in SEQ ID NO: 27 or SEQ ID NO: 121. In certain embodiments, the anti-CD40 scFv comprises a V_{H} comprising the amino acid sequence set forth in SEQ ID NO: 24 and a V_{L} comprising the amino acid sequence set forth in SEQ ID NO: 25. SEQ ID NO: 24-27, 120, and 121 are provided in Table 2. In certain embodiments, the scFv is designated as “26-I22”.

In certain embodiments, the anti-CD40 scFv comprises a V_{H} Comprising a CDR1 comprising the amino acid sequence set forth in SEQ ID NO: 18 or a conservative modification thereof, a CDR2 comprising the amino acid sequence set forth in SEQ ID NO: 19 or a conservative modification thereof, and a CDR3 comprising the amino acid sequence set forth in SEQ ID NO: 20 or a conservative modification thereof. SEQ ID NOs: 18-20 are provided in Table 2.

In certain embodiments, the anti-CD40 scFv comprises a V_{L} comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification thereof. SEQ ID NOs: 21-23 are provided in Table 2.

In certain embodiments, the anti-CD40 scFv comprises a V_{H} Comprising a CDR1 comprising the amino acid sequence set forth in SEQ ID NO: 18 or a conservative modification thereof, a CDR2 comprising the amino acid sequence set forth in SEQ ID NO: 19 or a conservative modification thereof, and a CDR3 comprising the amino acid sequence set forth in SEQ ID NO: 20 or a conservative modification thereof; and a V_{L} comprising a CDR1 comprising the amino acid sequence set forth in SEQ ID NO: 21 or a conservative modification thereof, a CDR2 comprising the amino acid sequence set forth in SEQ ID NO: 22 or a conservative modification thereof, and a CDR3 comprising the amino acid sequence set forth in SEQ ID NO: 23 or a conservative modification.

In certain embodiments, the anti-CD40 scFv comprises a V_{H} Comprising a CDR1 comprising the amino acid sequence set forth in SEQ ID NO: 18, a CDR2 comprising the amino acid sequence set forth in SEQ ID NO: 19, and a CDR3 comprising the amino acid sequence set forth in SEQ ID NO: 20; and a V_{L} comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23.

In certain embodiments, the anti-CD40 scFv comprises a V_{H} comprising the amino acid sequence set forth in SEQ ID NO: 24, and a V_{L} comprising the amino acid sequence set forth in SEQ ID NO: 25. In certain embodiments, the V_{H} and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 2
(26-I22)

| CDRs | 1 | 2 | 3 |
|---|---|---|---|
| VI | GYTFINYW [SEQ ID | IYPGSSST [SEQ ID | ARSVGYWYFDV [SEQ ID NO: |
| | NO: 18] | NO: 19] | 20] |
| VI | QSLFFSGNQKNF [SEQ | WAS [SEQ ID NO: 22] | QQYYSYPFT [SEQ ID NO: 23] |
| | ID NO: 21] | | |

| | |
|---|---|
| Full _{VH} | QVQLQQPGAELVKPGASVKLSCKASGYTFINYWINWMRQRPGQGLEWIGNIYPGSSSTTYNEKFKS |
| | KAALTVDTSSSTAYMQLSSLTSDDSAVYFCARSVGYWYFDVWGAGTTVTVSS [SEQ ID NO: |
| | 24] |
| Full V_{L} | DIVMSQSPSSLAVSVGEKVTLSCKSSQSLFFSGNQKNFLAWYQQKPGQSPNLLIFWASTRESGVPD |
| | RFTGSGSGTDFTLTISSVKAEDLAVYYCQQYYSYPFTFGSGTKLEIK [SEQ ID NO: 25] |
| DNA for | CAGGTCCAACTGCAGCAGCCTGGTGCTGAGCTTGTGAAGCCTGGGGCCTCAGTGAAGCTGTCCTGC |
| Full V_{H} | AAGGCTTCCGGCTACACTTTCATTAATTACTGGATAAATTGGATGAGACAGAGGCCTGGACAAGGC |
| | CTTGAATGGATTGGAAATATTTATCCTGGTAGTAGTAGTACTACATACAATGAGAAGTTCAAGAGC |
| | AAGGCCGCACTGACTGTAGACACATCCTCCAGCACAGCCTACATGCAGCTCAGCAGCCTGACATCT |
| | GACGACTCTGCGGTCTATTTTTGTGCAAGATCTGTTGGCTACTGGTACTTCGATGTCTGGGGCGCA |
| | GGGACCACGGTCACCGTCTCCTCA [SEQ ID NO: 26] |
| DNA for | CAGGTACAGCTGCAGCAACCAGGTGCCGAATTGGTCAAACCCGGTGCAAGCGTAAAACTTAGTTGT |
| Full V_{H} | AAAGCGAGCGGCTACACATTCATCAATTATTGGATCAACTGGATGCGACAGCGCCCAGGTCAGGGA |
| | CTTGAATGGATAGGTAATATTTATCCGGGTTCCTCAAGTACAACGTACAACGAGAAATTTAAGTCA |
| | AAAGCTGCATTGACCGTGGATACGTCCAGCTCCACTGCGTACATGCAGCTCAGTTCACTTACGTCT |
| | GATGATTCCGCCGTATATTTCTGCGCTCGGAGCGTGGGTTACTGGTACTTTGACGTATGGGGGGCT |
| | GGAACCACAGTTACGGTTAGTAGC [SEQ ID NO: 120] |
| DNA for | GACATTGTGATGTCACAGTCTCCATCCTCCCTAGCTGTGTCAGTTGGAGAGAAGGTTACTCTGAGC |
| Full V_{L} | TGCAAGTCCAGTCAGAGCCTTTTTTTTAGTGGCAATCAAAAGAACTTCTTGGCCTGGTACCAGCAG |
| | AAACCAGGGCAGTCTCCTAACCTGCTGATTTTCTGGGCATCCACTAGGGAATCTGGGGTCCCTGAT |
| | CGCTTCACAGGCAGTGGATCTGGGACAGATTTCACTCTCACCATCAGCAGTGTGAAGGCTGAAGAC |
| | CTGGCAGTTTATTACTGTCAGCAATATTATAGCTATCCATTCACGTTCGGCTCGGGGACAAAGCTG |
| | GAAATAAAA [SEQ ID NO: 27] |
| DNA for | GACATAGTTATGTCACAGTCTCCATCCTCACTTGCCGTCTCAGTCGGCGAAAAGGTAACACTGAGT |
| Full V_{L} | TGCAAGTCTAGCCAGAGTCTTTTTTTTTCAGGTAACCAAAAAAACTTTCTTGCATGGTACCAGCAG |
| | AAGCCAGGACAGTCACCGAACCTCCTGATATTTTGGGCAAGCACGCGCGAGTCAGGTGTACCGGAC |
| | AGGTTCACCGGTAGTGGCAGCGGAACTGATTTCACACTCACGATAAGTTCCGTGAAGGCTGAGGAT |
| | TTGGCTGTGTACTATTGCCAACAATACTATAGCTATCCTTTCACGTTTGGTTCTGGCACGAAGCTG |
| | GAAATCAAG [SEQ ID NO: 121] |

In certain embodiments, the anti-CD40 scFv comprises a V_{H} comprising the amino acid sequence set forth in SEQ ID NO: 34, as shown in Table 3. An exemplary nucleotide sequence encoding the amino acid sequence of SEQ ID NO: 34 is set forth in SEQ ID NO: 36. In certain embodiments, the anti-CD40 scFv comprises a V_{L} comprising the amino acid sequence set forth in SEQ ID NO: 35. An exemplary nucleotide sequence encoding the amino acid sequence of SEQ ID NO: 35 is set forth in SEQ ID NO: 37. SEQ ID NO: 34-37 are provided in Table 3. In certain embodiments, the scFv is designated as “39-K17”.

In certain embodiments, the anti-CD40 scFv comprises a V_{H} comprising the amino acid sequence set forth in SEQ ID NO: 34 and a V_{L} comprising the amino acid sequence set forth in SEQ ID NO: 35.

In certain embodiments, the anti-CD40 scFv comprises a V_{H} Comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof. SEQ ID NOs: 28-30 are provided in Table 3.

In certain embodiments, the anti-CD40 scFv comprises a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof. SEQ ID NOs: 31-33 are provided in Table 3.

In certain embodiments, the anti-CD40 scFv comprises a V_{H} Comprising a CDR1 comprising the amino acid sequence set forth in SEQ ID NO: 28 or a conservative modification thereof, a CDR2 comprising the amino acid sequence set forth in SEQ ID NO: 29 or a conservative modification thereof, and a CDR3 comprising the amino acid sequence set forth in SEQ ID NO: 30 or a conservative modification thereof; and a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof.

In certain embodiments, the anti-CD40 scFv comprises a V_{H} Comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a V_{L} comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33.

In certain embodiments, the anti-CD40 scFv comprises a V_{H} comprising the amino acid sequence set forth in SEQ ID NO: 34, and a V_{L} comprising the amino acid sequence set forth in SEQ ID NO: 35. In certain embodiments, the V_{H} and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 3
(39-K17)

| CDRs | 1 | 2 | 3 |
|---|---|---|---|
| V_{H} | GYIFTNYW [SEQ ID | IHPSDSES [SEQ ID | ARSPLTKVVTTDF [SEQ ID NO: |
| | NO: 28] | NO: 29] | 30] |
| V_{L} | SSVNY [SEQ ID NO: | DTS [SEQ ID NO: 32] | QQWSSNPLT [SEQ ID NO: 33] |
| | 31] | | |

| | |
|---|---|
| Full V_{H} | QVQLQQPGAELVKPGASVKLSCTASGYIFTNYWMNWVKQRPGRGLEWIGRIHPSDSESLYNQKFKSKA |
| | ILTVDKSSSTAYIQLSSLTSEDSAVYYCARSPLTKVVTTDFWGQGTTLTVSS [SEQ ID NO: 34] |
| Full V_{L} | EIVLTQSPAIMPASPGEKVTMTCSASSSVNYMHWYQQKSGTSPKRWIYDTSKLASGVPVRFSGSGSGT |
| | SYSLTISSMEAEDAATYYCQQWSSNPLTFGGGTKLEIK [SEQ ID NO: 35] |
| DNA for | CAGGTCCAATTGCAGCAGCCTGGGGCTGAACTGGTGAAGCCTGGGGCTTCAGTGAAGCTGTCCTGCAC |
| Full V_{H} | GGCTTCTGGCTACATTTTCACCAACTACTGGATGAACTGGGTGAAGCAGAGGCCTGGACGAGGCCTCG |
| | AGTGGATTGGAAGGATTCATCCTTCTGATAGTGAAAGTCTCTACAATCAAAAGTTCAAGAGCAAGGCC |
| | ATACTGACTGTAGACAAATCCTCCAGCACAGCCTACATCCAACTCAGCAGCCTGACATCTGAGGACTC |
| | TGCGGTCTATTACTGTGCAAGATCCCCTCTTACTAAGGTTGTAACTACGGACTTCTGGGGCCAAGGCA |
| | CCACTCTCACAGTCTCCTCA [SEQ ID NO: 36] |
| DNA for | GAAATTGTTCTCACCCAGTCTCCAGCAATCATGCCTGCATCTCCAGGGGAGAAGGTCACCATGACCTG |
| Full V_{L} | CAGTGCCAGCTCAAGTGTAAATTACATGCACTGGTACCAGCAGAAGTCAGGCACCTCCCCCAAAAGAT |
| | GGATTTATGACACATCCAAACTGGCTTCTGGAGTCCCTGTTCGCTTCAGTGGCAGTGGGTCTGGGACC |
| | TCTTACTCTCTCACAATCAGCAGCATGGAGGCTGAAGATGCTGCCACTTATTACTGCCAGCAGTGGAG |
| | TAGTAACCCACTCACGTTCGGAGGGGGGACCAAGCTGGAAATAAAA [SEQ ID NO: 37] |

In certain embodiments, the anti-CD40 scFv comprises a V_{H} comprising the amino acid sequence set forth in SEQ ID NO: 41, as shown in Table 4. An exemplary nucleotide sequence encoding the amino acid sequence of SEQ ID NO: 41 is set forth in SEQ ID NO: 42. In certain embodiments, the anti-CD40 scFv comprises a V_{L} comprising the amino acid sequence set forth in SEQ ID NO: 35. An exemplary nucleotide sequence encoding the amino acid sequence of SEQ ID NO: 35 is set forth in SEQ ID NO: 37. SEQ ID NO: 35, 37, 41, and 42 are provided in Table 4. In certain embodiments, the scFv is designated as “41-J9”

In certain embodiments, the anti-CD40 scFv comprises a V_{H} comprising the amino acid sequence set forth in SEQ ID NO: 41 and a V_{L} comprising the amino acid sequence set forth in SEQ ID NO: 35. SEQ ID NOs: 41 and 35 are provided in Table 4.

In certain embodiments, the anti-CD40 scFv comprises a V_{H} Comprising a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof. SEQ ID NOs: 38-40 are provided in Table 4.

In certain embodiments, the anti-CD40 scFv comprises a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof. SEQ ID NOs: 31-33 are provided in Table 4.

In certain embodiments, the anti-CD40 scFv comprises a V_{H} Comprising a CDR1 comprising the amino acid sequence set forth in SEQ ID NO: 38 or a conservative modification thereof, a CDR2 comprising the amino acid sequence set forth in SEQ ID NO: 39 or a conservative modification thereof, and a CDR3 comprising the amino acid sequence set forth in SEQ ID NO: 40 or a conservative modification thereof; and a V_{L} comprising a CDR1 comprising the amino acid sequence set forth in SEQ ID NO: 31 or a conservative modification thereof, a CDR2 comprising the amino acid sequence set forth in SEQ ID NO: 32 or a conservative modification thereof, and a CDR3 comprising the amino acid sequence set forth in SEQ ID NO: 33 or a conservative modification thereof.

In certain embodiments, the anti-CD40 scFv comprises a V_{H} Comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40; and a V_{L} comprising a CDR1 comprising the amino acid sequence set forth in SEQ ID NO: 31, a comprising a CDR2 comprising the amino acid sequence set forth in SEQ ID NO: 32, and a CDR3 comprising the amino acid sequence set forth in SEQ ID NO: 33.

In certain embodiments, the anti-CD40 scFv comprises a V_{H} comprising the amino acid sequence set forth in SEQ ID NO: 41, and a V_{L} comprising the amino acid sequence set forth in SEQ ID NO: 35. In certain embodiments, the V_{H} and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 4
(41-J9)

| CDRs | 1 | 2 | 3 |
|---|---|---|---|
| V_{H} | GYSITSDYS [SEQ ID | IYYSGST [SEQ ID NO: | ARFYYGWSYFDY [SEQ ID NO: |
| | NO: 38] | 39] | 40] |
| V_{L} | SSVNY [SEQ ID NO: | DTS [SEQ ID NO: 32] | QQWSSNPLT [SEQ ID NO: 33] |
| | 31] | | |

| | |
|---|---|
| Full V_{H} | DVQLQESGPDLVKPSQSLSLTCTVTGYSITSDYSWHWIRQFPGNKLEWMGYIYYSGSTNYNPSLKSR |
| | ISITRDTSKNQFFLQLNSVTSEDTATYYCARFYYGWSYFDYWGQGTSLTVSS [SEQ ID NO: 41] |
| Full V_{L} | EIVLTQSPAIMPASPGEKVTMTCSASSSVNYMHWYQQKSGTSPKRWIYDTSKLASGVPVRFSGSGSG |
| | TSYSLTISSMEAEDAATYYCQQWSSNPLTFGGGTKLEIK [SEQ ID NO: 35] |
| DNA for | GATGTGCAGCTTCAGGAGTCAGGACCTGACCTGGTGAAACCTTCTCAGTCACTTTCACTCACCTGCA |
| Full V_{H} | CTGTCACTGGCTACTCCATCACCAGTGATTATAGCTGGCACTGGATCCGGCAGTTTCCAGGAAACAA |
| | ACTGGAATGGATGGGCTACATATATTACAGTGGTAGCACTAACTACAACCCATCTCTCAAAAGTCGA |
| | ATCTCTATCACTCGAGACACATCCAAGAACCAGTTCTTCCTGCAACTGAATTCTGTGACTTCTGAGG |
| | ACACAGCCACATATTACTGTGCAAGATTTTACTACGGTTGGAGTTACTTTGACTACTGGGGCCAAGG |
| | CACCAGTCTCACAGTCTCCTCA [SEQ ID NO: 42] |
| DNA for | GAAATTGTTCTCACCCAGTCTCCAGCAATCATGCCTGCATCTCCAGGGGAGAAGGTCACCATGACCT |
| Full V_{L} | GCAGTGCCAGCTCAAGTGTAAATTACATGCACTGGTACCAGCAGAAGTCAGGCACCTCCCCCAAAAG |
| | ATGGATTTATGACACATCCAAACTGGCTTCTGGAGTCCCTGTTCGCTTCAGTGGCAGTGGGTCTGGG |
| | ACCTCTTACTCTCTCACAATCAGCAGCATGGAGGCTGAAGATGCTGCCACTTATTACTGCCAGCAGT |
| | GGAGTAGTAACCCACTCACGTTCGGAGGGGGGACCAAGCTGGAAATAAAA [SEQ ID NO: 37] |

In certain embodiments, the anti-CD40 scFv comprises a V_{H} comprising the amino acid sequence set forth in SEQ ID NO: 49, as shown in Table 5. An exemplary nucleotide sequence encoding the amino acid sequence of SEQ ID NO: 49 is set forth in SEQ ID NO: 51. In certain embodiments, the anti-CD40 scFv comprises a V_{L} comprising the amino acid sequence set forth in SEQ ID NO: 50. An exemplary nucleotide sequence encoding the amino acid sequence of SEQ ID NO: 50 is set forth in SEQ ID NO: 52. SEQ ID NO: 49-52 are provided in Table 5. In certain embodiments, the scFv is designated as “41-E17”.

In certain embodiments, the anti-CD40 scFv comprises a V_{H} comprising the amino acid sequence set forth in SEQ ID NO: 49 and a V_{L} comprising the amino acid sequence set forth in SEQ ID NO: 50.

In certain embodiments, the anti-CD40 scFv comprises a V_{H} Comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof. SEQ ID NOs: 43-45 are provided in Table 5.

In certain embodiments, the anti-CD40 scFv comprises a V_{L} comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof. SEQ ID NOs: 46-48 are provided in Table 5.

In certain embodiments, the anti-CD40 scFv comprises a V_{H} Comprising a CDR1 comprising the amino acid sequence set forth in SEQ ID NO: 43 or a conservative modification thereof, a CDR2 comprising the amino acid sequence set forth in SEQ ID NO: 44 or a conservative modification thereof, and a CDR3 comprising the amino acid sequence set forth in SEQ ID NO: 45 or a conservative modification thereof; and a V_{L} comprising a CDR1 comprising the amino acid sequence set forth in SEQ ID NO: 46 or a conservative modification thereof, a CDR2 comprising the amino acid sequence set forth in SEQ ID NO: 47 or a conservative modification thereof, and a CDR3 comprising the amino acid sequence set forth in SEQ ID NO: 48 or a conservative modification thereof.

In certain embodiments, the anti-CD40 scFv comprises a V_{H} Comprising a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and a V_{L} comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48.

In certain embodiments, the anti-CD40 scFv comprises a V_{H} comprising the amino acid sequence set forth in SEQ ID NO: 49, and a V_{L} comprising the amino acid sequence set forth in SEQ ID NO: 50. In certain embodiments, the V_{H}- and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 5
(41-E17)

| CDRs | 1 | 2 | 3 |
|---|---|---|---|
| V_{H} | GFTFSTFG [SEQ ID | ISGGSSTI [SEQ ID | VRPYGSVHYAMDY |
| | NO: 43] | NO: 44] | [SEQ ID NO: 45] |
| V_{L} | QDINNY [SEQ ID NO: | YTS [SEQ ID NO: 47] | QQGNTLPWT [SEQ ID NO: 48] |
| | 46] | | |

| | |
|---|---|
| Full V_{H} | DVQLVESGGGLVQPGGSRKLSCAASGFTFSTFGMHWVRQAPEKGLEWVAYISGGSSTIYYADTVKGRFT |
| | ISRDNPKNTLFLQMTSLRSEDTAMYYCVRPYGSVHYAMDYWGQGTSVTVSS [SEQ ID NO: 49] |
| Full V_{L} | DIQMTQTTSSLSASLGDRVTISCRASQDINNYLNWYQQKPDGTVKLLIYYTSRFHSGVPSRFSGSGSGT |
| | DYSLTISNLEQEDIATYFCQQGNTLPWTFGGGTKLDIK [SEQ ID NO: 50] |
| DNA for | GATGTGCAGCTGGTGGAGTCTGGGGGAGGCTTAGTGCAGCCTGGAGGGTCCCGGAAACTCTCCTGTGCA |
| Full V_{H} | GCCTCTGGATTCACTTTCAGTACCTTTGGAATGCACTGGGTTCGTCAGGCTCCAGAGAAGGGGCTGGAG |
| | TGGGTCGCATACATTAGTGGTGGCAGTAGTACCATCTACTATGCAGACACAGTGAAGGGCCGATTCACC |
| | ATCTCCAGAGACAATCCCAAGAACACCCTGTTCCTGCAAATGACCAGTCTAAGGTCTGAGGACACGGCC |
| | ATGTATTACTGTGTAAGACCCTACGGTAGTGTTCACTATGCTATGGACTACTGGGGTCAAGGAACCTCA |
| | GTCACCGTCTCCTCA [SEQ ID NO: 51] |
| DNA for | GATATCCAGATGACACAGACTACATCCTCCCTGTCTGCCTCTCTGGGAGACAGAGTCACCATCAGTTGC |
| Full V_{L} | AGGGCAAGTCAGGACATTAACAATTATTTAAACTGGTATCAGCAGAAACCAGATGGAACTGTTAAACTC |
| | CTGATCTACTACACATCAAGATTTCACTCAGGAGTCCCATCAAGGTTCAGTGGCAGTGGGTCTGGAACA |
| | GATTATTCTCTCACCATTAGCAACCTGGAGCAAGAAGATATTGCCACTTACTTTTGCCAACAGGGTAAT |
| | ACGCTTCCGTGGACGTTCGGTGGAGGCACCAAGCTGGACATCAAA [SEQ ID NO: 52] |

In certain embodiments, the anti-CD40 scFv comprises a V_{H} comprising the amino acid sequence set forth in SEQ ID NO: 59, as shown in Table 6. An exemplary nucleotide sequence encoding the amino acid sequence of SEQ ID NO: 59 is set forth in SEQ ID NO: 61. In certain embodiments, the anti-CD40 scFv comprises a V_{L} comprising the amino acid sequence set forth in SEQ ID NO: 60. An exemplary nucleotide sequence encoding the amino acid sequence of SEQ ID NO: 60 is set forth in SEQ ID NO: 62. SEQ ID NO: 59-62 are provided in Table 6. In certain embodiments, the scFv is designated as “30-P15”.

In certain embodiments, the anti-CD40 scFv comprises a V_{H} comprising the amino acid sequence set forth in SEQ ID NO: 59 and a V_{L} comprising the amino acid sequence set forth in SEQ ID NO: 60.

In certain embodiments, the anti-CD40 scFv comprises a V_{H} Comprising a CDR1 comprising the amino acid sequence set forth in SEQ ID NO: 53 or a conservative modification thereof, a CDR2 comprising the amino acid sequence set forth in SEQ ID NO: 54 or a conservative modification thereof, and a CDR3 comprising the amino acid sequence set forth in SEQ ID NO: 55 or a conservative modification thereof. SEQ ID NOs: 53-55 are provided in Table 6.

In certain embodiments, the anti-CD40 scFv comprises a V_{L} comprising a CDR1 comprising the amino acid sequence set forth in SEQ ID NO: 56 or a conservative modification thereof, a CDR2 comprising the amino acid sequence set forth in SEQ ID NO: 57 or a conservative modification thereof, and a CDR3 comprising the amino acid sequence set forth in SEQ ID NO: 58 or a conservative modification thereof. SEQ ID NOs: 56-58 are provided in Table 6.

In certain embodiments, the anti-CD40 scFv comprises a V_{H} Comprising a CDR1 comprising the amino acid sequence set forth in SEQ ID NO: 53 or a conservative modification thereof, a CDR2 comprising the amino acid sequence set forth in SEQ ID NO: 54 or a conservative modification thereof, and a CDR3 comprising the amino acid sequence set forth in SEQ ID NO: 55 or a conservative modification thereof; and a V_{L} comprising a CDR1 comprising the amino acid sequence set forth in SEQ ID NO: 56 or a conservative modification thereof, a CDR2 comprising the amino acid sequence set forth in SEQ ID NO: 57 or a conservative modification thereof, and a CDR3 comprising the amino acid sequence set forth in SEQ ID NO: 58 or a conservative modification thereof.

In certain embodiments, the anti-CD40 scFv comprises a V_{H} Comprising a CDR1 comprising the amino acid sequence set forth in SEQ ID NO: 53, a CDR2 comprising the amino acid sequence set forth in SEQ ID NO: 54, and a CDR3 comprising the amino acid sequence set forth in SEQ ID NO: 55; and a V_{L} comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58.

In certain embodiments, the anti-CD40 scFv comprises a V_{H} comprising the amino acid sequence set forth in SEQ ID NO: 59, and a V_{L} comprising the amino acid sequence set forth in SEQ ID NO: 60. In certain embodiments, the V_{H} and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 6
(30-P15)

| CDRs | 1 | 2 | 3 |
|---|---|---|---|
| V_{H} | GFTFSNYA [SEQ ID | IGTSGSYT [SEQ ID | ARRGYYNPTHAMDY [SEQ ID NO: |
| | NO: 53] | NO: 54] | 55] |
| V_{L} | TGAVTTSNY [SEQ ID | GTN [SEQ ID NO: 57] | ALWYSNHLV [SEQ ID NO: 58] |
| | NO: 56] | | |

| | |
|---|---|
| Full V_{H} | EVMLVESGGALVKPGGSLKLSCAASGFTFSNYAMSWVRQTPEKRLEWVATIGTSGSYTYYSDSVKGR |
| | FTISRDNARNTLYLQMTSLRSEDTAMYYCARRGYYNPTHAMDYWGQGTSVTVSS [SEQ ID NO: |
| | 59] |
| Full V_{L} | QAVVTQESALTTSPGETVTLTCRSSTGAVTTSNYANWVQEKPDHLFTGLIGGTNNRAPGVPARFSGS |
| | LIGDKAALTITGAQTEDEAIYFCALWYSNHLVFGGGTKLTVL [SEQ ID NO: 60] |
| DNA for | GAGGTGATGCTGGTGGAGTCTGGGGGAGCCTTAGTGAAGCCTGGAGGGTCCCTGAAACTCTCCTGTG |
| Full V_{H} | CAGCCTCTGGATTCACTTTCAGTAACTATGCCATGTCTTGGGTTCGCCAGACTCCAGAGAAGAGGCT |
| | GGAGTGGGTCGCAACCATTGGTACTAGTGGTAGTTACACCTACTATTCAGACAGTGTGAAGGGTCGA |
| | TTCACCATCTCCAGAGACAATGCCAGGAACACCCTGTACCTGCAAATGACCAGTCTGAGGTCTGAGG |
| | ACACGGCCATGTATTACTGTGCAAGACGGGGGTACTACAATCCGACCCATGCTATGGACTACTGGGG |
| | TCAAGGAACCTCAGTCACCGTCTCCTCA [SEQ ID NO: 61] |
| DNA for | CAGGCTGTTGTGACTCAGGAATCTGCACTCACCACATCACCTGGTGAAACAGTCACACTCACTTGTC |
| Full V_{L} | GCTCAAGTACTGGGGCTGTTACAACTAGTAACTATGCCAACTGGGTCCAAGAAAAACCAGATCATTT |
| | ATTCACTGGTCTAATAGGTGGTACCAACAACCGAGCTCCAGGTGTTCCTGCCAGATTCTCAGGCTCC |
| | CTGATTGGAGACAAGGCTGCCCTCACCATCACAGGGGCACAGACTGAGGATGAGGCAATATATTTCT |
| | GTGCTCTATGGTACAGCAACCATTTGGTGTTCGGTGGAGGAACCAAACTGACTGTCCTAG [SEQ ID |
| | NO: 62] |

In certain embodiments, the anti-CD40 scFv comprises a V_{H} comprising the amino acid sequence set forth in SEQ ID NO: 68, as shown in Table 7. An exemplary nucleotide sequence encoding the amino acid sequence of SEQ ID NO: 68 is set forth in SEQ ID NO: 70. In certain embodiments, the anti-CD40 scFv comprises a V_{L} comprising the amino acid sequence set forth in SEQ ID NO: 69. An exemplary nucleotide sequence encoding the amino acid sequence of SEQ ID NO: 69 is set forth in SEQ ID NO: 71. SEQ ID NO: 68-71 are provided in Table 7. In certain embodiments, the scFv is designated as “12-O17”.

In certain embodiments, the anti-CD40 scFv comprises a V_{H} comprising the amino acid sequence set forth in SEQ ID NO: 68 and a V_{L} comprising the amino acid sequence set forth in SEQ ID NO: 69.

In certain embodiments, the anti-CD40 scFv comprises a V_{H} Comprising a CDR1 comprising the amino acid sequence set forth in SEQ ID NO: 63 or a conservative modification thereof, a CDR2 comprising the amino acid sequence set forth in SEQ ID NO: 64 or a conservative modification thereof, and a CDR3 comprising the amino acid sequence set forth in SEQ ID NO: 65 or a conservative modification thereof. SEQ ID NOs: 63-65 are provided in Table 7.

In certain embodiments, the anti-CD40 scFv comprises a V_{L} comprising a CDR1 comprising the amino acid sequence set forth in SEQ ID NO: 66 or a conservative modification thereof, a CDR2 comprising the amino acid sequence set forth in SEQ ID NO: 22 or a conservative modification thereof, and a CDR3 comprising the amino acid sequence set forth in SEQ ID NO: 67 or a conservative modification thereof. SEQ ID NOs: 22, 66, and 67 are provided in Table 7.

In certain embodiments, the anti-CD40 scFv comprises a V_{H} Comprising a CDR1 comprising the amino acid sequence set forth in SEQ ID NO: 63 or a conservative modification thereof, a CDR2 comprising the amino acid sequence set forth in SEQ ID NO: 64 or a conservative modification thereof, and a CDR3 comprising the amino acid sequence set forth in SEQ ID NO: 65 or a conservative modification thereof; and a V_{L} comprising a CDR1 comprising the amino acid sequence set forth in SEQ ID NO: 66 or a conservative modification thereof, a CDR2 comprising the amino acid sequence set forth in SEQ ID NO: 22 or a conservative modification thereof, and a CDR3 comprising the amino acid sequence set forth in SEQ ID NO: 67 or a conservative modification thereof.

In certain embodiments, the anti-CD40 scFv comprises a V_{H} Comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65; a V_{L} comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67.

In certain embodiments, the anti-CD40 scFv comprises a V_{H} comprising the amino acid sequence set forth in SEQ ID NO: 68 and a V_{L} comprising the amino acid sequence set forth in SEQ ID NO: 69. In certain embodiments, the V_{H} and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 7
(12-O17)

| CDRs | 1 | 2 | 3 |
|---|---|---|---|
| V_{H} | GYTFSTYW [SEQ ID | ILPGSGST [SEQ ID | ARGGLYYYGGAMDY [SEQ ID NO: |
| | NO: 63] | NO: 64] | 65] |
| V_{L} | QSLLYSGNQKNY [SEQ | WAS [SEQ ID NO: 22] | QQYYSYPYT [SEQ ID NO: 67] |
| | ID NO: 66] | | |

| | |
|---|---|
| Full V_{H} | QVQLQQSGAELMKPGASVKISCKATGYTFSTYWIEWVKQRPGHGLEWIGEILPGSGSTNYNENFKGK |
| | ATFTADTSSNTAYMQLSSLTSEDSAVYFCARGGLYYYGGAMDYWGQGTSVTVSS [SEQ ID NO: |
| | 68] |
| Full V_{L} | DIVMSQSPSSLAVSVGEKVTMSCKSSQSLLYSGNQKNYLAWYQQKPGQSPKLLIYWASIRESGVPDR |
| | FTGSGSGTDFTLTISSVKAEDLAVYYCQQYYSYPYTFGGGTKVEIK [SEQ ID NO: 69] |
| DNA for | CAGGTTCAGCTGCAGCAGTCTGGAGCTGAGCTGATGAAGCCTGGGGCCTCAGTGAAGATATCCTGCA |
| Full V_{H} | AGGCTACTGGCTACACATTCAGTACCTACTGGATAGAGTGGGTAAAGCAGAGGCCTGGACATGGCCT |
| | TGAGTGGATTGGAGAGATTTTACCTGGAAGTGGTAGTACTAACTACAATGAGAACTTCAAGGGCAAG |
| | GCCACATTCACTGCAGATACATCCTCCAACACAGCCTACATGCAACTCAGCAGCCTGACATCTGAGG |
| | ACTCTGCCGTCTATTTCTGTGCTAGAGGGGGACTTTATTACTACGGAGGGGCTATGGACTACTGGGG |
| | TCAAGGAACCTCAGTCACCGTCTCCTCA [SEQ ID NO: 70] |
| DNA for | GACATTGTGATGTCACAGTCTCCATCCTCCCTCGCTGTGTCAGTTGGAGAGAAGGTTACTATGAGCT |
| Full V_{L} | GCAAGTCCAGTCAGAGCCTTTTATATAGTGGCAATCAAAAGAACTACTTGGCCTGGTACCAGCAGAA |
| | ACCAGGGCAGTCTCCTAAACTGCTGATTTACTGGGCATCCATTAGGGAATCTGGGGTCCCTGATCGC |
| | TTCACAGGCAGTGGATCTGGGACAGATTTCACTCTCACCATCAGCAGTGTGAAGGCTGAAGACCTGG |
| | CAGTTTATTACTGTCAGCAATATTATAGCTATCCGTACACGTTCGGAGGGGGGACCAAGGTGGAAAT |
| | AAAA [SEQ ID NO: 71] |

In certain embodiments, the anti-CD40 scFv comprises a V_{H} comprising the amino acid sequence set forth in SEQ ID NO: 78, as shown in Table 8. An exemplary nucleotide sequence encoding the amino acid sequence of SEQ ID NO: 78 is set forth in SEQ ID NO: 80. In certain embodiments, the anti-CD40 scFv comprises a V_{L} comprising the amino acid sequence set forth in SEQ ID NO: 79. An exemplary nucleotide sequence encoding the amino acid sequence of SEQ ID NO: 79 is set forth in SEQ ID NO: 81. SEQ ID NO: 78-81 are provided in Table 8. In certain embodiments, the scFv is designated as “41-I22”.

In certain embodiments, the anti-CD40 scFv comprises a V_{H} comprising the amino acid sequence set forth in SEQ ID NO: 78 and a V_{L} comprising the amino acid sequence set forth in SEQ ID NO: 79.

In certain embodiments, the anti-CD40 scFv comprises a V_{H} Comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof. SEQ ID NOs: 72-74 are provided in Table 8.

In certain embodiments, the anti-CD40 scFv comprises a V_{L} comprising a CDR1 comprising the amino acid sequence set forth in SEQ ID NO: 75 or a conservative modification thereof, a CDR2 comprising the amino acid sequence set forth in SEQ ID NO: 76 or a conservative modification thereof, and a CDR3 comprising the amino acid sequence set forth in SEQ ID NO: 77 or a conservative modification thereof. SEQ ID NOs: 75-77 are provided in Table 8.

In certain embodiments, the anti-CD40 scFv comprises a V_{H} Comprising a CDR1 comprising the amino acid sequence set forth in SEQ ID NO: 72 or a conservative modification thereof, a CDR2 comprising the amino acid sequence set forth in SEQ ID NO: 73 or a conservative modification thereof, and a CDR3 comprising the amino acid sequence set forth in SEQ ID NO: 74 or a conservative modification thereof; and a V_{L} comprising a CDR1 comprising the amino acid sequence set forth in SEQ ID NO: 75 or a conservative modification thereof, a CDR2 comprising the amino acid sequence set forth in SEQ ID NO: 76 or a conservative modification thereof, and a CDR3 comprising the amino acid sequence set forth in SEQ ID NO: 77 or a conservative modification thereof.

In certain embodiments, the anti-CD40 scFv comprises a V_{H} Comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and a V_{L} comprising a CDR1 comprising the amino acid sequence set forth in SEQ ID NO: 75, a CDR2 comprising the amino acid sequence set forth in SEQ ID NO: 76, and a CDR3 comprising the amino acid sequence set forth in SEQ ID NO: 77.

In certain embodiments, the anti-CD40 scFv comprises a V_{H} comprising the amino acid sequence set forth in SEQ ID NO: 78, and a V_{L} comprising the amino acid sequence set forth in SEQ ID NO: 79. In certain embodiments, the V_{H} and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 8
(41-I22)

| CDRs | 1 | 2 | 3 |
|---|---|---|---|
| V_{H} | GFNIKDTY [SEQ ID | IDPANGNT [SEQ ID | ARDYYGTIFPYWYFDV [SEQ ID NO: |
| | NO: 72] | NO: 73] | 74] |
| V_{L} | SSVSY [SEQ ID NO: | ITS [SEQ ID NO: 76] | QQRSSYPLT [SEQ ID NO: 77] |
| | 75] | | |

| | |
|---|---|
| Full V_{H} | EVQLQQSGAVLVKPGASVRLSCTASGFNIKDTYIHWVKQRPEQGLEWIGKIDPANGNTKFDPKFQGKA |
| | TIAADTSSNTAYLQLSSLTSEDTAVYYCARDYYGTIFPYWYFDVWGAGTTVTVSS [SEQ ID NO: |
| | 78] |
| Full V_{L} | QIVLTQSPAIMSASPGEKVTITCSASSSVSYMHWFQQKPGTSPKFWIYITSNLASGVPARFSGSGSGT |
| | SYSLTISRMEAEDAATYYCQQRSSYPLTFGAGTKLELK [SEQ ID NO: 79] |
| DNA for | GAGGTTCAGCTGCAGCAGTCTGGGGCAGTACTTGTGAAGCCAGGGGCCTCAGTCAGGTTGTCCTGTAC |
| Full V_{H} | AGCTTCTGGCTTCAACATTAAAGACACCTATATACACTGGGTGAAGCAGAGGCCTGAACAGGGCCTGG |
| | AGTGGATTGGAAAGATTGATCCTGCGAATGGTAATACTAAATTTGACCCGAAGTTCCAGGGCAAGGCC |
| | ACTATAGCAGCAGACACATCCTCCAACACAGCCTACCTGCAGCTCAGCAGCCTGACATCTGAGGACAC |
| | TGCCGTCTATTACTGTGCTAGAGATTACTACGGTACTATCTTCCCTTACTGGTACTTCGATGTCTGGG |
| | GCGCAGGGACCACGGTCACCGTCTCCTCA [SEQ ID NO: 80] |
| DNA for | CAAATTGTTCTCACCCAGTCTCCAGCAATCATGTCTGCATCTCCAGGGGAGAAGGTCACCATAACCTG |
| Full V_{L} | CAGTGCCAGCTCAAGTGTAAGTTACATGCACTGGTTCCAGCAGAAGCCAGGCACTTCTCCCAAATTCT |
| | GGATTTATATCACATCCAACCTGGCTTCTGGAGTCCCTGCTCGCTTCAGTGGCAGTGGATCTGGGACC |
| | TCTTACTCTCTCACAATCAGCCGAATGGAGGCTGAAGATGCTGCCACTTATTACTGCCAGCAAAGGAG |
| | TAGTTACCCACTCACGTTCGGTGCTGGGACCAAGCTGGAGCTGAAA [SEQ ID NO: 81] |

In certain embodiments, the anti-CD40 scFv comprises a V_{H} comprising the amino acid sequence set forth in SEQ ID NO: 83, as shown in Table 9. An exemplary nucleotide sequence encoding the amino acid sequence of SEQ ID NO: 83 is set forth in SEQ ID NO: 85. In certain embodiments, the anti-CD40 scFv comprises a V_{L} comprising the amino acid sequence set forth in SEQ ID NO: 84. An exemplary nucleotide sequence encoding the amino acid sequence of SEQ ID NO: 84 is set forth in SEQ ID NO: 86. SEQ ID NO: 83-86 are provided in Table 9. In certain embodiments, the scFv is designated as “9-P1”.

In certain embodiments, the anti-CD40 scFv comprises a V_{H} comprising the amino acid sequence set forth in SEQ ID NO: 83 and a V_{L} comprising the amino acid sequence set forth in SEQ ID NO: 84.

In certain embodiments, the anti-CD40 scFv comprises a V_{H} Comprising a CDR1 comprising the amino acid sequence set forth in SEQ ID NO: 63 or a conservative modification thereof, a CDR2 comprising the amino acid sequence set forth in SEQ ID NO: 64 or a conservative modification thereof, and a CDR3 comprising the amino acid sequence set forth in SEQ ID NO: 82 or a conservative modification thereof. SEQ ID NOs: 63, 64, and 82 are provided in Table 9.

In certain embodiments, the anti-CD40 scFv comprises a V_{L} comprising a CDR1 comprising the amino acid sequence set forth in SEQ ID NO: 66 or a conservative modification thereof, a CDR2 comprising the amino acid sequence set forth in SEQ ID NO: 22 or a conservative modification thereof, and a CDR3 comprising the amino acid sequence set forth in SEQ ID NO: 67 or a conservative modification thereof. SEQ ID NOs: 22, 66, and 67 are provided in Table 9.

In certain embodiments, the anti-CD40 scFv comprises a V_{H} Comprising a CDR1 comprising the amino acid sequence set forth in SEQ ID NO: 63 or a conservative modification thereof, a CDR2 comprising the amino acid sequence set forth in SEQ ID NO: 64 or a conservative modification thereof, and a CDR3 comprising the amino acid sequence set forth in SEQ ID NO: 82 or a conservative modification thereof; and a V_{L} comprising a CDR1 comprising the amino acid sequence set forth in SEQ ID NO: 66 or a conservative modification thereof, a CDR2 comprising the amino acid sequence set forth in SEQ ID NO: 22 or a conservative modification thereof, and a CDR3 comprising the amino acid sequence set forth in SEQ ID NO: 67 or a conservative modification thereof.

In certain embodiments, the anti-CD40 scFv comprises a V_{H} Comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 82; and a V_{L} comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67.

In certain embodiments, the anti-CD40 scFv comprises a V_{H} comprising the amino acid sequence set forth in SEQ ID NO: 83, and a V_{L} comprising the amino acid sequence set forth in SEQ ID NO: 84. In certain embodiments, the V_{H} and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 9
(9-P1)

| CDRs | 1 | 2 | 3 |
|---|---|---|---|
| V_{H} | GYTFSTYW [SEQ ID | ILPGSGST [SEQ ID | ARSYYTYDQGSWLAY [SEQ ID NO: |
| | NO: 63] | NO: 64] | 82] |
| V_{L} | QSLLYSGNQKNY [SEQ | WAS [SEQ ID NO: 22] | QQYYSYPYT [SEQ ID NO: 67] |
| | ID NO: 66] | | |

| | |
|---|---|
| Full V_{H} | QVQLQQSGAELMKPGASVKISCKATGYTESTYWIEWVKQRPGHGLEWIGEILPGSGSTNYNEKFRGK |
| | ATFTADTSSNTAYMQLSSLTSEDSAVYYCARSYYTYDQGSWLAYWGQGTLVTVSA [SEQ ID NO: |
| | 83] |
| Full V_{L} | DIVMSQSPSSLAVSVGEKVTMTCKSSQSLLYSGNQKNYLAWYQQKPGQSPKLLINWASTRESGVPDR |
| | LTGSGSGTDFSLTISSVKAEDLAVYYCQQYYSYPYTFGGGTKLEIK [SEQ ID NO: 84] |
| DNA for | CAGGTTCAGCTGCAGCAGTCTGGAGCTGAGCTGATGAAGCCTGGGGCCTCAGTGAAGATATCCTGCA |
| Full V_{H} | AGGCTACTGGCTACACATTCAGTACCTACTGGATAGAGTGGGTAAAGCAGAGGCCTGGACATGGCCT |
| | TGAGTGGATTGGAGAGATTTTACCTGGAAGTGGTAGTACTAACTACAATGAGAAGTTCAGGGGCAAG |
| | GCCACATTCACTGCAGATACATCCTCCAACACAGCCTACATGCAACTCAGCAGCCTGACATCTGAGG |
| | ACTCTGCCGTCTATTACTGTGCAAGATCCTACTATACGTACGACCAGGGGTCCTGGCTTGCTTACTG |
| | GGGCCAAGGGACTCTGGTCACTGTCTCTGCA [SEQ ID NO: 85] |
| DNA for | GACATTGTGATGTCACAGTCTCCATCCTCCCTAGCTGTGTCAGTTGGAGAGAAGGTTACTATGACCT |
| Full V_{L} | GCAAGTCCAGTCAGAGCCTTTTATATAGTGGCAATCAAAAGAACTACTTGGCCTGGTACCAGCAGAA |
| | ACCAGGGCAGTCTCCTAAACTGCTGATTAACTGGGCATCTACTAGGGAATCTGGGGTCCCTGATCGC |
| | TTAACAGGCAGTGGATCTGGGACAGATTTCAGTCTCACCATCAGCAGTGTGAAGGCTGAAGACCTGG |
| | CAGTTTATTACTGTCAGCAATATTATAGCTATCCGTACACGTTCGGAGGGGGGACCAAGCTGGAAAT |
| | AAAA [SEQ ID NO: 86] |

In certain embodiments, the anti-CD40 scFv comprises a V_{H} comprising the amino acid sequence set forth in SEQ ID NO: 93, as shown in Table 10. An exemplary nucleotide sequence encoding the amino acid sequence of SEQ ID NO: 93 is set forth in SEQ ID NO: 95. In certain embodiments, the anti-CD40 scFv comprises a V_{L} comprising the amino acid sequence set forth in SEQ ID NO: 94. An exemplary nucleotide sequence encoding the amino acid sequence of SEQ ID NO: 94 is set forth in SEQ ID NO: 96. SEQ ID NO: 93-96 are provided in Table 10. In certain embodiments, the scFv is designated as “10-B8”.

In certain embodiments, the anti-CD40 scFv comprises a V_{H} comprising the amino acid sequence set forth in SEQ ID NO: 93 and a V_{L} comprising the amino acid sequence set forth in SEQ ID NO: 94.

In certain embodiments, the anti-CD40 scFv comprises a V_{H} Comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof. SEQ ID NOs: 87-89 are provided in Table 10.

In certain embodiments, the anti-CD40 scFv comprises a V_{L} comprising a CDR1 comprising the amino acid sequence set forth in SEQ ID NO: 90 or a conservative modification thereof, a CDR2 comprising the amino acid sequence set forth in SEQ ID NO: 91 or a conservative modification thereof, and a CDR3 comprising the amino acid sequence set forth in SEQ ID NO: 92 or a conservative modification thereof. SEQ ID NOs: 90-92 are provided in Table 10.

In certain embodiments, the anti-CD40 scFv comprises a V_{H} Comprising a CDR1 comprising the amino acid sequence set forth in SEQ ID NO: 87 or a conservative modification thereof, a CDR2 comprising the amino acid sequence set forth in SEQ ID NO: 88 or a conservative modification thereof, and a CDR3 comprising the amino acid sequence set forth in SEQ ID NO: 89 or a conservative modification thereof, a V_{L} comprising a CDR1 comprising the amino acid sequence set forth in SEQ ID NO: 90 or a conservative modification thereof, a CDR2 comprising the amino acid sequence set forth in SEQ ID NO: 91 or a conservative modification thereof, and a CDR3 comprising the amino acid sequence set forth in SEQ ID NO: 92 or a conservative modification thereof.

In certain embodiments, the anti-CD40 scFv comprises a V_{H} Comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; a V_{L} comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92.

In certain embodiments, the anti-CD40 scFv comprises a V_{H} comprising the amino acid sequence set forth in SEQ ID NO: 93, and a V_{L} comprising the amino acid sequence set forth in SEQ ID NO: 94. In certain embodiments, the V_{H} and V_{L} are linked via a linker. In certain embodiments, the linker comprises the amino acid sequence set forth in SEQ ID NO: 2.

In certain embodiments, a heavy chain variable region (V_{H}) is positioned at the N-terminus. In certain embodiments, the variable regions are positioned from the N- to the C-terminus: V_{H}-V_{L}. In certain embodiments, a light chain variable region (V_{L}) is positioned at the N-terminus. In certain embodiments, the variable regions are positioned from the N- to the C-terminus: V_{L}-V_{H}.

TABLE 10
(10-B8)

| CDRs | 1 | 2 | 3 |
|---|---|---|---|
| V_{H} | GFTFATSW [SEQ ID | IHPNSGNT [SEQ ID | ARGADYAMDY [SEQ ID NO: 89] |
| | NO: 87] | NO: 88] | |
| V_{L} | QSIVHSNGNTY [SEQ ID | IVS [SEQ ID NO: 91] | FQGSHVPWT [SEQ ID NO: 92] |
| | NO: 90] | | |

| | |
|---|---|
| Full V_{H} | QVQLQQPGSVLVRPGASVKLSCKASGFTFATSWMHWAKERPGQGLEWIGQIHPNSGNTNYNEKFKGKA |
| | TLTVDTSSSTAYVDLSSLTSEDSAVYYCARGADYAMDYWGQGTSVTVSS [SEQ ID NO: 93] |
| Full V_{L} | DVLMTQTPLSLPVSLGDQASISCRSSQSIVHSNGNTYLEWYLQKPGQSPKLLIYIVSNRFSGVPDRFS |
| | GSGSGTDFTLKISRVEAEDLGVYYCFQGSHVPWTFGGGTKLEIK [SEQ ID NO: 94] |
| DNA for | CAGGTCCAACTGCAGCAGCCTGGGTCTGTGCTGGTGAGGCCTGGAGCTTCAGTGAAGCTGTCCTGCAA |
| Full V_{H} | GGCTTCTGGCTTCACCTTCGCCACCTCCTGGATGCACTGGGCGAAGGAGAGGCCTGGACAAGGCCTTG |
| | AGTGGATTGGACAGATTCATCCTAATAGTGGTAATACTAACTACAATGAGAAGTTCAAGGGCAAGGCC |
| | ACACTGACTGTAGACACATCCTCCAGCACAGCCTACGTGGATCTCAGCAGCCTGACATCTGAGGACTC |
| | TGCGGTCTATTACTGTGCAAGAGGGGCGGACTATGCTATGGACTACTGGGGTCAAGGAACCTCAGTCA |
| | CCGTCTCCTCA [SEQ ID NO: 95] |
| DNA for | GATGTTTTGATGACCCAAACTCCACTCTCCCTGCCTGTCAGTCTTGGAGATCAAGCCTCCATCTCTTG |
| Full V_{L} | CAGATCTAGTCAGAGCATTGTACATAGTAATGGAAACACCTATTTAGAATGGTACCTGCAGAAACCAG |
| | GCCAGTCTCCAAAGCTCCTGATCTACATAGTTTCCAACCGATTTTCTGGGGTCCCAGACAGGTTCAGT |
| | GGCAGTGGATCAGGGACAGATTTCACACTCAAGATCAGCAGAGTGGAGGCTGAGGATCTGGGAGTTTA |
| | TTACTGCTTTCAAGGTTCACATGTTCCGTGGACGTTCGGTGGAGGCACCAAGCTGGAAATCAAA |
| | [SEQ ID NO: 96] |

### 5.3.2. Monoclonal Antibodies

The presently disclosed subject matter provides antibodies (e.g., human antibodies, e.g., human monoclonal antibodies) that specifically bind to CD40 (e.g., human CD40). The V_{H} amino acid sequences of anti-CD40 antibodies 10-K9, 26-I22, 39-K17, 41-J9, 41-E17, 30-P15, 12-O17, 41-I22, 9-P1, and 10-B8 are set forth in SEQ ID NOs: 14, 24, 34, 41, 49, 59, 68, 78, 83, and 93. The V_{L} amino acid sequences of 10-K9, 26-I22, 39-K17, 41-J9, 41-E17, 30-P15, 12-O17, 41-I22, 9-P1, and 10-B8 are set forth in SEQ ID NOs: 15, 25, 35, 50, 60, 69, 79, 84, and 94.

Given that each of 10-K9, 26-I22, 39-K17, 41-J9, 41-E17, 30-P15, 12-O17, 41-I22, 9-P1, and 10-B8 antibodies can bind to CD40, the V_{H} and V_{L} sequences can be “mixed and matched” to create other anti-CD40 binding molecules. CD40 binding of such “mixed and matched” antibodies can be tested using the binding assays known in the art, including for example, ELISAs, Western blots, RIAs, Biacore analysis. Preferably, when V_{H} and V_{L} chains are mixed and matched, a V_{H} sequence from a particular V_{H}/V_{L} pairing is replaced with a structurally similar V_{H} sequence. Likewise, a V_{L} sequence from a particular V_{H}/V_{L} pairing is replaced with a structurally similar V_{L} sequence.

In certain embodiments, the presently disclosed subject matter provides an antibody or an antigen-binding fragment thereof comprising: (a) a heavy chain variable region (V_{H}) Comprising an amino acid sequence selected from SEQ ID NOs: 14, 24, 34, 41, 49, 59, 68, 78, 83, and 93; and (b) a light chain variable region (V_{L}) comprising an amino acid sequence selected from SEQ ID NOs: 15, 25, 35, 50, 60, 69, 79, 84, and 94; wherein the antibody or antigen-binding fragment specifically binds to CD40, e.g., human CD40. In certain embodiments, the V_{H} and V_{L} are selected from the group consisting of:
(a) a heavy chain variable region comprising the amino acid sequence set forth in SEQ ID NO: 14, and a light chain variable region comprising the amino acid sequence set forth in SEQ ID NO: 15;
(b) a heavy chain variable region comprising the amino acid sequence set forth in SEQ ID NO: 24, and a light chain variable region comprising the amino acid sequence set forth in SEQ ID NO: 25;
(c) a heavy chain variable region comprising the amino acid sequence set forth in SEQ ID NO: 34, and a light chain variable region comprising the amino acid sequence set forth in SEQ ID NO: 35;
(d) a heavy chain variable region comprising the amino acid sequence set forth in SEQ ID NO: 41, and a light chain variable region comprising the amino acid sequence set forth in SEQ ID NO: 35;
(e) a heavy chain variable region comprising the amino acid sequence set forth in SEQ ID NO: 49, and a light chain variable region comprising the amino acid sequence set forth in SEQ ID NO: 50;
(f) a heavy chain variable region comprising the amino acid sequence set forth in SEQ ID NO: 59, and a light chain variable region comprising the amino acid sequence set forth in SEQ ID NO: 60;
(g) a heavy chain variable region comprising the amino acid sequence set forth in SEQ ID NO: 68, and a light chain variable region comprising the amino acid sequence set forth in SEQ ID NO: 69;
(h) a heavy chain variable region comprising the amino acid sequence set forth in SEQ ID NO: 78, and a light chain variable region comprising the amino acid sequence set forth in SEQ ID NO: 79;
(i) a heavy chain variable region comprising the amino acid sequence set forth in SEQ ID NO: 83, and a light chain variable region comprising the amino acid sequence set forth in SEQ ID NO: 84; and
(j) a heavy chain variable region comprising the amino acid sequence set forth in SEQ ID NO: 93, and a light chain variable region comprising the amino acid sequence set forth in SEQ ID NO: 94.

In certain embodiments, the presently disclosed subject matter provides antibodies or antigen-binding fragments thereof that comprise the heavy chain and light chain CDR1s, CDR2s and CDR3s of 10-K9, 26-I22, 39-K17, 41-J9, 41-E17, 30-P15, 12-O17, 41-I22, 9-P1, and 10-B8.

The amino acid sequences of the V_{H} CDR1s of 10-K9, 26-I22, 39-K17, 41-J9, 41-E17, 30-P15, 12-O17, 41-I22, 9-P1, and 10-B8 antibodies are set forth in SEQ ID NOs: 8, 18, 28, 38, 43, 53, 63, 72, and 87. The amino acid sequences of the V_{H} CDR2s of 10-K9, 26-I22, 39-K17, 41-J9, 41-E17, 30-P15, 12-O17, 41-I22, 9-P1, and 10-B8 antibodies are set forth in SEQ ID NOs: 9, 19, 29, 39, 44, 54, 64, 73, and 88. The amino acid sequences of the V_{H} CDR3s of 10-K9, 26-I22, 39-K17, 41-J9, 41-E17, 30-P15, 12-O17, 41-I22, 9-P1, and 10-B8 antibodies are set forth in SEQ ID NOs: 10, 20, 30, 40, 45, 55, 65, 74, 82, and 89.

The amino acid sequences of the V_{L} CDR1s of 10-K9, 26-I22, 39-K17, 41-J9, 41-E17, 30-P15, 12-O17, 41-I22, 9-P1, and 10-B8 antibodies are set forth in SEQ ID NOs: 11, 21, 31, 46, 56, 66, 75, and 90. The amino acid sequences of the V_{L} CDR2s of 10-K9, 26-I22, 39-K17, 41-J9, 41-E17, 30-P15, 12-O17, 41-I22, 9-P1, and 10-B8 antibodies are set forth in SEQ ID NOs: 12, 22, 32, 47, 57, 76, and 91. The amino acid sequences of the V_{L} CDR3s of 10-K9, 26-I22, 39-K17, 41-J9, 41-E17, 30-P15, 12-O17, 41-I22, 9-P1, and 10-B8 antibodies are set forth in SEQ ID NOs: 13, 23, 33, 48, 58, 67, 77, and 92. The CDR regions are delineated using the IMGT system. In certain embodiments, the CDR regions are delineated using the IMGT numbering system accessible at www.imgt.org/IMGT_vquest/input.

Given that each of these antibodies or antigen-binding fragments thereof can bind to CD40 and that antigen-binding specificity is provided primarily by the CDR1, CDR2, and CDR3 regions, the V_{H} CDR1, CDR2, and CDR3 sequences and V_{L} CDR1, CDR2, and CDR3 sequences can be “mixed and matched” (i.e., CDRs from different antibodies can be mixed and match, although each antibody must contain a V_{H} CDR1, CDR2, and CDR3 and a V L CDR1, CDR2, and CDR3) to create other anti-CD40 binding molecules. CD40 binding of such “mixed and matched” antibodies can be tested using the binding assays described above. When V_{H} CDR sequences are mixed and matched, the CDR1, CDR2 and/or CDR3 sequence from a particular V_{H} sequence is replaced with a structurally similar CDR sequence(s). Likewise, when V_{L} CDR sequences are mixed and matched, the CDR1, CDR2 and/or CDR3 sequence from a particular V_{L} sequence preferably is replaced with a structurally similar CDR sequence(s). It will be readily apparent to the ordinarily skilled artisan that novel V_{H} and V_{L} sequences can be created by substituting one or more V_{H} and/or V_{L} CDR region sequences with structurally similar sequences from the CDR sequences of the antibodies or antigen-binding fragments thereof disclosed herein 10-K9, 26-I22, 39-K17, 41-J9, 41-E17, 30-P15, 12-O17, 41-I22, 9-P1, and 10-B8.

In certain embodiments, the presently disclosed subject matter provides an antibody or an antigen-binding fragment thereof comprising:
(a) a heavy chain variable region CDR1 comprising the amino acid sequence set forth in SEQ ID NO: 8, SEQ ID NO: 18, SEQ ID NO: 28, SEQ ID NO: 38, SEQ ID NO: 43, SEQ ID NO: 53, SEQ ID NO: 63, SEQ ID NO: 72, or SEQ ID NO: 87;
(b) a heavy chain variable region CDR2 comprising the amino acid sequence set forth in SEQ ID NO: 9, SEQ ID NO: 19, SEQ ID NO: 29, SEQ ID NO: 39, SEQ ID NO: 44, SEQ ID NO: 54, SEQ ID NO: 64, SEQ ID NO: 73, or SEQ ID NO: 88;
(c) a heavy chain variable region CDR3 comprising the amino acid sequence set forth in SEQ ID NO: 10, SEQ ID NO: 20, SEQ ID NO: 30, SEQ ID NO: 40, SEQ ID NO: 45, SEQ ID NO: 55, SEQ ID NO: 65, SEQ ID NO: 74, SEQ ID NO: 82, or SEQ ID NO: 89;
(d) a light chain variable region CDR1 comprising the amino acid sequence set forth in SEQ ID NO: 11, SEQ ID NO: 21, SEQ ID NO: 31, SEQ ID NO: 46, SEQ ID NO: 56, SEQ ID NO: 66, SEQ ID NO: 75, or SEQ ID NO: 90;
(e) a light chain variable region CDR2 comprising the amino acid sequence set forth in SEQ ID NO: 12, SEQ ID NO: 22, SEQ ID NO: 32, SEQ ID NO: 47, SEQ ID NO: 57, SEQ ID NO: 76, or SEQ ID NO: 91; and
(f) a light chain variable region CDR3 comprising the amino acid sequence set forth in SEQ ID NO: 13, SEQ ID NO: 23, SEQ ID NO: 33, SEQ ID NO: 48, SEQ ID NO: 58, SEQ ID NO: 67, SEQ ID NO: 77, or SEQ ID NO: 92.

In certain embodiments, the antibody or antigen-binding fragment thereof comprises:
(a) a heavy chain variable region CDR1 comprising the amino acid sequence set forth in SEQ ID NO: 8;
(b) a heavy chain variable region CDR2 comprising the amino acid sequence set forth in SEQ ID NO: 9;
(c) a heavy chain variable region CDR3 comprising the amino acid sequence set forth in SEQ ID NO: 10;
(d) a light chain variable region CDR1 comprising the amino acid sequence set forth in SEQ ID NO: 11;
(e) a light chain variable region CDR2 comprising the amino acid sequence set forth in SEQ ID NO: 12; and
(f) a light chain variable region CDR3 comprising the amino acid sequence set forth in SEQ ID NO: 13.

In certain embodiments, the antibody or antigen-binding fragment thereof comprises:
(a) a heavy chain variable region CDR1 comprising the amino acid sequence set forth in SEQ ID NO: 18;
(b) a heavy chain variable region CDR2 comprising the amino acid sequence set forth in SEQ ID NO: 19;
(c) a heavy chain variable region CDR3 comprising the amino acid sequence set forth in SEQ ID NO: 20;
(d) a light chain variable region CDR1 comprising the amino acid sequence set forth in SEQ ID NO: 21;
(e) a light chain variable region CDR2 comprising the amino acid sequence set forth in SEQ ID NO: 22; and
(f) a light chain variable region CDR3 comprising the amino acid sequence set forth in SEQ ID NO: 23.

In certain embodiments, the antibody or antigen-binding fragment thereof comprises:
(a) a heavy chain variable region CDR1 comprising the amino acid sequence set forth in SEQ ID NO: 28;
(b) a heavy chain variable region CDR2 comprising the amino acid sequence set forth in SEQ ID NO: 29;
(c) a heavy chain variable region CDR3 comprising the amino acid sequence set forth in SEQ ID NO: 30;
(d) a light chain variable region CDR1 comprising the amino acid sequence set forth in SEQ ID NO: 31;
(e) a light chain variable region CDR2 comprising the amino acid sequence set forth in SEQ ID NO: 32; and
(f) a light chain variable region CDR3 comprising the amino acid sequence set forth in SEQ ID NO: 33.

In certain embodiments, the antibody or antigen-binding fragment thereof comprises:
(a) a heavy chain variable region CDR1 comprising the amino acid sequence set forth in SEQ ID NO: 38;
(b) a heavy chain variable region CDR2 comprising the amino acid sequence set forth in SEQ ID NO: 39;
(c) a heavy chain variable region CDR3 comprising the amino acid sequence set forth in SEQ ID NO: 40;
(d) a light chain variable region CDR1 comprising the amino acid sequence set forth in SEQ ID NO: 31;
(e) a light chain variable region CDR2 comprising the amino acid sequence set forth in SEQ ID NO: 32; and
(f) a light chain variable region CDR3 comprising the amino acid sequence set forth in SEQ ID NO: 33.

In certain embodiments, the antibody or antigen-binding fragment thereof comprises:
(a) a heavy chain variable region CDR1 comprising the amino acid sequence set forth in SEQ ID NO: 43;
(b) a heavy chain variable region CDR2 comprising the amino acid sequence set forth in SEQ ID NO: 44;
(c) a heavy chain variable region CDR3 comprising the amino acid sequence set forth in SEQ ID NO: 45;
(d) a light chain variable region CDR1 comprising the amino acid sequence set forth in SEQ ID NO: 46;
(e) a light chain variable region CDR2 comprising the amino acid sequence set forth in SEQ ID NO: 47; and
(f) a light chain variable region CDR3 comprising the amino acid sequence set forth in SEQ ID NO: 48.

In certain embodiments, the antibody or antigen-binding fragment thereof comprises:
(a) a heavy chain variable region CDR1 comprising the amino acid sequence set forth in SEQ ID NO: 53;
(b) a heavy chain variable region CDR2 comprising the amino acid sequence set forth in SEQ ID NO: 54;
(c) a heavy chain variable region CDR3 comprising the amino acid sequence set forth in SEQ ID NO: 55;
(d) a light chain variable region CDR1 comprising the amino acid sequence set forth in SEQ ID NO: 56;
(e) a light chain variable region CDR2 comprising the amino acid sequence set forth in SEQ ID NO: 57; and
(f) a light chain variable region CDR3 comprising the amino acid sequence set forth in SEQ ID NO: 58.

In certain embodiments, the antibody or antigen-binding fragment thereof comprises:
(a) a heavy chain variable region CDR1 comprising the amino acid sequence set forth in SEQ ID NO: 63;
(b) a heavy chain variable region CDR2 comprising the amino acid sequence set forth in SEQ ID NO: 64;
(c) a heavy chain variable region CDR3 comprising the amino acid sequence set forth in SEQ ID NO: 65;
(d) a light chain variable region CDR1 comprising the amino acid sequence set forth in SEQ ID NO: 66;
(e) a light chain variable region CDR2 comprising the amino acid sequence set forth in SEQ ID NO: 22; and
(f) a light chain variable region CDR3 comprising the amino acid sequence set forth in SEQ ID NO: 67.

In certain embodiments, the antibody or antigen-binding fragment thereof comprises:
(a) a heavy chain variable region CDR1 comprising the amino acid sequence set forth in SEQ ID NO: 72;
(b) a heavy chain variable region CDR2 comprising the amino acid sequence set forth in SEQ ID NO: 73;
(c) a heavy chain variable region CDR3 comprising the amino acid sequence set forth in SEQ ID NO: 74;
(d) a light chain variable region CDR1 comprising the amino acid sequence set forth in SEQ ID NO: 75;
(e) a light chain variable region CDR2 comprising the amino acid sequence set forth in SEQ ID NO: 76; and
(f) a light chain variable region CDR3 comprising the amino acid sequence set forth in SEQ ID NO: 77.

In certain embodiments, the antibody or antigen-binding fragment thereof comprises:
(a) a heavy chain variable region CDR1 comprising the amino acid sequence set forth in SEQ ID NO: 63;
(b) a heavy chain variable region CDR2 comprising the amino acid sequence set forth in SEQ ID NO: 64;
(c) a heavy chain variable region CDR3 comprising the amino acid sequence set forth in SEQ ID NO: 82;
(d) a light chain variable region CDR1 comprising the amino acid sequence set forth in SEQ ID NO: 66;
(e) a light chain variable region CDR2 comprising the amino acid sequence set forth in SEQ ID NO: 22; and
(f) a light chain variable region CDR3 comprising the amino acid sequence set forth in SEQ ID NO: 67.

In certain embodiments, the antibody or antigen-binding fragment thereof comprises:
(a) a heavy chain variable region CDR1 comprising the amino acid sequence set forth in SEQ ID NO: 87;
(b) a heavy chain variable region CDR2 comprising the amino acid sequence set forth in SEQ ID NO: 88;
(c) a heavy chain variable region CDR3 comprising the amino acid sequence set forth in SEQ ID NO: 89;
(d) a light chain variable region CDR1 comprising the amino acid sequence set forth in SEQ ID NO: 90;
(e) a light chain variable region CDR2 comprising the amino acid sequence set forth in SEQ ID NO: 91; and
(f) a light chain variable region CDR3 comprising the amino acid sequence set forth in SEQ ID NO: 92.

The constant regions/framework regions of the anti-CD40 antibodies disclosed herein can be altered, for example, by amino acid substitution, to modify the properties of the antibody (e.g., to increase or decrease one or more of: antigen binding affinity, Fc receptor binding, antibody carbohydrate, for example, glycosylation, fucosylation etc., the number of cysteine residues, effector cell function, effector cell function, complement function or introduction of a conjugation site).

In certain embodiments, a presently disclosed anti-CD40 antibody is a fully-human antibody, e.g., any one of 10-K9, 26-I22, 39-K17, 41-J9, 41-E17, 30-P15, 12-O17, 41-I22, 9-P1, and 10-B8. Fully-human mAbs, when administered to humans, causing serious side effects, including anaphylaxis and hypersensitivity reactions.

The use of phage display libraries has made it possible to select large numbers of antibody repertoires for unique and rare Abs against very defined epitopes (for more details on phage display see McCafferty et al., Phage antibodies: filamentous phage displaying antibody variable domains. Nature, 348: 552-554.) The rapid identification of human Fab or single chain Fv (scFv) fragments highly specific for tumor antigen-derived peptide-MHC complex molecules has thus become possible. In addition, by engineering full-length monoclonal antibody (mAb) using the Fab fragments, it is possible to directly generate a therapeutic human mAb, bypassing months of time-consuming work, normally needed for developing therapeutic mAbs. The presently disclosed subject matter involves the development of a fully human mAb that recognizes, for example, a human CD40 polypeptide (e.g., a polypeptide having the amino acid sequence set forth in SEQ ID NO: 7) for cancer therapy.

### 5.3.3. Homologous Antibodies

In certain embodiments, a presently disclosed anti-CD40 antibody or antigen-binding fragment thereof comprises heavy and light chain variable regions comprising amino acid sequences that are homologous or identical to the amino acid sequences of the antibodies described herein (e.g., 10-K9, 26-I22, 39-K17, 41-J9, 41-E17, 30-P15, 12-O17, 41-I22, 9-P1, and 10-B8 antibodies), and wherein the antibodies or antigen-binding fragments thereof retain the desired functional properties of the anti-CD40 antibodies or antigen-binding fragments thereof of the presently disclosed subject matter.

For example, the presently disclosed subject matter provides an anti-CD40 antibody or an antigen-binding fragment thereof, comprising a heavy chain variable region and a light chain variable region, wherein:
(a) the heavy chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93; and
(b) the light chain variable region comprises an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

In certain embodiments, the V_{H} and/or V_{L} amino acid sequences can be at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the sequences set forth above. An antibody having V_{H} and V_{L} regions having high (i.e., 80% or greater) homology or identity to the V_{H} and V_{L} regions of the sequences set forth above, can be obtained by mutagenesis (e.g., site-directed or PCR-mediated mutagenesis), followed by testing of the encoded altered antibody for retained function (i.e., the binding affinity) using the binding assays described herein.

In certain embodiments, a presently disclosed anti-CD40 antibody or antigen-binding fragment thereof comprises heavy and light chain constant regions comprising amino acid sequences that are homologous or identical to the amino acid sequences of the antibodies described herein (e.g., 10-K9, 26-I22, 39-K17, 41-J9, 41-E17, 30-P15, 12-O17, 41-I22, 9-P1, and 10-B8 antibodies), and wherein the antibodies or antigen-binding fragments thereof retain the desired functional properties of the anti-CD40 antibodies or antigen-binding fragments thereof of the presently disclosed subject matter.

As used herein, the percent homology between two amino acid sequences is equivalent to the percent identity between the two sequences. The percent identity or homology between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent homology or identity between two amino acid sequences can be determined using the algorithm of E. Meyers and W. Miller (Comput Appl Biosci (1988); 14:11-17) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J Mol Biol (1970); 48:444-453) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the presently disclosed subject matter can further be used as a “query sequence” to perform a search against public databases to, for example, identify related sequences. Such searches can be performed using the XBLAST program (version 2.0) of Altschul et al., J Mol Biol (1990); 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., Nucleic Acids Res (1997); 25(17):3389-3402. When utilizing BLAST and Gapped BLAST programs, the default parameters of the respective programs (e.g., XBLAST and NBLAST) can be used.

### 5.3.4. Antibodies with Conservative Modifications

In certain embodiments, a presently disclosed anti-CD40 antibody or an antigen-binding fragment thereof comprises a heavy chain variable region comprising CDR1, CDR2 and CDR3 sequences and a light chain variable region comprising CDR1, CDR2 and CDR3 sequences, wherein one or more of these CDR sequences comprise specified amino acid sequences based on the preferred antibodies described herein (e.g., 10-K9, 26-I22, 39-K17, 41-J9, 41-E17, 30-P15, 12-O17, 41-I22, 9-P1, and 10-B8 antibodies), or a conservative modification thereof, and wherein the antibodies retain the desired functional properties of the anti-CD40 antibodies or antigen-binding fragments thereof of the presently disclosed subject matter. The presently disclosed subject matter provides an antibody or an antigen-binding fragment thereof, comprising a heavy chain variable region comprising CDR1, CDR2, and CDR3 sequences and a light chain variable region comprising CDR1, CDR2, and CDR3 sequences, wherein:
(a) the heavy chain variable region CDR3 sequence comprises the amino acid sequence selected from SEQ ID NOs: 10, 20, 30, 40, 45, 55, 65, 74, 82, and 89, and conservative modifications thereof;
(b) the light chain variable region CDR3 sequence comprises an amino acid sequence selected from SEQ ID NOs: 13, 23, 33, 48, 58, 67, 77, and 92, and conservative modifications thereof.

In certain embodiments, the heavy chain variable region CDR3 sequence comprises an amino acid sequence selected from SEQ ID NOs: 10, 20, 30, 40, 45, 55, 65, 74, 82, and 89, and conservative modifications thereof; and the light chain variable region CDR3 sequence comprises an amino acid sequence selected from SEQ ID NOs: 13, 23, 33, 48, 58, 67, 77, and 92, and conservative modifications thereof.

In certain embodiments, the heavy chain variable region CDR2 sequence comprises an amino acid sequence selected from SEQ ID NOs: 9, 19, 29, 39, 44, 54, 64, 73, and 88, and conservative modifications thereof; and the light chain variable region CDR2 sequence comprises an amino acid sequence selected from SEQ ID NOs: 12, 22, 32, 47, 57, 76, and 91, and conservative modifications thereof.

In certain embodiments, the heavy chain variable region CDR1 sequence comprises an amino acid sequence selected from SEQ ID NOs: 8, 18, 28, 38, 43, 53, 63, 72, 63, and 87, and conservative modifications thereof; and the light chain variable region CDR1 sequence comprises an amino acid sequence selected from SEQ ID NOs: 11, 21, 31, 46, 56, 66, 75, and 90, and conservative modifications thereof.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the present disclosure by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. Exemplary conservative amino acid substitutions are shown in Table 11. Amino acid substitutions may be introduced into an antibody of interest and the products screened for a desired activity, e.g., retained/improved antigen binding, decreased immunogenicity, or improved ADCC or CDC. In certain embodiments, a sequence disclosed herein, e.g., a CDR sequence, a V_{H} sequence or a V_{L} sequence, can have up to about one, up to about two, up to about three, up to about four, up to about five, up to about six, up to about seven, up to about eight, up to about nine or up to about ten amino acid residues that are modified and/or substituted.

TABLE 11

| Original Residue | Exemplary conservative amino acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

Amino acids may be grouped according to common side-chain properties:
hydrophobic: Norleucine, Met, Ala, Val, Leu, Ile;
neutral hydrophilic: Cys, Ser, Thr, Asn, Gin;
acidic: Asp, Glu;
basic: His, Lys, Arg;
residues that influence chain orientation: Gly, Pro;
aromatic: Trp, Tyr, Phe.

Non-conservative substitutions will entail exchanging a member of one of these classes for another class.

### 5.3.5. Anti-CD40 Antibodies that Cross-Compete for Binding to CD40 with Anti-CD40 Antibodies of the Invention

The presently disclosed subject matter provides antibodies or antigen-binding fragments thereof that cross-compete with any of the disclosed anti-CD40 antibodies for binding to CD40 (e.g., human CD40). For example, and not by way of limitation, the cross-competing antibodies can bind to the same epitope region, e.g., same epitope, adjacent epitope, or overlapping as any of the anti-CD40 antibodies or antigen-binding fragments thereof of the presently disclosed subject matter. In certain embodiments, the reference antibody or reference antigen-binding fragments thereof for cross-competition studies can be any one of the anti-CD40 antibodies or antigen-binding fragments thereof disclosed herein, e.g., 10-K9, 26-I22, 39-K17, 41-J9, 41-E17, 30-P15, 12-O17, 41-I22, 9-P1, and 10-B8 antibodies.

Such cross-competing antibodies can be identified based on their ability to cross-compete with any one of the presently disclosed anti-CD40 antibodies or antigen-binding fragments thereof in standard CD40 binding assays. For example, Biacore analysis, ELISA assays or flow cytometry can be used to demonstrate cross-competition with the antibodies of the presently disclosed subject matter. The ability of a test antibody to inhibit the binding of, for example, any one of the presently disclosed anti-CD40 antibodies (e.g., 10-K9, 26-I22, 39-K17, 41-J9, 41-E17, 30-P15, 12-O17, 41-I22, 9-P1, and 10-B8 antibodies) to CD40 (e.g., human CD40) demonstrates that the test antibody can compete with any one of the presently disclosed anti-CD40 antibodies or antigen-binding fragments thereof for binding to CD40 (e.g., human CD40) and thus binds to the same epitope region on CD40 (e.g., human CD40) as any one of the presently disclosed anti-CD40 antibodies or antigen-binding fragments thereof. In certain embodiments, the cross-competing antibody or antigen-binding fragment thereof binds to the same epitope on CD40 (e.g., human CD40) as any one of the presently disclosed anti-CD40 antibodies or antigen-binding fragments thereof.

### 5.3.6. Characterization of Antibody Binding to Antigen

Antibodies or antigen-binding fragments thereof of the presently disclosed subject can be tested for binding to CD40 by, for example, standard ELISA. To determine if the selected anti-CD40 antibodies bind to unique epitopes, each antibody can be biotinylated using commercially available reagents (Pierce, Rockford, IL). Competition studies using unlabeled monoclonal antibodies and biotinylated monoclonal antibodies can be performed using CD40 coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. Anti-CD40 human IgGs can be further tested for reactivity with CD40 antigen by Western blotting.

In certain embodiments, the K_{D} is measured by a radiolabeled antigen binding assay (RIA). In certain embodiments, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J Mol Biol (1999); 293:865-881).

In certain embodiments, the K_{D} is measured using a BIACORE® surface plasmon resonance assay. For example, an assay using a BIACORE®-2000 or a BIACORE®-3000 (BIAcore, Inc., Piscataway, NJ)

### 5.3.7. Immunoconjugates

The presently disclosed subject provides an anti-CD40 antibody or an antigen-binding fragment thereof, conjugated to a therapeutic moiety, such as a protein, a polynucleotide, or a drug (e.g., an immunostimulator). Such conjugates are referred to herein as “immunoconjugates”.

Anti-CD40 antibodies or antigen-binding fragments thereof of the presently disclosed subject matter also can be conjugated to a radioactive isotope to generate radiopharmaceuticals, also referred to as radioimmunoconjugates. Non-limiting examples of radioactive isotopes that can be conjugated to antibodies for use diagnostically include ⁴⁷Sc, ⁶⁷Cu, ⁹⁰Y, ¹³¹I, ¹⁴⁹Tb, ¹⁶¹Tb, ¹⁷⁷Lu, ²²⁵Ac, ²¹³Bi, ²²³Ra and ²²⁷Th. Methods for preparing radioimmunconjugates are established in the art. Examples of radioimmunoconjugates are commercially available, including Zevalin™ (IDEC Pharmaceuticals) and Bexxar™ (Corixa Pharmaceuticals), and similar methods can be used to prepare radioimmunoconjugates using the presently disclosed anti-CD40 antibodies.

The antibody conjugates of the presently disclosed subject matter can be used to modify a given biological response, and the drug moiety is not to be construed as limited to classical chemical therapeutic agents. For example, the drug moiety may be a protein or polypeptide possessing a desired biological activity. Such proteins may include, for example, a protein such as tumor necrosis factor (TNF) or interferon-γ; or, biological response modifiers such as, for example, lymphokines, interleukin-1 (IL-1), interleukin-2 (IL-2), interleukin-6 (IL-6), granulocyte macrophage colony stimulating factor (GM-CSF), granulocyte colony stimulating factor (G-CSF), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

### 5.3.8. Multi-Specific Molecules

The presently disclosed subject matter provides multi-specific molecules comprising an anti-CD40 antibody, or a fragment thereof, disclosed herein. A presently disclosed or an antigen-binding fragment thereof can be derivatized or linked to one more functional molecules, e.g., one or more peptides or proteins (e.g., one or more antibodies or ligands for a receptor) to generate a multi-specific molecule that binds to two or more different binding sites or target molecules. The presently disclosed anti-CD40 antibody or antigen-binding fragment thereof can in fact be derivatized or linked to more than one other functional molecules to generate multi-specific molecules that bind to more than two different binding sites and/or target molecules. To create a multi-specific molecule, a presently disclosed anti-CD40 antibody or an antigen-binding fragment thereof can be functionally linked (e.g., by chemical coupling, genetic fusion, noncovalent association or otherwise) to one or more other binding molecules, such as another antibody, antibody fragment, peptide or binding mimetic, such that a bispecific molecule.

In certain embodiments, the multi-specific molecule is a bispecific molecule. In certain embodiments, the bispecific molecules comprise at least a first binding specificity for CD40 and a second binding specificity for a second target epitope region. The second target epitope region can be a CD40 epitope, or a non-CD40 epitope, e.g., a different antigen. In certain embodiments, the multi-specific molecule comprises a first binding specificity for CD40, a second binding specificity for a second target, and a third binding specificity for a third target. In certain embodiments, the second target is an antigen expressed on the surface of an immune cell (e.g., a T cell, or a human immune effector cell). In certain embodiments, the multi-specific molecule is capable of recruiting the activity of that immune effector cell by specifically binding to the effector antigen on the human immune effector cell, thereby enhancing effector function. In certain embodiments, the third target is an antigen expressed on a senescent cell.

The multi-specific molecules of the presently disclosed subject matter can be prepared by conjugating the constituent binding specificities using methods known in the art. For example, each binding specificity of the multi-specific molecule can be generated separately and then conjugated to one another. When the binding specificities are proteins or peptides, a variety of coupling or cross-linking agents can be used for covalent conjugation. Non-limiting examples of cross-linking agents include protein A, carbodiimide, N-succinimidyl-S-acetyl-thioacetate (SATA), 5, 5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. (1984) J. Exp. Med. 160:1686; Liu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139: 2367-2375). Conjugating agents can be SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In certain embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the multi-specific molecule is a mAb×mAb, mAb×Fab, Fab×F(ab′)₂ or ligand×Fab fusion protein.

Binding of the multi-specific molecules to their specific targets can be confirmed by, for example, enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest. Alternatively, the complexes can be detected using any of a variety of other immunoassays. For example, the antibody can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography.

### 5.4. Fc Fusion Proteins

The presently disclosed subject matter provides Fc fusion proteins that specifically bind to CD40 (e.g., human CD40). As used herein, the terms “Fc fusion protein” refers to homodimers in which an Fc domain of an antibody is covalently linked to another protein, e.g., an scFv disclosed in Section 5.3.1). Fc is the crystallizable fragment derived from Ig which has five classes including IgG, IgA, IgD, IgM, and IgE in human (Schroeder and Cavacini L, J Allergy Clin Immunol (2010) 125(2 Suppl 2):541-52). Fc plays multiple roles in activation and recruiting of immune leukocytes, triggering of antibody-dependent cell-mediated cytotoxicity (ADCC) and antibody-dependent cell-mediated phagocytosis (Ravetch and Bolland, Annu Rev Immunol (2001) 19:275-90; Woof and Burton, Nat Rev Immunol (2004) 4(2):89-99). In addition, Fc can bind to the serum complement molecule (C1q) to initiate the assembly of membrane attack complex formed by complement cascade proteins to destroy target cells, which is termed complement-dependent cytotoxicity (CDC) (Walport, N Engl J Med (2001) 344(14):1058-66; Walport, N Engl J Med (2001) 344(15):1140-4). Overall, Fc plays important roles in biological and pharmacological properties including, among other functions, increased stability and aggregation resistance, acquired multivalent binding to the target, enhanced Fc-mediated effector functions, extended serum half-life, and modulated immunogenicity. Additional information regarding Fc proteins can be found in Beck and Reichert, MAbs. Vol. 3. No. 5. Taylor & Francis (2011), the content of which is incorporated by reference in its entirety.

In certain embodiments, the presently disclosed Fc fusion protein comprises an Fc fragment and an anti-CD40 scFv disclosed herein (e.g., one disclosed in Section 5.3.1). In certain embodiments, the anti-CD40 scFv comprises (a) a heavy chain variable region (V_{H}) Comprising the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93; and (b) a light chain variable region (V_{L}) comprising the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94. In certain embodiments, the V_{H} and V_{L} are selected from the group consisting of:
(a) a heavy chain variable region comprising the amino acid sequence set forth in SEQ ID NO: 14, and a light chain variable region comprising the amino acid sequence set forth in SEQ ID NO: 15;
(b) a heavy chain variable region comprising the amino acid sequence set forth in SEQ ID NO: 24, and a light chain variable region comprising the amino acid sequence set forth in SEQ ID NO: 25;
(c) a heavy chain variable region comprising the amino acid sequence set forth in SEQ ID NO: 34, and a light chain variable region comprising the amino acid sequence set forth in SEQ ID NO: 35;
(d) a heavy chain variable region comprising the amino acid sequence set forth in SEQ ID NO: 41, and a light chain variable region comprising the amino acid sequence set forth in SEQ ID NO: 35;
(e) a heavy chain variable region comprising the amino acid sequence set forth in SEQ ID NO: 49, and a light chain variable region comprising the amino acid sequence set forth in SEQ ID NO: 50;
(f) a heavy chain variable region comprising the amino acid sequence set forth in SEQ ID NO: 59, and a light chain variable region comprising the amino acid sequence set forth in SEQ ID NO: 60;
(g) a heavy chain variable region comprising the amino acid sequence set forth in SEQ ID NO: 68, and a light chain variable region comprising the amino acid sequence set forth in SEQ ID NO: 69;
(h) a heavy chain variable region comprising the amino acid sequence set forth in SEQ ID NO: 78, and a light chain variable region comprising the amino acid sequence set forth in SEQ ID NO: 79;
(i) a heavy chain variable region comprising the amino acid sequence set forth in SEQ ID NO: 83, and a light chain variable region comprising the amino acid sequence set forth in SEQ ID NO: 84; and
(j) a heavy chain variable region comprising the amino acid sequence set forth in SEQ ID NO: 93, and a light chain variable region comprising the amino acid sequence set forth in SEQ ID NO: 94.

In certain embodiments, the Fc fusion proteins disclosed herein comprise the heavy chain and light chain CDR1s, CDR2s and CDR3s of 10-K9, 26-I22, 39-K17, 41-J9, 41-E17, 30-P15, 12-O17, 41-I22, 9-P1, and 10-B8.

The amino acid sequences of the V_{H} CDR1s of 10-K9, 26-I22, 39-K17, 41-J9, 41-E17, 30-P15, 12-O17, 41-I22, 9-P1, and 10-B8 antibodies are set forth in SEQ ID NOs: 8, 18, 28, 38, 43, 53, 63, 72, 63, and 87, respectively. The amino acid sequences of the V_{H} CDR2s of 10-K9, 26-I22, 39-K17, 41-J9, 41-E17, 30-P15, 12-O17, 41-I22, 9-P1, and 10-B8 antibodies are set forth in SEQ ID NOs: 9, 19, 29, 39, 44, 54, 64, 73, 64, and 88, respectively. The amino acid sequences of the V_{H} CDR3s of 10-K9, 26-I22, 39-K17, 41-J9, 41-E17, 30-P15, 12-O17, 41-I22, 9-P1, and 10-B8 antibodies are set forth in SEQ ID NOs: 10, 20, 30, 40, 45, 55, 65, 74, 82, and 89, respectively.

The amino acid sequences of the V_{L} CDR1s of 10-K9, 26-I22, 39-K17, 41-J9, 41-E17, 30-P15, 12-O17, 41-I22, 9-P1, and 10-B8 antibodies are set forth in SEQ ID NOs: 11, 21, 31, 31, 46, 56, 66, 75, 66, and 90, respectively. The amino acid sequences of the V_{L} CDR2s of 10-K9, 26-I22, 39-K17, 41-J9, 41-E17, 30-P15, 12-O17, 41-I22, 9-P1, and 10-B8 antibodies are set forth in SEQ ID NOs: 12, 22, 32, 32, 47, 57, 22, 76, 22, and 91, respectively. The amino acid sequences of the V_{L} CDR3s of 10-K9, 26-I22, 39-K17, 41-J9, 41-E17, 30-P15, 12-O17, 41-I22, 9-P1, and 10-B8 antibodies are set forth in SEQ ID NOs: 13, 23, 33, 33, 48, 58, 67, 77, 67, and 92, respectively. The CDR regions are delineated using the IMGT system.

In certain embodiments, the anti-CD40 scFv comprised in the presently disclosed Fc fusion proteins comprises:
(a) a heavy chain variable region CDR1 comprising the amino acid sequence set forth in SEQ ID NO: 8, SEQ ID NO: 18, SEQ ID NO: 28, SEQ ID NO: 38, SEQ ID NO: 43, SEQ ID NO: 53, SEQ ID NO: 63, SEQ ID NO: 72, or SEQ ID NO: 87;
(b) a heavy chain variable region CDR2 comprising the amino acid sequence set forth in SEQ ID NO: 9, SEQ ID NO: 19, SEQ ID NO: 29, SEQ ID NO: 39, SEQ ID NO: 44, SEQ ID NO: 54, SEQ ID NO: 64, SEQ ID NO: 73, or SEQ ID NO: 88;
(c) a heavy chain variable region CDR3 comprising the amino acid sequence set forth in SEQ ID NO: 10, SEQ ID NO: 20, SEQ ID NO: 30, SEQ ID NO: 40, SEQ ID NO: 45, SEQ ID NO: 55, SEQ ID NO: 65, SEQ ID NO: 74, SEQ ID NO: 82, or SEQ ID NO: 89;
(d) a light chain variable region CDR1 comprising the amino acid sequence set forth in SEQ ID NO: 11, SEQ ID NO: 21, SEQ ID NO: 31, SEQ ID NO: 46, SEQ ID NO: 56, SEQ ID NO: 66, SEQ ID NO: 75, or SEQ ID NO: 90;
(e) a light chain variable region CDR2 comprising the amino acid sequence set forth in SEQ ID NO: 12, SEQ ID NO: 22, SEQ ID NO: 32, SEQ ID NO: 47, SEQ ID NO: 57, SEQ ID NO: 76, or SEQ ID NO: 91; and
(f) a light chain variable region CDR3 comprising the amino acid sequence set forth in SEQ ID NO: 13, SEQ ID NO: 23, SEQ ID NO: 33, SEQ ID NO: 48, SEQ ID NO: 58, SEQ ID NO: 67, SEQ ID NO: 77, or SEQ ID NO: 92.

In certain embodiments, the anti-CD40 scFv comprised in the presently disclosed Fc fusion proteins comprises:
(a) a heavy chain variable region CDR1 comprising the amino acid sequence set forth in SEQ ID NO: 8;
(b) a heavy chain variable region CDR2 comprising the amino acid sequence set forth in SEQ ID NO: 9;
(c) a heavy chain variable region CDR3 comprising the amino acid sequence set forth in SEQ ID NO: 10;
(d) a light chain variable region CDR1 comprising the amino acid sequence set forth in SEQ ID NO: 11;
(e) a light chain variable region CDR2 comprising the amino acid sequence set forth in SEQ ID NO: 12; and
(f) a light chain variable region CDR3 comprising the amino acid sequence set forth in SEQ ID NO: 13.

In certain embodiments, the anti-CD40 scFv comprised in the presently disclosed Fc fusion proteins comprises:
(a) a heavy chain variable region CDR1 comprising the amino acid sequence set forth in SEQ ID NO: 18;
(b) a heavy chain variable region CDR2 comprising the amino acid sequence set forth in SEQ ID NO: 19;
(c) a heavy chain variable region CDR3 comprising the amino acid sequence set forth in SEQ ID NO: 20;
(d) a light chain variable region CDR1 comprising the amino acid sequence set forth in SEQ ID NO: 21;
(e) a light chain variable region CDR2 comprising the amino acid sequence set forth in SEQ ID NO: 22; and
(f) a light chain variable region CDR3 comprising the amino acid sequence set forth in SEQ ID NO: 23.

In certain embodiments, the anti-CD40 scFv comprised in the presently disclosed Fc fusion proteins comprises:
(a) a heavy chain variable region CDR1 comprising the amino acid sequence set forth in SEQ ID NO: 28;
(b) a heavy chain variable region CDR2 comprising the amino acid sequence set forth in SEQ ID NO: 29;
(c) a heavy chain variable region CDR3 comprising the amino acid sequence set forth in SEQ ID NO: 30;
(d) a light chain variable region CDR1 comprising the amino acid sequence set forth in SEQ ID NO: 31;
(e) a light chain variable region CDR2 comprising the amino acid sequence set forth in SEQ ID NO: 32; and
(f) a light chain variable region CDR3 comprising the amino acid sequence set forth in SEQ ID NO: 33.

In certain embodiments, the anti-CD40 scFv comprised in the presently disclosed Fc fusion proteins comprises:
(a) a heavy chain variable region CDR1 comprising the amino acid sequence set forth in SEQ ID NO: 38;
(b) a heavy chain variable region CDR2 comprising the amino acid sequence set forth in SEQ ID NO: 39;
(c) a heavy chain variable region CDR3 comprising the amino acid sequence set forth in SEQ ID NO: 40;
(d) a light chain variable region CDR1 comprising the amino acid sequence set forth in SEQ ID NO: 31;
(e) a light chain variable region CDR2 comprising the amino acid sequence set forth in SEQ ID NO: 32; and
(f) a light chain variable region CDR3 comprising the amino acid sequence set forth in SEQ ID NO: 33.

In certain embodiments, the anti-CD40 scFv comprised in the presently disclosed Fc fusion proteins comprises:
(a) a heavy chain variable region CDR1 comprising the amino acid sequence set forth in SEQ ID NO: 43;
(b) a heavy chain variable region CDR2 comprising the amino acid sequence set forth in SEQ ID NO: 44;
(c) a heavy chain variable region CDR3 comprising the amino acid sequence set forth in SEQ ID NO: 45;
(d) a light chain variable region CDR1 comprising the amino acid sequence set forth in SEQ ID NO: 46;
(e) a light chain variable region CDR2 comprising the amino acid sequence set forth in SEQ ID NO: 47; and
(f) a light chain variable region CDR3 comprising the amino acid sequence set forth in SEQ ID NO: 48.

In certain embodiments, the anti-CD40 scFv comprised in the presently disclosed Fc fusion proteins comprises:
(a) a heavy chain variable region CDR1 comprising the amino acid sequence set forth in SEQ ID NO: 53;
(b) a heavy chain variable region CDR2 comprising the amino acid sequence set forth in SEQ ID NO: 54;
(c) a heavy chain variable region CDR3 comprising the amino acid sequence set forth in SEQ ID NO: 55;
(d) a light chain variable region CDR1 comprising the amino acid sequence set forth in SEQ ID NO: 56;
(e) a light chain variable region CDR2 comprising the amino acid sequence set forth in SEQ ID NO: 57; and
(f) a light chain variable region CDR3 comprising the amino acid sequence set forth in SEQ ID NO: 58.

In certain embodiments, the anti-CD40 scFv comprised in the presently disclosed Fc fusion proteins comprises:
(a) a heavy chain variable region CDR1 comprising the amino acid sequence set forth in SEQ ID NO: 63;
(b) a heavy chain variable region CDR2 comprising the amino acid sequence set forth in SEQ ID NO: 64;
(c) a heavy chain variable region CDR3 comprising the amino acid sequence set forth in SEQ ID NO: 65;
(d) a light chain variable region CDR1 comprising the amino acid sequence set forth in SEQ ID NO: 66;
(e) a light chain variable region CDR2 comprising the amino acid sequence set forth in SEQ ID NO: 22; and
(f) a light chain variable region CDR3 comprising the amino acid sequence set forth in SEQ ID NO: 67.

In certain embodiments, the anti-CD40 scFv comprised in the presently disclosed Fc fusion proteins comprises:
(a) a heavy chain variable region CDR1 comprising the amino acid sequence set forth in SEQ ID NO: 72;
(b) a heavy chain variable region CDR2 comprising the amino acid sequence set forth in SEQ ID NO: 73;
(c) a heavy chain variable region CDR3 comprising the amino acid sequence set forth in SEQ ID NO: 74;
(d) a light chain variable region CDR1 comprising the amino acid sequence set forth in SEQ ID NO: 75;
(e) a light chain variable region CDR2 comprising the amino acid sequence set forth in SEQ ID NO: 76; and
(f) a light chain variable region CDR3 comprising the amino acid sequence set forth in SEQ ID NO: 77.

In certain embodiments, the anti-CD40 scFv comprised in the presently disclosed Fc fusion proteins comprises:
(a) a heavy chain variable region CDR1 comprising the amino acid sequence set forth in SEQ ID NO: 63;
(b) a heavy chain variable region CDR2 comprising the amino acid sequence set forth in SEQ ID NO: 64;
(c) a heavy chain variable region CDR3 comprising the amino acid sequence set forth in SEQ ID NO: 82;
(d) a light chain variable region CDR1 comprising the amino acid sequence set forth in SEQ ID NO: 66;
(e) a light chain variable region CDR2 comprising the amino acid sequence set forth in SEQ ID NO: 22; and
(f) a light chain variable region CDR3 comprising the amino acid sequence set forth in SEQ ID NO: 67.

In certain embodiments, the anti-CD40 scFv comprised in the presently disclosed Fc fusion proteins comprises:
(a) a heavy chain variable region CDR1 comprising the amino acid sequence set forth in SEQ ID NO: 87;
(b) a heavy chain variable region CDR2 comprising the amino acid sequence set forth in SEQ ID NO: 88;
(c) a heavy chain variable region CDR3 comprising the amino acid sequence set forth in SEQ ID NO: 89;
(d) a light chain variable region CDR1 comprising the amino acid sequence set forth in SEQ ID NO: 90;
(e) a light chain variable region CDR2 comprising the amino acid sequence set forth in SEQ ID NO: 91; and
(f) a light chain variable region CDR3 comprising the amino acid sequence set forth in SEQ ID NO: 92.

In certain embodiments, the Fc fusion protein comprises an Fc domain and a CH1 domain. In certain embodiments, the Fc domain is a human Fc domain. In certain embodiments, the Fc domain is an IgG2 Fc domain. In certain embodiments, the CH1 domain is a human CH1 domain. In certain embodiments, the CH1 domain is an IgG2 CH1 domain. In certain embodiments, the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 122. In certain embodiments, the Fc fusion protein comprises the amino acid sequence set forth in SEQ ID NO: 122. An exemplary nucleotide sequence encoding the amino acid sequence of SEQ ID NO: 122 is set forth in SEQ ID NO: 123. SEQ ID NO: 122 and SEQ ID NO: 123 are provided below:

[SEQ ID NO: 122]
ASTKGPSVFPLAPSSRSTSESTAALGCLVKDYFPEPVTVSWNSGALTSGVHTFPAVLQSSGLYSLSSVVTVPSSNFGTQT
YTCNVDHKPSNTKVDKTVERKCCVECPPCPAPPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAPIEKTISKTKGQPREPQVYTLPPSREEMTKN
QVSLTCLVKGFYPSDIAVEWESNGQPENNYKTTPPMLDSDGSFFLYSKLTVDKSRWQQGNVFSCSVMHEALHNHYTQKSL
SLSPGK
[SEQ ID NO: 123]
GCCAGCACCAAAGGACCCTCCGTTTTCCCACTTGCCCCCTCCAGTAGAAGCACTTCAGAAAGCACCGCGGCGCTTGGGTG
TCTGGTTAAAGATTATTTTCCTGAACCCGTTACCGTCTCCTGGAACAGTGGGGCGCTTACATCTGGTGTGCACACATTCC
CAGCAGTACTCCAGTCATCAGGACTGTACTCATTGAGCTCCGTTGTTACTGTTCCCAGCTCCAACTTTGGAACCCAAACC
TACACCTGCAACGTCGATCATAAGCCGAGCAATACAAAGGTAGATAAAACAGTGGAGCGAAAATGTTGTGTCGAATGCCC
TCCGTGCCCGGCTCCCCCTGTTGCTGGCCCGTCAGTCTTTCTGTTTCCACCAAAACCCAAAGACACTCTCATGATCAGCA
GAACACCTGAGGTAACTTGCGTCGTGGTAGACGTTTCACACGAAGACCCAGAAGTCCAATTCAATTGGTACGTCGATGGG
GTTGAGGTCCACAACGCCAAAACAAAGCCCCGAGAAGAGCAGTTTAACAGCACTTTTAGAGTGGTTAGTGTATTGACGGT
CGTGCACCAAGACTGGCTCAACGGAAAGGAATATAAATGTAAAGTATCAAACAAAGGTCTCCCAGCACCAATTGAAAAAA
CAATTAGCAAGACAAAAGGACAACCGCGAGAACCGCAGGTCTACACTCTGCCTCCTTCAAGGGAGGAGATGACTAAGAAT
CAAGTTTCTCTTACCTGTCTCGTAAAAGGATTCTACCCCTCTGATATAGCGGTGGAGTGGGAGTCCAACGGACAGCCCGA
AAACAACTACAAGACAACCCCCCCAATGCTCGACTCAGACGGGTCTTTTTTCCTTTATTCCAAGTTGACAGTCGATAAGT
CAAGATGGCAACAGGGGAATGTTTTCTCATGCTCCGTAATGCACGAAGCGTTGCATAACCACTATACGCAAAAATCTCTG
AGTCTTTCACCGGGCAAG

In certain embodiments, the Fc fusion protein comprises an Fc domain and a hinge domain. In certain embodiments, the Fc domain is a human Fc domain. In certain embodiments, the Fc domain is an IgG2 Fc domain. In certain embodiments, the hinge domain is a human hinge domain. In certain embodiments, the hinge domain is an IgG2 hinge domain. In certain embodiments, the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 124. In certain embodiments, the Fc fusion protein comprises the amino acid sequence set forth in SEQ ID NO: 124. An exemplary nucleotide sequence encoding the amino acid sequence of SEQ ID NO: 124 is set forth in SEQ ID NO: 125. SEQ ID NO: 124 and SEQ ID NO: 125 are provided below:

[SEQ ID NO: 124]
ERKCCVECPPCPAPPVAGPSVFLFPPKPKDTLMISRTPEVTCVVVDVSHEDPEVQFNWYVDGVEVHNAKTKPREEQFNST
FRVVSVLTVVHQDWLNGKEYKCKVSNKGLPAPIEKTISKTKGQPREPQVYTLPPSREEMTKNQVSLTCLVKGFYPSDIAV
EWESNGQPENNYKTTPPMLDSDGSFFLYSKLTVDKSRWQQGNVFSCSVMHEALHNHYTQKSLSLSPGK
[SEQ ID NO: 125]
GAGCGAAAATGTTGTGTCGAATGCCCTCCGTGCCCGGCTCCCCCTGTTGCTGGCCCGTCAGTCTTTCTGTTTCCACCAAA
ACCCAAAGACACTCTCATGATCAGCAGAACACCTGAGGTAACTTGCGTCGTGGTAGACGTTTCACACGAAGACCCAGAAG
TCCAATTCAATTGGTACGTCGATGGGGTTGAGGTCCACAACGCCAAAACAAAGCCCCGAGAAGAGCAGTTTAACAGCACT
TTTAGAGTGGTTAGTGTATTGACGGTCGTGCACCAAGACTGGCTCAACGGAAAGGAATATAAATGTAAAGTATCAAACAA
AGGTCTCCCAGCACCAATTGAAAAAACAATTAGCAAGACAAAAGGACAACCGCGAGAACCGCAGGTCTACACTCTGCCTC
CTTCAAGGGAGGAGATGACTAAGAATCAAGTTTCTCTTACCTGTCTCGTAAAAGGATTCTACCCCTCTGATATAGCGGTG
GAGTGGGAGTCCAACGGACAGCCCGAAAACAACTACAAGACAACCCCCCCAATGCTCGACTCAGACGGGTCTTTTTTCCT
TTATTCCAAGTTGACAGTCGATAAGTCAAGATGGCAACAGGGGAATGTTTTCTCATGCTCCGTAATGCACGAAGCGTTGC
ATAACCACTATACGCAAAAATCTCTGAGTCTTTCACCGGGCAAG

In certain embodiments, the V_{H} and V_{L} of the anti-CD40 scFv are linked via a first linker. In certain embodiments, the first linker comprises the amino acid sequence set forth in SEQ ID NO: 2.

In certain embodiments, the anti-CD40 scFv and the Fc domain are linked via a second linker. In certain embodiments, the first linker is identical to the second linker. In certain embodiments, the second linker comprises the amino acid sequence set forth in SEQ ID NO: 2.

An exemplary nucleotide sequence encoding the amino acid sequence of SEQ ID NO: 2 is set forth in SEQ ID NO: 126.

In certain embodiments, the Fc fusion protein comprises a signal peptide. In certain embodiments, the signal peptide is an IgG kappa signal peptide. In certain embodiments, the signal peptide comprises the amino acid sequence set forth in SEQ ID NO: 116. An exemplary nucleotide sequence encoding the amino acid sequence of SEQ ID NO: 116 is set forth in SEQ ID NO: 117.

In certain embodiments, the Fc fusion protein comprises a tag. In certain embodiments, the tag is a hemagglutinin (HA) tag. In certain embodiments, the HA tag comprises the amino acid sequence set forth in SEQ ID NO: 127. An exemplary nucleotide sequence encoding the amino acid sequence of SEQ ID NO: 127 is set forth in SEQ ID NO: 128. SEQ ID NO: 126-128 are provided below.

[SEQ ID NO: 126]
GGCGGCGGAGGTAGTGGAGGAGGCGGTTCAGGTGGAGGGGGATCT
[SEQ ID NO: 127]
YPYDVPDYA
[SEQ ID NO: 128]
TACCCATACGACGTACCAGATTACGCT

In certain embodiments, the Fc fusion protein comprises an scFv disclosed in Section 5.3.1. In certain embodiments, the scFv comprises a heavy chain variable region and a light chain variable region. In certain embodiments, the heavy chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10. In certain embodiments, the heavy chain variable region comprises the amino acid sequence set forth in SEQ ID NO: 14. In certain embodiments, the light chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13. In certain embodiments, the light chain variable region comprises the amino acid sequence set forth in SEQ ID NO: 15. In certain embodiments, the heavy chain variable region and the light chain variable region are linked via a first linker. In certain embodiments, the first linker comprises the amino acid sequence set forth in SEQ ID NO: 2. In certain embodiments, the Fc fusion protein comprises an Fc domain. In certain embodiments, the Fc domain comprises the amino acid sequence set forth in SEQ ID NO: 122. In certain embodiments, the scFv and Fc domain are linked via a second linker. In certain embodiments, the second linker comprises the amino acid sequence set forth in SEQ ID NO: 2. In certain embodiments, the Fc fusion protein comprises a signal peptide. In certain embodiments, the signal peptide comprises the amino acid sequence set forth in SEQ ID NO: 116. In certain embodiments, the Fc fusion protein comprises a tag. In certain embodiments, the tag comprises the amino acid sequence set forth in SEQ ID NO: 127.

In certain embodiments, the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 129. In certain embodiments, the Fc fusion protein comprises the amino acid sequence set forth in SEQ ID NO: 129. An exemplary nucleotide sequence encoding the amino acid sequence of SEQ ID NO: 129 is set forth in SEQ ID NO: 130. SEQ ID NO: 129 and SEQ ID NO: 130 are provided below.

[SEQ ID NO: 129]
METDTLLLWVLLLWVPGSTGEVQLVESGGDLVKPGGSLKLSCAASGFTFSSYGMSWVRQTPDKRLEWVATISSDGSHTYY
PDSVKGRFTISRDIAKNTLYLQMSSLKSEDTAMYYCVRQGRYVPSFDVWGAGTTVTVSSGGGGSGGGGSGGGGSDIVLTQ
SPATLSVTPGDSVSLSCRASQSIRNNLHWYQQKSHESPRLLIKDASQSISGTPSRFSGSGSGTDFTLSINSVETEDFGMY
FCHQTNNWPFTFGSGTKLEIKGGGGSGGGGSGGGGSASTKGPSVFPLAPSSRSTSESTAALGCLVKDYFPEPVTVSWNSG
ALTSGVHTFPAVLQSSGLYSLSSVVTVPSSNFGTQTYTCNVDHKPSNTKVDKTVERKCCVECPPCPAPPVAGPSVFLFPP
KPKDTLMISRTPEVTCVVVDVSHEDPEVQFNWYVDGVEVHNAKTKPREEQFNSTFRVVSVLTVVHQDWLNGKEYKCKVSN
KGLPAPIEKTISKTKGQPREPQVYTLPPSREEMTKNQVSLTCLVKGFYPSDIAVEWESNGQPENNYKTTPPMLDSDGSFF
LYSKLTVDKSRWQQGNVFSCSVMHEALHNHYTQKSLSLSPGKYPYDVPDYA
[SEQ ID NO: 130]
ATGGAAACCGATACACTCTTGCTGTGGGTGCTTCTTCTTTGGGTGCCTGGATCTACTGGCGAAGTTCAACTGGTTGAGTC
TGGAGGTGATTTGGTTAAGCCTGGTGGATCTTTGAAGCTTTCATGTGCTGCTAGCGGCTTTACGTTTTCCAGCTATGGAA
TGTCATGGGTGAGACAGACCCCGGACAAGAGGCTCGAGTGGGTTGCGACTATATCAAGCGATGGAAGTCACACCTACTAT
CCTGATTCCGTTAAAGGGAGATTCACTATCTCCAGGGACATCGCCAAGAACACCCTGTATCTGCAGATGTCCTCTCTCAA
ATCAGAGGATACTGCGATGTATTATTGCGTGCGCCAAGGGAGATATGTACCATCTTTCGACGTATGGGGCGCAGGCACCA
CCGTCACCGTAAGCAGCGGCGGCGGAGGTAGTGGAGGAGGCGGTTCAGGTGGAGGGGGATCTGATATTGTCCTGACGCAA
AGTCCGGCGACCCTGTCAGTTACGCCCGGAGACAGCGTTTCTTTGTCATGTAGAGCTAGTCAGTCAATTCGAAATAATCT
TCATTGGTATCAACAAAAGAGCCACGAGTCTCCGCGCTTGTTGATCAAAGACGCTTCCCAGTCAATAAGTGGTACCCCGA
GTCGCTTCTCTGGTTCCGGAAGTGGCACAGACTTTACGCTTTCTATCAACTCCGTAGAGACTGAGGATTTCGGCATGTAT
TTTTGCCATCAAACGAATAATTGGCCTTTCACGTTTGGATCTGGTACTAAACTGGAAATAAAGGGAGGTGGCGGAAGTGG
TGGGGGAGGATCAGGGGGTGGAGGTTCTGCCAGCACCAAAGGACCCTCCGTTTTCCCACTTGCCCCCTCCAGTAGAAGCA
CTTCAGAAAGCACCGCGGCGCTTGGGTGTCTGGTTAAAGATTATTTTCCTGAACCCGTTACCGTCTCCTGGAACAGTGGG
GCGCTTACATCTGGTGTGCACACATTCCCAGCAGTACTCCAGTCATCAGGACTGTACTCATTGAGCTCCGTTGTTACTGT
TCCCAGCTCCAACTTTGGAACCCAAACCTACACCTGCAACGTCGATCATAAGCCGAGCAATACAAAGGTAGATAAAACAG
TGGAGCGAAAATGTTGTGTCGAATGCCCTCCGTGCCCGGCTCCCCCTGTTGCTGGCCCGTCAGTCTTTCTGTTTCCACCA
AAACCCAAAGACACTCTCATGATCAGCAGAACACCTGAGGTAACTTGCGTCGTGGTAGACGTTTCACACGAAGACCCAGA
AGTCCAATTCAATTGGTACGTCGATGGGGTTGAGGTCCACAACGCCAAAACAAAGCCCCGAGAAGAGCAGTTTAACAGCA
CTTTTAGAGTGGTTAGTGTATTGACGGTCGTGCACCAAGACTGGCTCAACGGAAAGGAATATAAATGTAAAGTATCAAAC
AAAGGTCTCCCAGCACCAATTGAAAAAACAATTAGCAAGACAAAAGGACAACCGCGAGAACCGCAGGTCTACACTCTGCC
TCCTTCAAGGGAGGAGATGACTAAGAATCAAGTTTCTCTTACCTGTCTCGTAAAAGGATTCTACCCCTCTGATATAGCGG
TGGAGTGGGAGTCCAACGGACAGCCCGAAAACAACTACAAGACAACCCCCCCAATGCTCGACTCAGACGGGTCTTTTTTC
CTTTATTCCAAGTTGACAGTCGATAAGTCAAGATGGCAACAGGGGAATGTTTTCTCATGCTCCGTAATGCACGAAGCGTT
GCATAACCACTATACGCAAAAATCTCTGAGTCTTTCACCGGGCAAGTACCCATACGACGTACCAGATTACGCTGCGATCG
CAGATCCGGATTAG

In certain embodiments, the Fc fusion protein comprises an scFv disclosed in Section 5.3.1. In certain embodiments, the scFv comprises a heavy chain variable region and a light chain variable region. In certain embodiments, the heavy chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10. In certain embodiments, the heavy chain variable region comprises the amino acid sequence set forth in SEQ ID NO: 14. In certain embodiments, the light chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13. In certain embodiments, the light chain variable region comprises the amino acid sequence set forth in SEQ ID NO: 15. In certain embodiments, the heavy chain variable region and the light chain variable region are linked via a first linker. In certain embodiments, the first linker comprises the amino acid sequence set forth in SEQ ID NO: 2. In certain embodiments, the Fc fusion protein comprises an Fc domain. In certain embodiments, the Fc domain comprises the amino acid sequence set forth in SEQ ID NO: 124. In certain embodiments, the scFv and Fc domain are linked via a second linker. In certain embodiments, the second linker comprises the amino acid sequence set forth in SEQ ID NO: 2. In certain embodiments, the Fc fusion protein comprises a signal peptide. In certain embodiments, the signal peptide comprises the amino acid sequence set forth in SEQ ID NO: 116. In certain embodiments, the Fc fusion protein comprises a tag. In certain embodiments, the tag comprises the amino acid sequence set forth in SEQ ID NO: 127.

In certain embodiments, the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 131. In certain embodiments, the Fc fusion protein comprises the amino acid sequence set forth in SEQ ID NO: 131. An exemplary nucleotide sequence encoding the amino acid sequence of SEQ ID NO: 131 is set forth in SEQ ID NO: 132. SEQ ID NO: 131 and SEQ ID NO: 132 are provided below.

[SEQ ID NO: 131]
METDTLLLWVLLLWVPGSTGEVQLVESGGDLVKPGGSLKLSCAASGFTFSSYGMSWVRQTPDKRLEWVATISSDGSHTYY
PDSVKGRFTISRDIAKNTLYLQMSSLKSEDTAMYYCVRQGRYVPSFDVWGAGTTVTVSSGGGGSGGGGSGGGGSDIVLTQ
SPATLSVTPGDSVSLSCRASQSIRNNLHWYQQKSHESPRLLIKDASQSISGTPSRFSGSGSGTDFTLSINSVETEDFGMY
FCHQTNNWPFTFGSGTKLEIKGGGGSGGGGSGGGGSERKCCVECPPCPAPPVAGPSVFLFPPKPKDTLMISRTPEVTCVV
VDVSHEDPEVQFNWYVDGVEVHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGSFFLYSKLTVDKSRWQQGNVF
SCSVMHEALHNHYTQKSLSLSPGKYPYDVPDYA
[SEQ ID NO: 132]
ATGGAAACCGATACACTCTTGCTGTGGGTGCTTCTTCTTTGGGTGCCTGGATCTACTGGCGAAGTTCAACTGGTTGAGTC
TGGAGGTGATTTGGTTAAGCCTGGTGGATCTTTGAAGCTTTCATGTGCTGCTAGCGGCTTTACGTTTTCCAGCTATGGAA
TGTCATGGGTGAGACAGACCCCGGACAAGAGGCTCGAGTGGGTTGCGACTATATCAAGCGATGGAAGTCACACCTACTAT
CCTGATTCCGTTAAAGGGAGATTCACTATCTCCAGGGACATCGCCAAGAACACCCTGTATCTGCAGATGTCCTCTCTCAA
ATCAGAGGATACTGCGATGTATTATTGCGTGCGCCAAGGGAGATATGTACCATCTTTCGACGTATGGGGCGCAGGCACCA
CCGTCACCGTAAGCAGCGGCGGCGGAGGTAGTGGAGGAGGCGGTTCAGGTGGAGGGGGATCTGATATTGTCCTGACGCAA
AGTCCGGCGACCCTGTCAGTTACGCCCGGAGACAGCGTTTCTTTGTCATGTAGAGCTAGTCAGTCAATTCGAAATAATCT
TCATTGGTATCAACAAAAGAGCCACGAGTCTCCGCGCTTGTTGATCAAAGACGCTTCCCAGTCAATAAGTGGTACCCCGA
GTCGCTTCTCTGGTTCCGGAAGTGGCACAGACTTTACGCTTTCTATCAACTCCGTAGAGACTGAGGATTTCGGCATGTAT
TTTTGCCATCAAACGAATAATTGGCCTTTCACGTTTGGATCTGGTACTAAACTGGAAATAAAGGGAGGTGGCGGAAGTGG
TGGGGGAGGATCAGGGGGTGGAGGTTCTGAGCGAAAATGTTGTGTCGAATGCCCTCCGTGCCCGGCTCCCCCTGTTGCTG
GCCCGTCAGTCTTTCTGTTTCCACCAAAACCCAAAGACACTCTCATGATCAGCAGAACACCTGAGGTAACTTGCGTCGTG
GTAGACGTTTCACACGAAGACCCAGAAGTCCAATTCAATTGGTACGTCGATGGGGTTGAGGTCCACAACGCCAAAACAAA
GCCCCGAGAAGAGCAGTTTAACAGCACTTTTAGAGTGGTTAGTGTATTGACGGTCGTGCACCAAGACTGGCTCAACGGAA
AGGAATATAAATGTAAAGTATCAAACAAAGGTCTCCCAGCACCAATTGAAAAAACAATTAGCAAGACAAAAGGACAACCG
CGAGAACCGCAGGTCTACACTCTGCCTCCTTCAAGGGAGGAGATGACTAAGAATCAAGTTTCTCTTACCTGTCTCGTAAA
AGGATTCTACCCCTCTGATATAGCGGTGGAGTGGGAGTCCAACGGACAGCCCGAAAACAACTACAAGACAACCCCCCCAA
TGCTCGACTCAGACGGGTCTTTTTTCCTTTATTCCAAGTTGACAGTCGATAAGTCAAGATGGCAACAGGGGAATGTTTTC
TCATGCTCCGTAATGCACGAAGCGTTGCATAACCACTATACGCAAAAATCTCTGAGTCTTTCACCGGGCAAGTACCCATA
CGACGTACCAGATTACGCTGCGATCGCAGATCCGGATTAG

In certain embodiments, the Fc fusion protein comprises an scFv disclosed in Section 5.3.1. In certain embodiments, the scFv comprises a heavy chain variable region and a light chain variable region. In certain embodiments, the heavy chain variable region comprises a CDR1 comprising the amino acid sequence set forth in SEQ ID NO: 18, a CDR2 comprising an amino acid sequence set forth in SEQ ID NO: 19, and a CDR3 comprising the amino acid sequence set forth in SEQ ID NO: 20. In certain embodiments, the heavy chain variable region comprises the amino acid sequence set forth in SEQ ID NO: 24. In certain embodiments, the light chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23. In certain embodiments, the light chain variable region comprises the amino acid sequence set forth in SEQ ID NO: 25. In certain embodiments, the heavy chain variable region and the light chain variable region are linked via a first linker. In certain embodiments, the first linker comprises the amino acid sequence set forth in SEQ ID NO: 2. In certain embodiments, the Fc fusion protein comprises an Fc domain. In certain embodiments, the Fc domain comprises the amino acid sequence set forth in SEQ ID NO: 122. In certain embodiments, the scFv and Fc domain are linked via a second linker. In certain embodiments, the second linker comprises the amino acid sequence set forth in SEQ ID NO: 2. In certain embodiments, the Fc fusion protein comprises a signal peptide. In certain embodiments, the signal peptide comprises the amino acid sequence set forth in SEQ ID NO: 116. In certain embodiments, the Fc fusion protein comprises a tag. In certain embodiments, the tag comprises the amino acid sequence set forth in SEQ ID NO: 127.

In certain embodiments, the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 133. In certain embodiments, the Fc fusion protein comprises the amino acid sequence set forth in SEQ ID NO: 133. An exemplary nucleotide sequence encoding the amino acid sequence of SEQ ID NO: 133 is set forth in SEQ ID NO: 134. SEQ ID NO: 133 and SEQ ID NO: 134 are provided below.

[SEQ ID NO: 133]
METDTLLLWVLLLWVPGSTGQVQLQQPGAELVKPGASVKLSCKASGYTFINYWINWMRQRPGQGLEWIGNIYPGSSSTTY
NEKFKSKAALTVDTSSSTAYMQLSSLTSDDSAVYFCARSVGYWYFDVWGAGTTVTVSSGGGGSGGGGSGGGGSDIVMSQS
PSSLAVSVGEKVTLSCKSSQSLFFSGNQKNFLAWYQQKPGQSPNLLIFWASTRESGVPDRFTGSGSGTDFTLTISSVKAE
DLAVYYCQQYYSYPFTFGSGTKLEIKGGGGSGGGGSGGGGSASTKGPSVFPLAPSSRSTSESTAALGCLVKDYFPEPVTV
SWNSGALTSGVHTFPAVLQSSGLYSLSSVVTVPSSNFGTQTYTCNVDHKPSNTKVDKTVERKCCVECPPCPAPPVAGPSV
FLFPPKPKDTLMISRTPEVTCVVVDVSHEDPEVQFNWYVDGVEVHNAKTKPREEQFNSTFRVVSVLTVVHQDWLNGKEYK
CKVSNKGLPAPIEKTISKTKGQPREPQVYTLPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGKYPYDVPDYA
[SEQ ID NO: 134]
ATGGAAACCGATACACTCTTGCTGTGGGTGCTTCTTCTTTGGGTGCCTGGATCTACTGGCCAGGTACAGCTGCAGCAACC
AGGTGCCGAATTGGTCAAACCCGGTGCAAGCGTAAAACTTAGTTGTAAAGCGAGCGGCTACACATTCATCAATTATTGGA
TCAACTGGATGCGACAGCGCCCAGGTCAGGGACTTGAATGGATAGGTAATATTTATCCGGGTTCCTCAAGTACAACGTAC
AACGAGAAATTTAAGTCAAAAGCTGCATTGACCGTGGATACGTCCAGCTCCACTGCGTACATGCAGCTCAGTTCACTTAC
GTCTGATGATTCCGCCGTATATTTCTGCGCTCGGAGCGTGGGTTACTGGTACTTTGACGTATGGGGGGCTGGAACCACAG
TTACGGTTAGTAGCGGCGGCGGAGGTAGTGGAGGAGGCGGTTCAGGTGGAGGGGGATCTGACATAGTTATGTCACAGTCT
CCATCCTCACTTGCCGTCTCAGTCGGCGAAAAGGTAACACTGAGTTGCAAGTCTAGCCAGAGTCTTTTTTTTTCAGGTAA
CCAAAAAAACTTTCTTGCATGGTACCAGCAGAAGCCAGGACAGTCACCGAACCTCCTGATATTTTGGGCAAGCACGCGCG
AGTCAGGTGTACCGGACAGGTTCACCGGTAGTGGCAGCGGAACTGATTTCACACTCACGATAAGTTCCGTGAAGGCTGAG
GATTTGGCTGTGTACTATTGCCAACAATACTATAGCTATCCTTTCACGTTTGGTTCTGGCACGAAGCTGGAAATCAAGGG
AGGTGGCGGAAGTGGTGGGGGAGGATCAGGGGGTGGAGGTTCTGCCAGCACCAAAGGACCCTCCGTTTTCCCACTTGCCC
CCTCCAGTAGAAGCACTTCAGAAAGCACCGCGGCGCTTGGGTGTCTGGTTAAAGATTATTTTCCTGAACCCGTTACCGTC
TCCTGGAACAGTGGGGCGCTTACATCTGGTGTGCACACATTCCCAGCAGTACTCCAGTCATCAGGACTGTACTCATTGAG
CTCCGTTGTTACTGTTCCCAGCTCCAACTTTGGAACCCAAACCTACACCTGCAACGTCGATCATAAGCCGAGCAATACAA
AGGTAGATAAAACAGTGGAGCGAAAATGTTGTGTCGAATGCCCTCCGTGCCCGGCTCCCCCTGTTGCTGGCCCGTCAGTC
TTTCTGTTTCCACCAAAACCCAAAGACACTCTCATGATCAGCAGAACACCTGAGGTAACTTGCGTCGTGGTAGACGTTTC
ACACGAAGACCCAGAAGTCCAATTCAATTGGTACGTCGATGGGGTTGAGGTCCACAACGCCAAAACAAAGCCCCGAGAAG
AGCAGTTTAACAGCACTTTTAGAGTGGTTAGTGTATTGACGGTCGTGCACCAAGACTGGCTCAACGGAAAGGAATATAAA
TGTAAAGTATCAAACAAAGGTCTCCCAGCACCAATTGAAAAAACAATTAGCAAGACAAAAGGACAACCGCGAGAACCGCA
GGTCTACACTCTGCCTCCTTCAAGGGAGGAGATGACTAAGAATCAAGTTTCTCTTACCTGTCTCGTAAAAGGATTCTACC
CCTCTGATATAGCGGTGGAGTGGGAGTCCAACGGACAGCCCGAAAACAACTACAAGACAACCCCCCCAATGCTCGACTCA
GACGGGTCTTTTTTCCTTTATTCCAAGTTGACAGTCGATAAGTCAAGATGGCAACAGGGGAATGTTTTCTCATGCTCCGT
AATGCACGAAGCGTTGCATAACCACTATACGCAAAAATCTCTGAGTCTTTCACCGGGCAAGTACCCATACGACGTACCAG
ATTACGCTGCGATCGCAGATCCGGATTAG

In certain embodiments, the Fc fusion protein comprises an scFv disclosed in Section 5.3.1. In certain embodiments, the scFv comprises a heavy chain variable region and a light chain variable region. In certain embodiments, the heavy chain variable region comprises a CDR1 comprising the amino acid sequence set forth in SEQ ID NO: 18, a CDR2 comprising an amino acid sequence set forth in SEQ ID NO: 19, and a CDR3 comprising the amino acid sequence set forth in SEQ ID NO: 20. In certain embodiments, the heavy chain variable region comprises the amino acid sequence set forth in SEQ ID NO: 24. In certain embodiments, the light chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23. In certain embodiments, the light chain variable region comprises the amino acid sequence set forth in SEQ ID NO: 25. In certain embodiments, the heavy chain variable region and the light chain variable region are linked via a first linker. In certain embodiments, the first linker comprises the amino acid sequence set forth in SEQ ID NO: 2. In certain embodiments, the Fc fusion protein comprises an Fc domain. In certain embodiments, the Fc domain comprises the amino acid sequence set forth in SEQ ID NO: 124. In certain embodiments, the scFv and Fc domain are linked via a second linker. In certain embodiments, the second linker comprises the amino acid sequence set forth in SEQ ID NO: 2. In certain embodiments, the Fc fusion protein comprises a signal peptide. In certain embodiments, the signal peptide comprises the amino acid sequence set forth in SEQ ID NO: 116. In certain embodiments, the Fc fusion protein comprises a tag. In certain embodiments, the tag comprises the amino acid sequence set forth in SEQ ID NO: 127.

In certain embodiments, the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 135. In certain embodiments, the Fc fusion protein comprises the amino acid sequence set forth in SEQ ID NO: 135. An exemplary nucleotide sequence encoding the amino acid sequence of SEQ ID NO: 135 is set forth in SEQ ID NO: 136. SEQ ID NO: 135 and SEQ ID NO: 136 are provided below.

[SEQ ID NO: 135]
METDTLLLWVLLLWVPGSTGQVQLQQPGAELVKPGASVKLSCKASGYTFINYWINWMRQRPGQGLEWIGNIYPGSSSTTY
NEKFKSKAALTVDTSSSTAYMQLSSLTSDDSAVYFCARSVGYWYFDVWGAGTTVTVSSGGGGSGGGGSGGGGSDIVMSQS
PSSLAVSVGEKVTLSCKSSQSLFFSGNQKNFLAWYQQKPGQSPNLLIFWASTRESGVPDRFTGSGSGTDFTLTISSVKAE
DLAVYYCQQYYSYPFTFGSGTKLEIKGGGGSGGGGSGGGGSERKCCVECPPCPAPPVAGPSVFLFPPKPKDTLMISRTPE
VTCVVVDVSHEDPEVQFNWYVDGVEVHNAKTKPREEQFNSTFRVVSVLTVVHQDWLNGKEYKCKVSNKGLPAPIEKTISK
TKGQPREPQVYTLPPSREEMTKNQVSLTCLVKGFYPSDIAVEWESNGQPENNYKTTPPMLDSDGSFFLYSKLTVDKSRWQ
QGNVFSCSVMHEALHNHYTQKSLSLSPGKYPYDVPDYA
[SEQ ID NO: 136]
ATGGAAACCGATACACTCTTGCTGTGGGTGCTTCTTCTTTGGGTGCCTGGATCTACTGGCCAGGTACAGCTGCAGCAACC
AGGTGCCGAATTGGTCAAACCCGGTGCAAGCGTAAAACTTAGTTGTAAAGCGAGCGGCTACACATTCATCAATTATTGGA
TCAACTGGATGCGACAGCGCCCAGGTCAGGGACTTGAATGGATAGGTAATATTTATCCGGGTTCCTCAAGTACAACGTAC
AACGAGAAATTTAAGTCAAAAGCTGCATTGACCGTGGATACGTCCAGCTCCACTGCGTACATGCAGCTCAGTTCACTTAC
GTCTGATGATTCCGCCGTATATTTCTGCGCTCGGAGCGTGGGTTACTGGTACTTTGACGTATGGGGGGCTGGAACCACAG
TTACGGTTAGTAGCGGCGGCGGAGGTAGTGGAGGAGGCGGTTCAGGTGGAGGGGGATCTGACATAGTTATGTCACAGTCT
CCATCCTCACTTGCCGTCTCAGTCGGCGAAAAGGTAACACTGAGTTGCAAGTCTAGCCAGAGTCTTTTTTTTTCAGGTAA
CCAAAAAAACTTTCTTGCATGGTACCAGCAGAAGCCAGGACAGTCACCGAACCTCCTGATATTTTGGGCAAGCACGCGCG
AGTCAGGTGTACCGGACAGGTTCACCGGTAGTGGCAGCGGAACTGATTTCACACTCACGATAAGTTCCGTGAAGGCTGAG
GATTTGGCTGTGTACTATTGCCAACAATACTATAGCTATCCTTTCACGTTTGGTTCTGGCACGAAGCTGGAAATCAAGGG
AGGTGGCGGAAGTGGTGGGGGAGGATCAGGGGGTGGAGGTTCTGAGCGAAAATGTTGTGTCGAATGCCCTCCGTGCCCGG
CTCCCCCTGTTGCTGGCCCGTCAGTCTTTCTGTTTCCACCAAAACCCAAAGACACTCTCATGATCAGCAGAACACCTGAG
GTAACTTGCGTCGTGGTAGACGTTTCACACGAAGACCCAGAAGTCCAATTCAATTGGTACGTCGATGGGGTTGAGGTCCA
CAACGCCAAAACAAAGCCCCGAGAAGAGCAGTTTAACAGCACTTTTAGAGTGGTTAGTGTATTGACGGTCGTGCACCAAG
ACTGGCTCAACGGAAAGGAATATAAATGTAAAGTATCAAACAAAGGTCTCCCAGCACCAATTGAAAAAACAATTAGCAAG
ACAAAAGGACAACCGCGAGAACCGCAGGTCTACACTCTGCCTCCTTCAAGGGAGGAGATGACTAAGAATCAAGTTTCTCT
TACCTGTCTCGTAAAAGGATTCTACCCCTCTGATATAGCGGTGGAGTGGGAGTCCAACGGACAGCCCGAAAACAACTACA
AGACAACCCCCCCAATGCTCGACTCAGACGGGTCTTTTTTCCTTTATTCCAAGTTGACAGTCGATAAGTCAAGATGGCAA
CAGGGGAATGTTTTCTCATGCTCCGTAATGCACGAAGCGTTGCATAACCACTATACGCAAAAATCTCTGAGTCTTTCACC
GGGCAAGTACCCATACGACGTACCAGATTACGCTGCGATCGCAGATCCGGATTAG

### 5.5. Nucleic Acids Encoding the Antibodies, Antigen-Binding Fragments, and/or Fc Fusion Proteins

The presently disclosed subject matter provides nucleic acids encoding the anti-CD40 antibodies or antigen-binding fragments thereof disclosed herein (e.g., those disclosed in Section 5.3). The presently disclosed subject matter provides nucleic acids encoding the heavy chain variable region sequence of any one of the presently disclosed anti-CD40 antibodies or antigen-binding fragments thereof (e.g., 10-K9, 26-I22, 39-K17, 41-J9, 41-E17, 30-P15, 12-O17, 41-I22, 9-P1, and 10-B8 antibodies). In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 16, SEQ ID NO: 26, SEQ ID NO: 36, SEQ ID NO: 42, SEQ ID NO: 51, SEQ ID NO: 61, SEQ ID NO: 70, SEQ ID NO: 80, SEQ ID NO: 85, SEQ ID NO: 95, SEQ ID NO: 118, or SEQ ID NO: 120. In certain embodiments, the nucleic acid comprises or consists of the nucleotide sequence set forth in SEQ ID NO: 16, SEQ ID NO: 26, SEQ ID NO: 36, SEQ ID NO: 42, SEQ ID NO: 51, SEQ ID NO: 61, SEQ ID NO: 70, SEQ ID NO: 80, SEQ ID NO: 85, SEQ ID NO: 95, SEQ ID NO: 118, or SEQ ID NO: 120.

The presently disclosed subject matter provides nucleic acids encoding the light chain variable region sequence of any one of the presently disclosed anti-CD40 antibodies or antigen-binding fragments thereof (e.g., 10-K9, 26-I22, 39-K17, 41-J9, 41-E17, 30-P15, 12-O17, 41-I22, 9-P1, and 10-B8 antibodies). In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 17, SEQ ID NO: 27, SEQ ID NO: 37, SEQ ID NO: 52, SEQ ID NO: 62, SEQ ID NO: 71, SEQ ID NO: 81, SEQ ID NO: 86, SEQ ID NO: 96, SEQ ID NO: 119, or SEQ ID NO: 121. In certain embodiments, the nucleic acid comprises or consists of the nucleotide sequence set forth in SEQ ID NO: 17, SEQ ID NO: 27, SEQ ID NO: 37, SEQ ID NO: 52, SEQ ID NO: 62, SEQ ID NO: 71, SEQ ID NO: 81, SEQ ID NO: 86, SEQ ID NO: 96, SEQ ID NO: 119, or SEQ ID NO: 121.

The presently disclosed subject matter provides nucleic acids encoding the anti-CD40 Fc fusion proteins disclosed herein (e.g., those disclosed in Section 5.4). The presently disclosed subject matter provides nucleic acids encoding the anti-CD40 scFv of any one of the presently disclosed anti-CD40 Fc fusion proteins (e.g., one disclosed in Section 5.4).

The presently disclosed subject matter provides nucleic acids encoding the Fc domain of any one of the presently disclosed anti-CD40 Fc fusion proteins (e.g., one disclosed in Section 5.4). In certain embodiments, the nucleic acid comprises or consists of a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 123 or SEQ ID NO: 125. In certain embodiments, the nucleic acid comprises or consists of the nucleotide sequence set forth in SEQ ID NO: 123 or SEQ ID NO: 125.

Further provided are vectors comprising the presently disclosed nucleic acids. In certain embodiments, the vector is an expression vector. The presently disclosed subject matter further provides host cells comprising the vectors disclosed herein. In certain embodiments, the host cells are T cells.

### 5.6. Cells

The presently disclosed subject matter provides cells comprising a presently disclosed anti-CD40 antibody or antigen-binding fragment thereof (e.g., one disclosed in Section 5.3). The presently disclosed subject matter provides cells comprising a presently disclosed anti-CD40 Fc fusion protein (e.g., one disclosed in Section 5.4). In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell. In certain embodiments, the pluripotent stem cell is an embryonic stem cell (ESC) or an induced pluripotent stem cell (iPSC).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR like fusion molecule. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a Natural Killer (NK) cell. NK cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes. e.g., those disclosed in Sadelain et al., Nat Rev Cancer (2003); 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the a and R heterodimer), in Panelli et al., J Immunol (2000); 164:495-504; Panelli et al., J Immunol (2000); 164:4382-4392 (disclosing lymphocyte cultures derived from tumor infiltrating lymphocytes (TILs) in tumor biopsies), and in Dupont et al., Cancer Res (2005); 65:5417-5427; Papanicolaou et al., Blood (2003); 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

The cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of T cells upon encounter with targeted tumor cells.

In certain embodiments, the cells can be transduced with the presently disclosed anti-CD40 antibody, antigen-binding fragment thereof or the Fc fusion protein such that the cells express the anti-CD40 antibody, antigen-binding fragment thereof, or the Fc fusion protein.

In certain embodiments, the cell further comprises a second soluble antibody, a soluble antigen-binding fragment, or a fusion protein, which binds to a polypeptide having immunosuppressive activity or immunostimulatory activity. The second soluble antibody, antigen-binding fragment, or fusion protein can enhance the immune response of the cell. In certain embodiments, the second soluble antibody, antigen-binding fragment, or fusion protein is secreted from the cell. Non-limiting examples of antibodies, antigen-binding fragments, and fusion proteins include monoclonal antibodies, single-chain variable fragments (scFvs), scFv-Fc fusion proteins, bispecific antibodies, minibodies, and BiTEs. In certain embodiments, the antigen-binding fragment is a scFv. In certain embodiments, the fusion protein is a scFv-Fc fusion protein.

In certain embodiments, the second soluble antibody, antigen-binding fragment, or fusion protein binds to a polypeptide having immunosuppressive activity. In certain embodiments, the polypeptide having immunosuppressive activity is selected from the group consisting of CD47, PD-1, CTLA-4, BTLA, LAG-3, 2B4, CD47, and their corresponding ligands or receptors (including, not limited to, SIRPa, PD-L1, PD-L2, TNFRSF14, CD48, and FGL-1). In certain embodiments, the second soluble antibody, antigen-binding fragment, or fusion protein is an antagonist antibody, antigen-binding fragment, or fusion protein. In certain embodiments, the second soluble antibody, antigen-binding fragment, or fusion protein is an antagonist antibody, antigen-binding fragment, or fusion protein that binds to PD-1. In certain embodiments, the second soluble antibody, antigen-binding fragment, or fusion protein is an antagonist fusion protein that binds to PD-1. In certain embodiments, the second soluble antibody, antigen-binding fragment, or fusion protein is an antagonist scFv that binds to PD-1. In certain embodiments, the second soluble antibody, antigen-binding fragment, or fusion protein is an antagonist antibody, antigen-binding fragment, or fusion protein that binds to CTLA-4. In certain embodiments, the second soluble antibody, antigen-binding fragment, or fusion protein is an antagonist scFv-Fc fusion protein that binds to CTLA-4. In certain embodiments, the second soluble antibody, antigen-binding fragment, or fusion protein is an antagonist scFv that binds to CTLA-4. In certain embodiments, the second soluble antibody, antigen-binding fragment, or fusion protein is an antagonist antibody, antigen-binding fragment, or fusion protein that binds to CD47.

In certain embodiments, the second soluble antibody, antigen-binding fragment, or fusion protein binds to a polypeptide having immunostimulatory activity. In certain embodiments, the polypeptide having immunostimulatory activity is selected from the group consisting of CD28, OX-40, 4-1BB, GITR, and their corresponding ligands or receptors (including, but not limited to, B7-1, B7-2, OX-40L, 4-1BBL, and GITRL). In certain embodiments, the second soluble antibody, antigen-binding fragment, or fusion protein is an agonist antibody, antigen-binding fragment, or fusion protein.

Cells comprising an antigen-recognizing receptor (e.g., a CAR) and a soluble scFv that binds a polypeptide that has immunosuppressive activity or immunostimulatory activity are disclosed in International Patent Publication No. WO 2014/134165, which is incorporated by reference herein in its entirety.

In certain embodiments, the cells comprise a soluble anti-CD40 antibody or antigen-binding fragment disclosed herein (e.g., one disclosed in Section 5.3) and a soluble antibody, antigen-binding fragment, or fusion protein that binds to PD-1. In certain embodiments, the cells comprise a soluble anti-CD40 Fc fusion protein disclosed herein (e.g., one disclosed in Section 5.4) and a soluble antibody, antigen-binding fragment, or fusion protein that binds to PD-1.

In certain embodiments, the presently disclosed cells further comprise an antigen-recognizing receptor that binds to an antigen. In certain embodiments, the antigen-recognizing receptor is a chimeric antigen receptor (CAR). In certain embodiments, the antigen-recognizing receptor is a T-cell receptor (TCR). In certain embodiments, the antigen-recognizing receptor is a TCR like fusion molecule. The antigen-recognizing receptor can bind to a tumor antigen or a pathogen antigen. In certain embodiments, the antigen-recognizing receptor binds to a tumor antigen. In certain embodiments, the tumor antigen is a tumor-specific antigen or a tumor-associated antigen.

### 5.6.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, ERBB, Tyrosinase related protein 1 (TYRP1, gp75), and Delta-like 3 (DLL3).

In certain embodiments, the antigen-recognizing receptor binds to a pathogen antigen, e.g., for use in treating and/or preventing a pathogen infection. Non-limiting examples of pathogens include viruses, bacteria, fungi, parasites, and protozoans capable of causing disease.

Non-limiting examples of pathogenic viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses), human papilloma virus (i.e. HPV), JC virus, Epstein Bar Virus, Merkel cell polyoma virus.

Non-limiting examples of pathogenic bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Clostridium difficile, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 5.6.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains comprising a Variable (V) region and a Constant (C) region. The Constant region is proximal to the cell membrane, followed by a transmembrane region and a short cytoplasmic tail that lacks the ability to transduce a signal. The Variable region binds to the peptide/MHC complex. The variable domain of each pair (alpha/beta or gamma/delta) of TCR polypeptides comprises three complementarity determining regions (CDRs).

In certain embodiments, a TCR can form a receptor complex with three dimeric signaling modules CD3δ/ε, CD3γ/ε and CD3ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the recombinant TCR differs from any naturally occurring TCR by at least one amino acid residue. In certain embodiments, the recombinant TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the recombinant TCR is modified from a naturally occurring TCR by at least one amino acid residue. In certain embodiments, the recombinant TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

In certain embodiments, the TCR recognizes a viral antigen. In certain embodiments, the TCR is expressed in a virus-specific T cell. In certain embodiments, the virus-specific T cell is derived from an individual immune to a viral infection, e.g., BK virus, human herpesvirus 6, Epstein-Barr virus (EBV), cytomegalovirus or adenovirus. In certain embodiments, the virus-specific T cell is a T cell disclosed in Leen et al., Blood, Vol. 121, No. 26, 2013; Barker et al., Blood, Vol. 116, No. 23, 2010; Tzannou et al., Journal of Clinical Oncology, Vol. 35, No. 31, 2017; or Bollard et al., Blood, Vol. 32, No. 8, 2014, each of which is incorporated by reference in its entirety. In certain embodiments, the TCR recognizes a tumor antigen (including a TAA or TSA). In certain embodiments, the TCR is expressed in a tumor-specific T cell. In certain embodiments, the tumor-specific T cell is a tumor-infiltrating T cell generated by culturing T cells with explants of a tumor, e.g., melanoma or an epithelial cancer. In certain embodiments, the tumor-specific T cell is a T cell disclosed in Stevanovic et al, Science, 356, 200-205, 2017; Dudley et al. Journal of Immunotherapy, 26(4): 332-342, 2003; or Goff et al, Journal of Clinical Oncology, Vol. 34, No. 20, 2016, each of which is incorporated by reference in its entirety.

### 5.6.3. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In accordance with the presently disclosed subject matter, a CAR comprises an extracellular antigen-binding domain, a transmembrane domain, and an intracellular signaling domain, wherein the extracellular antigen-binding domain specifically binds to an antigen, e.g., a tumor antigen (TAA or TSA) or a pathogen antigen.

In certain embodiments, the CAR comprises an extracellular antigen-binding domain that binds to GD3, a transmembrane domain, and an intracellular signaling domain. Non-limiting examples of CAR comprising an extracellular antigen-binding domain that binds to GD3 can be found in International Patent Application No. PCT/US2022/22427, which is incorporated by reference in its entirety.

In certain embodiments, the CAR comprises an extracellular antigen-binding domain that binds to TYRP1, a transmembrane domain, and an intracellular signaling domain. Non-limiting examples of CAR comprising an extracellular antigen-binding domain that binds to TYRP1 can be found in International Patent Application No. PCT/US2017/057098, which is incorporated by reference in its entirety.

### 5.6.3.1. Extracellular Antigen-Binding Domain of a CAR

The extracellular antigen-binding domain of the CAR binds to an antigen. In certain embodiments, the antigen is a tumor antigen. In certain embodiments, the antigen is a pathogen antigen. In certain embodiments, the extracellular antigen-binding domain is a single chain variable fragment (scFv). In certain embodiments, the scFv is a human scFv. In certain embodiments, the scFv is a humanized scFv. In certain embodiments, the scFv is a murine scFv. In certain embodiments, the extracellular antigen-binding domain of the CAR is a Fab, which is optionally crosslinked. In certain embodiments, the extracellular antigen-binding domain of the CAR is a F(ab)₂. In certain embodiments, any of the foregoing molecules may be comprised in a fusion protein with a heterologous sequence to form the extracellular antigen-binding domain.

In certain embodiments, the extracellular antigen-binding domain binds to GD3. In certain embodiments, the extracellular antigen-binding domain comprises a V_{H} comprising the amino acid sequence set forth in SEQ ID NO: 137. In certain embodiments, the extracellular antigen-binding domain comprises a V_{L} comprising the amino acid sequence set forth in SEQ ID NO: 138. In certain embodiments, the extracellular antigen-binding domain comprises a V_{H} comprising the amino acid sequence set forth in SEQ ID NO: 137 and a V_{L} comprising the amino acid sequence set forth in SEQ ID NO: 138. SEQ ID NOs: 137 and 138 are provided below.

[SEQ ID NO: 137]
QVQLVESGGGVVQPGRSLRLSCAASGFTFSNFGMHWVRQAPGKGLEWVA
YITSGGSSINYADTVKGRFTISRDNSKNTLYLQMNSLRAEDTAVYYCTR
GGTGTRSLYYFDYWGQGTTVTVSS
[SEQ ID NO: 138]
DIQMTQSPRSLSASVGDRVTITCRASQDIGNFLNWYQQKPGKAPKLLIY
YTSRLQSGVPSRFSGSGSGTDYTLTISSLQPEDFATYYCQQGKTLPYTF
GGGTKVEIKD

In certain embodiments, the extracellular antigen-binding domain comprises a V_{H} comprising a CDR1 comprising the amino acid sequence set forth in SEQ ID NO: 139 or a conservative modification thereof, a CDR2 comprising the amino acid sequence set forth in SEQ ID NO: 140 or a conservative modification thereof, and a CDR3 comprising the amino acid sequence set forth in SEQ ID NO: 141 or a conservative modification thereof. SEQ ID NOs: 139-141 are provided below.

[SEQ ID NO: 139]
NFGMH
[SEQ ID NO: 140]
YITSGGSSINYADTVKG
[SEQ ID NO: 141]
GGTGTRSLYYFDY

In certain embodiments, the extracellular antigen-binding comprises a V_{L} comprising a CDR1 comprising the amino acid sequence set forth in SEQ ID NO: 142 or a conservative modification thereof, a CDR2 comprising the amino acid sequence set forth in SEQ ID NO: 143 or a conservative modification thereof, and a CDR3 comprising the amino acid sequence set forth in SEQ ID NO: 144 or a conservative modification thereof. SEQ ID NOs: 142-144 are provided below.

[SEQ ID NO: 142]
RASQDIGNFLN
[SEQ ID NO: 143]
YTSRLQS
[SEQ ID NO: 144]
QQGKTLPYT

In certain embodiments, the extracellular antigen-binding domain comprises a V_{H} comprising a CDR1 comprising the amino acid sequence set forth in SEQ ID NO: 139 or a conservative modification thereof, a CDR2 comprising the amino acid sequence set forth in SEQ ID NO: 140 or a conservative modification thereof, and a CDR3 comprising the amino acid sequence set forth in SEQ ID NO: 141 or a conservative modification thereof; and a V_{L} comprising a CDR1 comprising the amino acid sequence set forth in SEQ ID NO: 142 or a conservative modification thereof, a CDR2 comprising the amino acid sequence set forth in SEQ ID NO: 143 or a conservative modification, and a CDR3 comprising the amino acid sequence set forth in SEQ ID NO: 144 or a conservative modification thereof. In certain embodiments, the extracellular antigen-binding domain comprises a V_{H} comprising a CDR1 comprising the amino acid sequence set forth in SEQ ID NO: 139 a CDR2 comprising the amino acid sequence set forth in SEQ ID NO: 140, and a CDR3 comprising the amino acid sequence set forth in SEQ ID NO: 141; and a V_{L} comprising a CDR1 comprising the amino acid sequence set forth in SEQ ID NO: 142, a CDR2 comprising the amino acid sequence set forth in SEQ ID NO: 143, and a CDR3 comprising the amino acid sequence set forth in SEQ ID NO: 144.

In certain embodiments, the extracellular antigen-binding domain binds to TYRP1. In certain embodiments, the extracellular antigen-binding domain comprises a V_{H} comprising the amino acid sequence set forth in SEQ ID NO: 145. In certain embodiments, the extracellular antigen-binding domain comprises a V_{L} comprising the amino acid sequence set forth in SEQ ID NO: 146. In certain embodiments, the extracellular antigen-binding domain comprises a V_{H} comprising the amino acid sequence set forth in SEQ ID NO: 145 and a V_{L} comprising the amino acid sequence set forth in SEQ ID NO: 146. SEQ ID NOs: 145 and 146 are provided below.

[SEQ ID NO: 145]
MALPVTALLLPLALLLHAEVQLQQSGAELVRPGALVKLSCKTSGFNIKD
YFLHWVRQRPDQGLEWIGWINPDNGNTVYDPKFQGTASLTADTSSNTVY
LQLSGLTSEDTAVYFCTRRDYTYEKAALDYWGQGASVIVSS
[SEQ ID NO: 146]
AFQMSQSPASLSASVGETVTITCRASGNIYNYLAWYQQKQGKSPHLLVY
DAKTLADGVPSRFSGSGSGTQYSLKISSLQTEDSGNYYCQHFWSLPFTF
GSGTKLEIKAAA

In certain embodiments, the extracellular antigen-binding domain comprises a V_{H} comprising a CDR1 comprising the amino acid sequence set forth in SEQ ID NO: 147 or a conservative modification thereof, a CDR2 comprising the amino acid sequence set forth in SEQ ID NO: 148 or a conservative modification thereof, and a CDR3 comprising the amino acid sequence set forth in SEQ ID NO: 149 or a conservative modification thereof. SEQ ID NOs: 147-149 are provided below.

[SEQ ID NO: 147]
GFNIKDYFLH
[SEQ ID NO: 148]
WINPDNGNTVYDPKFQG
[SEQ ID NO: 149]
DYTYEKAALDY

In certain embodiments, the extracellular antigen-binding comprises a V_{L} comprising a CDR1 comprising the amino acid sequence set forth in SEQ ID NO: 150 or a conservative modification thereof, a CDR2 comprising the amino acid sequence set forth in SEQ ID NO: 151 or a conservative modification thereof, and a CDR3 comprising the amino acid sequence set forth in SEQ ID NO: 152 or a conservative modification thereof. SEQ ID NOs: 150-152 are provided below.

[SEQ ID NO: 150]
RASGNIYNYLA
[SEQ ID NO: 151]
DAKTLAD
[SEQ ID NO: 152]
QHFWSLPFT

In certain embodiments, the extracellular antigen-binding domain comprises a V_{H} comprising a CDR1 comprising the amino acid sequence set forth in SEQ ID NO: 147 or a conservative modification thereof, a CDR2 comprising the amino acid sequence set forth in SEQ ID NO: 148 or a conservative modification thereof, and a CDR3 comprising the amino acid sequence set forth in SEQ ID NO: 149 or a conservative modification thereof; and a V_{L} comprising a CDR1 comprising the amino acid sequence set forth in SEQ ID NO: 150 or a conservative modification thereof, a CDR2 comprising the amino acid sequence set forth in SEQ ID NO: 151 or a conservative modification, and a CDR3 comprising the amino acid sequence set forth in SEQ ID NO: 152 or a conservative modification thereof. In certain embodiments, the extracellular antigen-binding domain comprises a V_{H} comprising a CDR1 comprising the amino acid sequence set forth in SEQ ID NO: 147 a CDR2 comprising the amino acid sequence set forth in SEQ ID NO: 148, and a CDR3 comprising the amino acid sequence set forth in SEQ ID NO: 149; and a V_{L} comprising a CDR1 comprising the amino acid sequence set forth in SEQ ID NO: 150, a CDR2 comprising the amino acid sequence set forth in SEQ ID NO: 151, and a CDR3 comprising the amino acid sequence set forth in SEQ ID NO: 152.

### 5.6.3.2. Transmembrane Domain of a CAR

In certain embodiments, the CAR comprises a transmembrane domain. In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a human CD28 polypeptide (e.g., the transmembrane domain of human CD28 or a portion thereof. In certain embodiments, the CD28 polypeptide comprises or consists of an amino acid sequence that is a consecutive portion of the amino acid sequence having a NCBI Reference No: NP_006130 (SEQ ID NO: 97), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 97. In certain embodiments, the transmembrane domain of the CAR comprises a CD28 polypeptide that comprises or consists of amino acids 153 to 179 of SEQ ID NO: 97. SEQ ID NO: 97 is provided below.

[SEQ ID NO: 97]
MLRLLLALNL FPSIQVTGNK ILVKQSPMLV AYDNAVNLSC
KYSYNLFSRE FRASLHKGLD SAVEVCVVYG NYSQQLQVYS
KTGFNCDGKL GNESVTFYLQ NLYVNQTDIY FCKIEVMYPP
PYLDNEKSNG TIIHVKGKHL CPSPLFPGPS KPFWVLVVVG
GVLACYSLLV TVAFIIFWVR SKRSRLLHSD YMNMTPRRPG
PTRKHYQPYA PPRDFAAYRS

### 5.6.3.3. Hinge/Spacer Region of the CAR

In certain embodiments, the CAR comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 97), a portion of a CD8 polypeptide, a variation of any of the foregoing which is at least about 80%, at least about 85%, at least about 90%, at least about 95%, or at least about 100% homologous or identical thereto, or a synthetic spacer sequence.

In certain embodiments, the hinge/spacer region comprises a native or modified hinge region of a CD28 polypeptide. In certain embodiments, the hinge/spacer region comprises a CD28 polypeptide comprising or consisting of amino acids 114 to 152 of SEQ ID NO: 97.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

### 5.6.3.4. Intracellular Signaling Domain of a CAR

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD3ζ-chain is the primary transmitter of signals from endogenous TCRs.

In certain embodiments, the intracellular signaling domain of the CAR comprises a native CD3. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 98) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 98, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 98. In certain embodiments, the intracellular signaling domain of the CAR comprises a native CD3ζ comprising or consisting of the amino acid sequence of amino acids 52 to 164 of SEQ ID NO: 98. SEQ ID NO: 98 is provided below:

[SEQ ID NO: 98]
MKWKALFTAA ILQAQLPITE AQSFGLLDPK LCYLLDGILF
IYGVILTALF LRVKFSRSAD APAYQQGQNQ LYNELNLGRR
EEYDVLDKRR GRDPEMGGKP QRRKNPQEGL YNELQKDKMA
EAYSEIGMKG ERRRGKGHDG LYQGLSTATK DTYDALHMQA
LPPR

In certain embodiments, the CD3ζ polypeptide comprises or consists of an amino acid sequence that is at least about 85%, about 90%, about 95%, about 96%, about 97%, about 98%, about 99% or about 100% identical or homologous to the amino acid sequence set forth in SEQ ID NO: 153 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 153, which is at least about 20, or at least about 30, or at least about 40, or at least about 50, and up to about 112 amino acids in length. In certain embodiments, the native CD3ζ comprises or consists of the amino acid sequence of amino acids 1 to 112, 1 to 25, 25 to 50, 50 to 100, 50 to 112, or 100 to 112 of SEQ ID NO: 153. In certain embodiments, the intracellular signaling domain of the CAR comprises a CD3ζ polypeptide comprising or consisting of the amino acid sequence set forth in SEQ ID NO: 153. SEQ ID NO: 153 is provided below:

[SEQ ID NO: 153]
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 99.

[SEQ ID NO: 99]
QNQLYNELNLGRREEYDVLDKR

An exemplary nucleotide sequence encoding the amino acid sequence of SEQ ID NO: 99 is set forth in SEQ ID NO: 100, which is provided below.

[SEQ ID NO: 100]
CAGAACCAGCTCTATAACGAGCTCAATCTAGGACGAAGAGAGGAG
TACGATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 101, which is provided below.

[SEQ ID NO: 101]
QNQLFNELNLGRREEFDVLDKR

An exemplary nucleotide sequence encoding the amino acid sequence of SEQ ID NO: 101 is set forth in SEQ ID NO: 102, which is provided below.

[SEQ ID NO: 102]
CAGAACCAGCTCTTTAACGAGCTCAATCTAGGACGAAGAGAGGAG
TTCGATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 103, which is provided below.

[SEQ ID NO: 103]
QEGLYNELQKDKMAEAYSEIGMK

An exemplary nucleotide sequence encoding the amino acid sequence of SEQ ID NO: 103 is set forth in SEQ ID NO: 104, which is provided below.

[SEQ ID NO: 104]
CAGGAAGGCCTGTACAATGAACTGCAGAAAGATAAGATGGCGGAG
GCCTACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 105, which is provided below.

[SEQ ID NO: 105]
QEGLFNELQKDKMAEAFSEIGMK

An exemplary nucleotide sequence encoding the amino acid sequence of SEQ ID NO: 105 is set forth in SEQ ID NO: 106, which is provided below.

[SEQ ID NO: 106]
CAGGAAGGCCTGTTCAATGAACTGCAGAAAGATAAGATGGCGGAG
GCCTTCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 107, which is provided below.

[SEQ ID NO: 107]
HDGLYQGLSTATKDTYDALHMQ

An exemplary nucleotide sequence encoding the amino acid sequence of SEQ ID NO: 107 is set forth in SEQ ID NO: 108, which is provided below.

[SEQ ID NO: 108]
CACGATGGCCTTTACCAGGGTCTCAGTACAGCCACCAAGGACACC
TACGACGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 109, which is provided below.

[SEQ ID NO: 109]
HDGLFQGLSTATKDTFDALHMQ

An exemplary nucleotide sequence encoding the amino acid sequence of SEQ ID NO: 109 is set forth in SEQ ID NO: 110, which is provided below.

[SEQ ID NO: 110]
CACGATGGCCTTTTCCAGGGGCTCAGTACAGCCACCAAGGACACC
TTCGACGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 99, an ITAM2 variant consisting of the amino acid sequence set forth in SEQ ID NO: 105, and an ITAM3 variant consisting of the amino acid sequence set forth in SEQ ID NO: 109. In certain embodiments, the intracellular signaling domain of the CAR is designated as “1XX”. In certain embodiments, the modified CD3ζ polypeptide comprises or consists of the amino acid sequence set forth in SEQ ID NO: 111. SEQ ID NO: 111 is provided below:

[SEQ ID NO: 111]
RVKFSRSADA PAYQQGQNQL YNELNLGRRE EYDVLDKRRG
RDPEMGGKPR RKNPQEGLEN ELQKDKMAEA FSEIGMKGER
RRGKGHDGLF QGLSTATKDT FDALHMQALP PR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 111 or a fragment thereof, and/or may optionally comprise up to one or up to two or up to three conservative amino acid substitutions.

An exemplary nucleotide sequence encoding the amino acid sequence of SEQ ID NO: 111 is set forth in SEQ ID NO: 112, which is provided below.

[SEQ ID NO: 112]
AGAGTGAAGTTCAGCAGGAGCGCAGACGCCCCCGCGTACCAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTTCAAT
GAACTGCAGAAAGATAAGATGGCGGAGGCCTTCAGTGAGATTGGG
ATGAAAGGCGAGCGCCGGAGGGGCAAGGGGCACGATGGCCTTTTC
CAGGGGCTCAGTACAGCCACCAAGGACACCTTCGACGCCCTTCAC
ATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, TNFRSF18 (GITR, CD357), and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the antigen-recognizing receptor comprise or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 97 or a fragment thereof, and/or may optionally comprise up to one or up to two or up to three conservative amino acid substitutions. In certain embodiments, the CD28 polypeptide comprised in the co-stimulatory signaling region comprises or consist of an amino acid sequence that is a consecutive portion of SEQ ID NO: 97, which is at least about 20, or at least about 30, or at least about 40, or at least about 50, and up to about 220 amino acids in length. In certain embodiments, the CD28 polypeptide comprised in the co-stimulatory signaling region comprises or consists of amino acids 1 to 220, 1 to 50, 50 to 100, 100 to 150, 114 to 220, 150 to 200, 180 to 220, or 200 to 220 of SEQ ID NO: 97. In certain embodiments, the intracellular signaling domain of the CAR comprises a co-stimulatory signaling region that comprises a CD28 polypeptide comprising or consisting of amino acids 180 to 220 of SEQ ID NO: 97.

An exemplary nucleotide sequence encoding amino acids 180 to 220 of SEQ ID NO: 97 is set forth in SEQ ID NO: 113, which is provided below.

[SEQ ID NO: 113]
AGGAGTAAGAGGAGCAGGCTCCTGCACAGTGACTACATGAACATG
ACTCCCCGCCGCCCCGGGCCCACCCGCAAGCATTACCAGCCCTAT
GCCCCACCACGCGACTTCGCAGCCTATCGCTCC

In certain embodiments, the intracellular signaling domain of the CAR comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Reference No: NP_031668.3 (or SEQ ID NO: 114) or a fragment thereof, and/or may optionally comprise up to one or up to two or up to three conservative amino acid substitutions. In certain embodiments, the CD28 polypeptide comprised in the co-stimulatory signaling region comprises or consists of an amino acid sequence that is a consecutive portion of SEQ ID NO: 114, which is at least about 20, or at least about 30, or at least about 40, or at least about 50, and up to 218 amino acids in length. In certain embodiments, the CD28 polypeptide comprised in the co-stimulatory signaling region comprises or consists of the amino acid sequence of amino acids 1 to 218, 1 to 50, 50 to 100, 100 to 150, 150 to 218, 178 to 218, or 200 to 218 of SEQ ID NO: 114. In certain embodiments, the co-stimulatory signaling region of the CAR comprises a CD28 polypeptide that comprises or consists of amino acids 178 to 218 of SEQ ID NO: 114. SEQ ID NO: 114 is provided below.

[SEQ ID NO: 114]
MTLRLLFLAL NFFSVQVTEN KILVKQSPLL
VVDSNEVSLS CRYSYNLLAK EFRASLYKGV
NSDVEVCVGN GNFTYQPQFR SNAEFNCDGD
FDNETVTFRL WNLHVNHTDI YFCKIEFMYP
PPYLDNERSN GTIIHIKEKH LCHTQSSPKL
FWALVVVAGV LFCYGLLVTV ALCVIWTNSR
RNRLLQSDYM NMTPRRPGLT RKPYQPYAPA
RDFAAYRP

In certain embodiments, the intracellular signaling domain of the CAR comprises a co-stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Ref. No.: NP_001552 (SEQ ID NO: 115) or a fragment thereof, and/or may optionally comprise up to one or up to two or up to three conservative amino acid substitutions. In certain embodiments, the 4-1BB comprised in the co-stimulatory signaling region comprises or consists of an amino acid sequence that is a consecutive portion of SEQ ID NO: 115, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the 4-1BB polypeptide comprised in the co-stimulatory signaling region comprises or consists of the amino acid sequence of amino acids 1 to 255, 1 to 50, 50 to 100, 100 to 150, 150 to 200, or 200 to 255 of SEQ ID NO: 115. In certain embodiments, the co-stimulatory signaling region comprises a 4-1BB polypeptide comprising or consisting of amino acids 214 to 255 of SEQ ID NO: 115. SEQ ID NO: 115 is provided below.

[SEQ ID NO: 115]
MGNSCYNIVA TLLLVLNFER TRSLQDPCSN CPAGTFCDNN
RNQICSPCPP NSFSSAGGQR TCDICRQCKG VFRTRKECSS
TSNAECDCTP GFHCLGAGCS MCEQDCKQGQ ELTKKGCKDC
CFGTENDQKR GICRPWTNCS LDGKSVLVNG TKERDVVCGP
SPADLSPGAS SVTPPAPARE PGHSPQIISF FLALTSTALL
FLLFFLTLRF SVVKRGRKKL LYIFKQPFMR PVQTTQEEDG
CSCRFPEEEE GGCEL

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, and NKG2D. In certain embodiments, the intracellular signaling domain of the antigen-recognizing receptor comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

### 5.6.4. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

The TCR like fusion molecule can bind to a tumor antigen or a pathogen antigen. In certain embodiments, the TCR like fusion molecule binds to a tumor antigen.

### 5.7. Pharmaceutical Compositions and Methods of Treatment

The presently disclosed subject matter provides compositions comprising a presently disclosed anti-CD40 antibody or an antigen-binding fragment thereof, a presently disclosed Fc fusion protein, a presently disclosed immunoconjugate, a presently disclosed multi-specific molecule. The presently disclosed subject matter also provides compositions comprising a presently disclosed cell or cells (e.g., those disclosed in Section 5.6). In certain embodiments, the composition is a pharmaceutical composition further comprising a pharmaceutically acceptable carrier.

Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the binding proteins. The compositions of the injection can, as is well known in the art, be formulated so as to provide quick, sustained or delayed release of the active ingredient after administration to the mammal.

The presently disclosed subject matter provides various methods of using the anti-CD40 antibodies or antigen-binding fragments thereof, the immunoconjugate, the multi-specific molecule, the Fc fusion proteins, the cells, and the compositions disclosed herein. For example, the presently disclosed subject matter provides methods for treating or ameliorating a disease or disorder in a subject. Non-limiting examples of diseases or disorders include tumors, pathogen infections, autoimmune diseases, and infectious diseases.

In certain embodiments, the method comprises administering one or more of the anti-CD40 antibodies or antigen-binding fragments thereof, the immunoconjugate, the multi-specific molecule, the Fc fusion proteins, the cells, or the composition disclosed herein to the subject. In certain embodiments, the method comprises administering a presently disclosed cell including a presently disclosed anti-CD40 antibody or an antigen-binding fragment thereof, a presently disclosed Fc fusion protein, a presently disclosed immunoconjugate, a presently disclosed multi-specific molecule

In certain embodiments, the disease or disorder is a tumor. In certain embodiments, the presently disclosed cells or composition can reduce tumor burden, induce tumor cell death, reduce the number of tumor cells, reduce tumor size, and/or eradicate the tumor in the subject.

In certain embodiments, the tumor is a solid tumor. In solid tumor immunotherapy, lack of a targetable antigen that is uniformly overexpressed on cancer cells can be overcome by antigen-independent cytotoxic strategies.

In certain embodiments, the tumor is a hematological tumor. Non-limiting examples of hematological tumors include leukemias and lymphomas (e.g., Hodgkin lymphoma, non-Hodgkin's lymphoma, and B-cell lymphomas). In certain embodiments, the tumor is cancer. In certain embodiments, the tumor is hematological malignancy. Non-limiting examples of tumors include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. Suitable tumors further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the tumor (e.g., malignant tumor) is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer (including non-small cell lung cancer (NSCLC) and small cell lung cancer (SCLC), pancreatic cancer, prostate cancer, skin cancer, stomach cancer, glioblastoma, melanoma, sarcomas, and throat cancer.

Non-limiting examples of solid tumors include glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, non-small cell lung cancer (NSCLC), lung adenocarcinoma, stomach adenocarcinoma, cervical and endocervical cancer, kidney clear cell carcinoma, and testicular germ cell tumors.

In certain embodiments, the disease or disorder is a pathogen infection or an infectious disease. In certain embodiments, the disease or disorder is an autoimmune disease.

In certain embodiments, the method further comprises administering to the subject a second therapy. In certain embodiments, the second therapy comprises cyclophosphamide preconditioning, radiation therapy, chemotherapy, a therapy comprising an immune checkpoint inhibitor, or a combination thereof. Non-limiting examples of immune checkpoint inhibitors include anti-PD-L1 antibodies, anti-CTLA-4 antibodies, anti-PD-1 antibodies, anti-LAG3 antibodies, anti-B7-H3 antibodies, anti-TIM3 antibodies, anti-TIGIT antibodies, anti-LAIR1 antibodies, anti-2B4 antibodies, and anti-CD160 antibodies. In certain embodiments, the immune checkpoint inhibitor is an anti-PD-L1 antibody or an anti-PD-1 antibody. The second therapy can be administered to the subject prior to, contemporaneously, or post the administration of the presently disclosed cells or composition. In certain embodiments, the second therapy is administered prior to the administration of the presently disclosed cells or composition. In certain embodiments, the subject is a human.

The presently disclosed subject matter also provides methods of reducing tumor burden in a subject. In certain embodiments, the method comprises administering one or more of the anti-CD40 antibodies or antigen-binding fragments thereof, the immunoconjugate, the multi-specific molecule, the Fc fusion protein, the cells, or the composition disclosed herein to the subject. The presently disclosed anti-CD40 antibodies or antigen-binding fragments thereof, Fc fusion proteins, cells, and compositions can reduce the number of tumor cells, reduce tumor size, and/or eradicate the tumor in the subject.

The presently disclosed subject matter also provides methods of increasing or lengthening survival of a subject having a tumor. In certain embodiments, the method comprises administering one or more of the anti-CD40 antibodies or antigen-binding fragments thereof, the immunoconjugate, the multi-specific molecule, the Fc fusion proteins, the cells, or the composition disclosed herein to the subject. The method can reduce or eradicate tumor burden in the subject.

The presently disclosed subject matter further provides methods for treating and/or preventing a tumor in a subject. In certain embodiments, the method comprises administering one or more of the anti-CD40 antibodies or antigen-binding fragments thereof, the immunoconjugate, the multi-specific molecule, the Fc fusion proteins, the cells, or the composition disclosed herein to the subject.

Such methods comprise administering the presently disclosed anti-CD40 antibodies or antigen-binding fragments thereof, immunoconjugate, multi-specific molecule, Fc fusion proteins, or cells in an amount effective, a presently disclosed composition (e.g., a pharmaceutical composition) to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

Any suitable method or route can be used to administer a presently disclosed anti-CD40 antibody, immunoconjugate, multi-specific molecule, Fc fusion proteins, cells, or compositions, and optionally, to co-administer antineoplastic agents. Routes of administration include, but are not limited to, oral, intravenous, intraperitoneal, subcutaneous, intramuscular, intranodal, intratumoral, intraosseous, intrathecal, pleural, intrapleural, intravesicular, topical, and direct administration. It should be emphasized, however, that the presently disclosed subject matter is not limited to any particular method or route of administration.

The presently disclosed anti-CD40 antibodies or antigen-binding fragments thereof, immunoconjugates, multi-specific molecules, Fc fusion proteins, cells, or compositions can be administered as a conjugate, which binds specifically to the receptor and delivers a toxic, lethal payload following ligand-toxin internalization.

### 5.8. Diagnostic and Prognostic Methods

The presently disclosed anti-CD40 antibodies, antigen-binding fragments thereof, multi-specific molecules, and nucleic acids encoding thereof can be used for diagnostic and prognostic applications as well as use as research tools for detection of CD40 in a biological sample, in a cell, a tissue, or a blood sample. The presently disclosed subject matter provides methods for detecting CD40 in a cell, a tissue, or a blood sample. In certain embodiments, the method comprises: contacting a cell, a tissue, or a blood sample with the anti-CD40 antibody, antigen-binding fragment thereof, or multi-specific molecule disclosed herein, wherein the anti-CD40 antibody, antigen-binding fragment thereof or multi-specific molecule comprises a detectable label; and determining the amount of the labeled anti-CD40 antibody, antigen-binding fragment thereof, or multi-specific molecule bound to the cell, tissue, or blood sample by measuring the amount of detectable label associated with the cell or tissue, wherein the amount of bound anti-CD40 antibody, antigen-binding fragment thereof, or multi-specific molecule indicates the amount of CD40 in the cell, tissue, or a blood sample. The cell or tissue can be any cell or tissue, including any normal, healthy, or cancerous cells and tissues. In certain embodiments, the blood sample is a peripheral blood sample.

The presently disclosed anti-CD40 antibodies or antigen-binding fragments thereof can be used in methods known in the art relating to the localization and/or quantitation of CD40 polypeptides (e.g., for use in measuring levels of the CD40 protein within appropriate physiological samples, for use in diagnostic methods, for use in imaging the polypeptide, and the like). The presently disclosed anti-CD40 antibodies or antigen-binding fragments thereof can be used to isolate a CD40 polypeptide by standard techniques, such as affinity chromatography or immunoprecipitation. The presently disclosed anti-CD40 antibodies or antigen-binding fragments thereof can facilitate the purification of natural immunoreactive

CD40 proteins from biological samples, e.g., mammalian sera or cells as well as recombinantly-produced immunoreactive CD40 proteins expressed in a host system. Moreover, anti-CD40 antibodies of the present technology can be used to detect an immunoreactive CD40 protein (e.g., in plasma, a cellular lysate or cell supernatant) in order to evaluate the abundance and pattern of expression of the immunoreactive polypeptide. The presently disclosed anti-CD40 antibodies or antigen-binding fragments thereof can be used diagnostically to monitor immunoreactive CD40 protein levels in tissue as part of a clinical testing procedure, e.g., to determine the efficacy of a given treatment regimen. As noted above, the detection can be facilitated by coupling (i.e., physically linking) the presently disclosed anti-CD40 antibodies or antigen-binding fragments thereof to a detectable substance.

An exemplary method for detecting the presence or absence of an immunoreactive CD40 protein in a biological sample comprises contacting a biological sample from a subject with a presently disclosed anti-CD40 antibody or an antigen-binding fragment thereof, wherein the presence of an immunoreactive CD40 protein is detected in the biological sample. Detection may be accomplished by means of a detectable label attached to the antibody.

The term “labeled” with regard to the anti-CD40 antibody or antigen-binding fragment thereof is intended to encompass direct labeling of the antibody by coupling (i.e., physically linking) a detectable substance to the antibody, as well as indirect labeling of the antibody by reactivity with another compound that is directly labeled, such as a secondary antibody. Examples of indirect labeling include detection of a primary antibody using a fluorescently-labeled secondary antibody and end-labeling of a DNA probe with biotin such that it can be detected with fluorescently-labeled streptavidin.

In certain embodiments, the presently disclosed anti-CD40 antibodies or antigen-binding fragments thereof are conjugated to one or more detectable labels. For such uses, the presently disclosed anti-CD40 antibodies or antigen-binding fragments thereof may be detectably labeled by covalent or non-covalent attachment of a chromogenic, enzymatic, radioisotopic, isotopic, fluorescent, toxic, chemiluminescent, nuclear magnetic resonance contrast agent or other label.

The presently disclosed detection methods can be used to detect an immunoreactive CD40 protein in a biological sample in vitro as well as in vivo. Non-limiting examples of in vitro techniques for detection of an immunoreactive CD40 protein include enzyme linked immunosorbent assays (ELISAs), Western blots, immunoprecipitations, radioimmunoassay, and immunofluorescence. Furthermore, in vivo techniques for detection of an immunoreactive CD40 protein include introducing into a subject a labeled anti-CD40 antibody or an antigen-binding fragment thereof. For example, the anti-CD40 antibody or antigen-binding fragment thereof can be labeled with a radioactive marker whose presence and location in a subject can be detected by standard imaging techniques. In certain embodiments, the biological sample comprises CD40 protein molecules from the test subject.

The presently disclosed anti-CD40 antibodies or antigen-binding fragments thereof can be used to assay immunoreactive CD40 protein levels in a biological sample (e.g., human plasma) using antibody-based techniques. For example, protein expression in tissues can be studied with classical immunohistological methods. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I, ¹³¹I), carbon (¹⁴C), sulfur (³⁵S), tritium (³H), indium (¹¹¹In), and technetium (^{99m}Tc), and fluorescent labels, such as fluorescein, rhodamine, and green fluorescent protein (GFP), as well as biotin.

In addition to assaying immunoreactive CD40 protein levels in a biological sample, the presently disclosed anti-CD40 antibodies or antigen-binding fragments thereof may be used for in vivo imaging of CD40. Antibodies useful for this method include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the anti-CD40 antibodies by labeling of nutrients for the relevant scFv clone.

The presently disclosed anti-CD40 antibodies or antigen-binding fragments thereof, which are labeled with an appropriate detectable imaging moiety (such as a radioisotope (e.g., ¹³¹I, ¹¹¹IN ^{99m}Tc, ¹⁸F, ⁸⁹Zr), a radio-opaque substance, or a material detectable by nuclear magnetic resonance) are introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled anti-CD40 antibody or antigen-binding fragment thereof then accumulates at the location of cells which contain the specific target polypeptide. For example, the labeled anti-CD40 antibodies or antigen-binding fragments thereof accumulate within the subject in cells and tissues in which the CD40 protein has localized.

Thus, the presently disclosed subject matter provides diagnostic methods of a medical condition. In certain embodiments, the method comprises: (a) assaying the expression of immunoreactive CD40 protein by measuring binding of a presently disclosed anti-CD40 antibody or an antigen-binding fragment thereof in cells or body fluid of an individual; and (b) comparing the amount of immunoreactive CD40 protein present in the sample with a standard reference, wherein an increase or decrease in immunoreactive CD40 protein levels compared to the standard is indicative of a medical condition.

Furthermore, the presently disclosed anti-CD40 antibodies or antigen-binding fragments thereof may be used to purify immunoreactive CD40 protein from a sample. In certain embodiments, the antibodies are immobilized on a solid support. Non-limiting examples of such solid supports include plastics such as polycarbonate, complex carbohydrates such as agarose and sepharose, acrylic resins and such as polyacrylamide and latex beads. Techniques for coupling antibodies to such solid supports are well known in the art.

The simplest method to bind the antigen to the antibody-support matrix is to collect the beads in a column and pass the antigen solution down the column. The efficiency of this method depends on the contact time between the immobilized antibody and the antigen, which can be extended by using low flow rates. The immobilized antibody captures the antigen as it flows past. Alternatively, an antigen can be contacted with the antibody-support matrix by mixing the antigen solution with the support (e.g., beads) and rotating the slurry, allowing maximum contact between the antigen and the immobilized antibody. After the binding reaction has been completed, the slurry is passed into a column for collection of the beads. The beads are washed using a suitable washing buffer and then the pure or substantially pure antigen is eluted.

An antibody or polypeptide of interest can be conjugated to a solid support, such as a bead. In addition, a first solid support such as a bead can also be conjugated, if desired, to a second solid support, which can be a second bead or other support, by any suitable means, including those disclosed herein for conjugation of a polypeptide to a support. Accordingly, any of the conjugation methods and means disclosed herein with reference to conjugation of a polypeptide to a solid support can also be applied for conjugation of a first support to a second support, where the first and second solid support can be the same or different.

Appropriate linkers, which can be cross-linking agents, for use for conjugating a polypeptide to a solid support include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. (Brown et al., Mol. Divers, pp, 4-12 (1995); Rothschild et al., Nucl. Acids Res., 24:351-66 (1996); and U.S. Pat. No. 5,643,722). Other cross-linking reagents are well-known in the art. (See, e.g., Wong (1991), supra; and Hermanson (1996), supra).

An antibody or polypeptide can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g., to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoroacetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

Hydrophobic trityl linkers can also be exploited as acid-labile linkers by using a volatile acid or an appropriate matrix solution, e.g., a matrix solution containing 3-HPA, to cleave an amino linked trityl group from the polypeptide. Acid lability can also be changed. For example, trityl, monomethoxytrityl, dimethoxytrityl or trimethoxytrityl can be changed to the appropriate p-substituted, or more acid-labile tritylamine derivatives, of the polypeptide, i.e., trityl ether and tritylamine bonds can be made to the polypeptide. Accordingly, a polypeptide can be removed from a hydrophobic linker, e.g., by disrupting the hydrophobic attraction or by cleaving tritylether or tritylamine bonds under acidic conditions, including, if desired, under typical MS conditions, where a matrix, such as 3-HPA acts as an acid.

Orthogonally cleavable linkers can also be useful for binding a first solid support, e.g., a bead to a second solid support, or for binding a polypeptide of interest to a solid support. Using such linkers, a first solid support, e.g., a bead, can be selectively cleaved from a second solid support, without cleaving the polypeptide from the support; the polypeptide then can be cleaved from the bead at a later time. For example, a disulfide linker, which can be cleaved using a reducing agent, such as DTT, can be employed to bind a bead to a second solid support, and an acid cleavable bi-functional trityl group could be used to immobilize a polypeptide to the support. As desired, the linkage of the polypeptide to the solid support can be cleaved first, e.g., leaving the linkage between the first and second support intact. Trityl linkers can provide a covalent or hydrophobic conjugation and, regardless of the nature of the conjugation, the trityl group is readily cleaved in acidic conditions.

For example, a bead can be bound to a second support through a linking group which can be selected to have a length and a chemical nature such that high density binding of the beads to the solid support, or high density binding of the polypeptides to the beads, is promoted. Such a linking group can have, e.g., “tree-like” structure, thereby providing a multiplicity of functional groups per attachment site on a solid support. Examples of such linking group; include polylysine, polyglutamic acid, penta-erythrole and tris-hydroxy-aminomethane.

Noncovalent Binding Association. An antibody or polypeptide can be conjugated to a solid support, or a first solid support can also be conjugated to a second solid support, through a noncovalent interaction. For example, a magnetic bead made of a ferromagnetic material, which is capable of being magnetized, can be attracted to a magnetic solid support, and can be released from the support by removal of the magnetic field. Alternatively, the solid support can be provided with an ionic or hydrophobic moiety, which can allow the interaction of an ionic or hydrophobic moiety, respectively, with a polypeptide, e.g., a polypeptide containing an attached trityl group or with a second solid support having hydrophobic character.

A solid support can also be provided with a member of a specific binding pair and, therefore, can be conjugated to a polypeptide or a second solid support containing a complementary binding moiety. For example, a bead coated with avidin or with streptavidin can be bound to a polypeptide having a biotin moiety incorporated therein, or to a second solid support coated with biotin or derivative of biotin, such as iminobiotin.

It should be recognized that any of the binding members disclosed herein or otherwise known in the art can be reversed. Thus, biotin, e.g., can be incorporated into either a polypeptide or a solid support and, conversely, avidin or other biotin binding moiety would be incorporated into the support or the polypeptide, respectively. Other specific binding pairs contemplated for use herein include, but are not limited to, hormones and their receptors, enzyme, and their substrates, a nucleotide sequence and its complementary sequence, an antibody and the antigen to which it interacts specifically, and other such pairs knows to those skilled in the art.

The presently disclosed anti-CD40 antibodies or antigen-binding fragments thereof are useful in diagnostic methods. As such, the presently disclosed subject matter provides methods using the presently disclosed anti-CD40 antibodies or antigen-binding fragments thereof in diagnosis of CD40 activity in a subject. The presently disclosed anti-CD40 antibodies or antigen-binding fragments thereof may be selected such that they have any level of epitope binding specificity and high binding affinity to a CD40 polypeptide.

The presently disclosed anti-CD40 antibodies or antigen-binding fragments thereof can be used to detect an immunoreactive CD40 protein in a variety of standard assay formats. Such formats include immunoprecipitation, Western blotting, ELISA, radioimmunoassay, and immunometric assays. Biological samples can be obtained from any tissue or body fluid of a subject. In certain embodiments, the subject is at an early stage of cancer. In certain embodiments, the early stage of cancer is determined by the level or expression pattern of CD40 protein in a sample obtained from the subject. In certain embodiments, the sample is selected from the group consisting of urine, blood, serum, plasma, saliva, amniotic fluid, cerebrospinal fluid (CSF), and biopsied body tissue.

Immunometric or sandwich assays are one format for the diagnostic methods of the present technology. Such assays use one antibody, e.g., the anti-CD40 antibody or a population of anti-CD40 antibodies immobilized to a solid phase, and another anti-CD40 antibody or a population of anti-CD40 antibodies in solution. Typically, the solution anti-CD40 antibody or population of anti-CD40 antibodies is labeled. If an antibody population is used, the population can contain antibodies binding to different epitope specificities within the target polypeptide. Accordingly, the same population can be used for both solid phase and solution antibody. If anti-CD40 monoclonal antibodies are used, first and second CD40 monoclonal antibodies having different binding specificities are used for the solid and solution phase. Solid phase (also referred to as “capture”) and solution (also referred to as “detection”) antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the CD40 protein with the anti-CD40 antibody, a sample is incubated for a period that usually varies from about 10 min to about 24 hr and is usually about 1 hr. A wash step is then performed to remove components of the sample not specifically bound to the anti-CD40 antibody being used as a diagnostic reagent. When solid phase and solution antibodies are bound in separate steps, a wash can be performed after either or both binding steps. After washing, binding is quantified, typically by detecting a label linked to the solid phase through binding of labeled solution antibody. Usually for a given pair of antibodies or populations of antibodies and given reaction conditions, a calibration curve is prepared from samples containing known concentrations of target antigen. Concentrations of the immunoreactive CD40 protein in samples being tested are then read by interpolation from the calibration curve (i.e., standard curve). Analyte can be measured either from the amount of labeled solution antibody bound at equilibrium or by kinetic measurements of bound labeled solution antibody at a series of time points before equilibrium is reached. The slope of such a curve is a measure of the concentration of the CD40 protein in a sample.

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J.), and the like. Immobilization can be by absorption or by covalent attachment. Optionally, anti-CD40 antibodies can be joined to a linker molecule, such as biotin for attachment to a surface bound linker, such as avidin.

In certain embodiments, the presently disclosed anti-CD40 antibody or antigen-binding fragment thereof is conjugated to a diagnostic agent. The diagnostic agent may comprise a radioactive or non-radioactive label, a contrast agent (such as for magnetic resonance imaging, computed tomography or ultrasound), and the radioactive label can be a gamma-, beta-, alpha-, Auger electron-, or positron-emitting isotope. A diagnostic agent is a molecule which is administered conjugated to an antibody moiety, i.e., antibody or antibody fragment, or sub-fragment, and is useful in diagnosing or detecting a disease by locating the cells comprising the antigen.

Useful diagnostic agents include, but are not limited to, radioisotopes, dyes (such as with the biotin-streptavidin complex), contrast agents, fluorescent compounds or molecules and enhancing agents (e.g., paramagnetic ions) for magnetic resonance imaging (MRI). In certain embodiments, the diagnostic agents are selected from the group consisting of radioisotopes, enhancing agents for use in magnetic resonance imaging, and fluorescent compounds. Chelates may be coupled to the presently disclosed anti-CD40 antibodies or antigen-binding fragments thereof using standard chemistries. The chelate is normally linked to the antibody by a group which enables formation of a bond to the molecule with minimal loss of immunoreactivity and minimal aggregation and/or internal cross-linking.

### 5.9. Kits

The presently disclosed subject matter provides kits for treatment or ameliorating a disease or disorder. In certain embodiments, the kit comprises the anti-CD40 antibodies or antigen-binding fragments thereof, the immunoconjugates, the multi-specific molecules, the Fc fusion proteins, the cells, or the compositions disclosed herein. In certain embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kit further comprises instructions for administering the anti-CD40 antibodies or antigen-binding fragments thereof, the immunoconjugates, the multi-specific molecules, the Fc fusion proteins, the cells, or the compositions disclosed herein to a subject in need the treatment. The instructions can generally include information about the use of the anti-CD40 antibodies or antigen-binding fragments thereof, the immunoconjugates, the multi-specific molecules, the Fc fusion proteins, the cells, and the compositions disclosed herein for the treatment or ameliorating a disease or disorder. In certain embodiments, the instructions include at least one of the following: description of the therapeutic agent; dosage schedule and administration for treatment and/or prevention of a tumor or neoplasm or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

A1. In certain non-limiting embodiments, the presently disclosed subject matter provides an anti-CD40 antibody or an antigen-binding fragment thereof, comprising a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93.

A2. In certain non-limiting embodiments, the presently disclosed subject matter provides an anti-CD40 antibody or an antigen-binding fragment thereof, comprising a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

A3. In certain non-limiting embodiments, the presently disclosed subject matter provides an anti-CD40 antibody or an antigen-binding fragment thereof, comprising
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93; and
(b) a light chain variable region comprising an amino acid sequence that is at least about 80%, at least about 85%, at least about 90%, at least about 95%, at least about 96%, at least about 97%, at least about 98%, at least about 99% at least about 100% homologous or identical to the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

A4. In certain non-limiting embodiments, the presently disclosed subject matter provides an anti-CD40 antibody or an antigen-binding fragment thereof, comprising a heavy chain variable region and a light chain variable region, wherein the heavy chain variable region and the light chain variable region are selected from the group consisting of:
(a) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 14, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 15;
(b) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 24, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 25;
(c) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 34, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35;
(d) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 41, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 35;
(e) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 49, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 50;
(f) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 59, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 60;
(g) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 68, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 69;
(h) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 78, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 79;
(i) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 83, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 84; and
(j) a heavy chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 93, and a light chain variable region comprising an amino acid sequence that is at least about 80%, at least about 85%, at least about 90%, at least about 95%, at least about 96%, at least about 97%, at least about 98%, at least about 99%, at least about 100% homologous or identical to the amino acid sequence set forth in SEQ ID NO: 94.

A5. In certain non-limiting embodiments, the presently disclosed subject matter provides an anti-CD40 antibody or an antigen-binding fragment thereof, comprising a heavy chain variable region comprising the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93.

A6. In certain non-limiting embodiments, the presently disclosed subject matter provides an anti-CD40 antibody or an antigen-binding fragment thereof, comprising a light chain variable region comprising the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

A7. In certain non-limiting embodiments, the presently disclosed subject matter provides an anti-CD40 antibody or an antigen-binding fragment thereof, comprising
(a) a heavy chain variable region comprising the amino acid sequence set forth in SEQ ID NO: 14, SEQ ID NO: 24, SEQ ID NO: 34, SEQ ID NO: 41, SEQ ID NO: 49, SEQ ID NO: 59, SEQ ID NO: 68, SEQ ID NO: 78, SEQ ID NO: 83, or SEQ ID NO: 93; or
(b) a light chain variable region comprising the amino acid sequence set forth in SEQ ID NO: 15, SEQ ID NO: 25, SEQ ID NO: 35, SEQ ID NO: 50, SEQ ID NO: 60, SEQ ID NO: 69, SEQ ID NO: 79, SEQ ID NO: 84, or SEQ ID NO: 94.

A8. The foregoing antibody or antigen-binding fragment thereof of any one of A1-A7, wherein
(a) the heavy chain variable region comprises the amino acid sequence set forth in SEQ ID NO: 14, and the light chain variable region comprises the amino acid sequence set forth in SEQ ID NO: 15;
(b) the heavy chain variable region comprises the amino acid sequence set forth in SEQ ID NO: 24, and the light chain variable region comprises the amino acid sequence set forth in SEQ ID NO: 25;
(c) the heavy chain variable region comprises the amino acid sequence set forth in SEQ ID NO: 34, and the light chain variable region comprises the amino acid sequence set forth in SEQ ID NO: 35;
(d) the heavy chain variable region comprises the amino acid sequence set forth in SEQ ID NO: 41, and the light chain variable region comprises the amino acid sequence set forth in SEQ ID NO: 35;
(e) the heavy chain variable region comprises the amino acid sequence set forth in SEQ ID NO: 49, and the light chain variable region comprises the amino acid sequence set forth in SEQ ID NO: 50;
(f) the heavy chain variable region comprises the amino acid sequence set forth in SEQ ID NO: 59, and the light chain variable region comprises the amino acid sequence set forth in SEQ ID NO: 60;
(g) the heavy chain variable region comprises the amino acid sequence set forth in SEQ ID NO: 68, and the light chain variable region comprises the amino acid sequence set forth in SEQ ID NO: 69;
(h) the heavy chain variable region comprises the amino acid sequence set forth in SEQ ID NO: 78, and the light chain variable region comprises the amino acid sequence set forth in SEQ ID NO: 79;
(i) the heavy chain variable region comprises the amino acid sequence set forth in SEQ ID NO: 83, and the light chain variable region comprises the amino acid sequence set forth in SEQ ID NO: 84; or
(j) the heavy chain variable region comprises the amino acid sequence set forth in SEQ ID NO: 93, and the light chain variable region comprises the amino acid sequence set forth in SEQ ID NO: 94.

A9. The foregoing anti-CD40 antibody or an antigen-binding fragment thereof of A8, wherein:
(a) the heavy chain variable region comprises the amino acid sequence set forth in SEQ ID NO: 14, and the light chain variable region comprises the amino acid sequence set forth in SEQ ID NO: 15; or
(b) the heavy chain variable region comprises the amino acid sequence set forth in SEQ ID NO: 24, and the light chain variable region comprises the amino acid sequence set forth in SEQ ID NO: 25.

A10. In certain non-limiting embodiments, the presently disclosed subject matter provides an anti-CD40 antibody or an antigen-binding fragment thereof, comprising a heavy chain variable region that comprises CDR1, CDR2, and CDR3 domains; and a light chain variable region that comprises CDR1, CDR2, and CDR3 domains, wherein the heavy chain variable region and light chain variable region CDR3 domains are selected from the group consisting of:
(a) a heavy chain variable region CDR3 comprising the amino acid sequence set forth in SEQ ID NO: 10 or a conservative modification thereof; and a light chain variable region CDR3 comprising the amino acid sequence set forth in SEQ ID NO: 13 or a conservative modification thereof;
(b) a heavy chain variable region CDR3 comprising the amino acid sequence set forth in SEQ ID NO: 20 or a conservative modification thereof; and a light chain variable region CDR3 comprising the amino acid sequence set forth in SEQ ID NO: 23 or a conservative modification thereof;
(c) a heavy chain variable region CDR3 comprising the amino acid sequence set forth in SEQ ID NO: 30 or a conservative modification thereof; and a light chain variable region CDR3 comprising the amino acid sequence set forth in SEQ ID NO: 33 or a conservative modification thereof;
(d) a heavy chain variable region CDR3 comprising the amino acid sequence set forth in SEQ ID NO: 40 or a conservative modification thereof; and a light chain variable region CDR3 comprising the amino acid sequence set forth in SEQ ID NO: 33 or a conservative modification thereof;
(e) a heavy chain variable region CDR3 comprising the amino acid sequence set forth in SEQ ID NO: 45 or a conservative modification thereof; and a light chain variable region CDR3 comprising the amino acid sequence set forth in SEQ ID NO: 48 or a conservative modification thereof;
(f) a heavy chain variable region CDR3 comprising the amino acid sequence set forth in SEQ ID NO: 55 or a conservative modification thereof; and a light chain variable region CDR3 comprising the amino acid sequence set forth in SEQ ID NO: 58 or a conservative modification thereof;
(g) a heavy chain variable region CDR3 comprising the amino acid sequence set forth in SEQ ID NO: 65 or a conservative modification thereof; and a light chain variable region CDR3 comprising the amino acid sequence set forth in SEQ ID NO: 67 or a conservative modification thereof;
(h) a heavy chain variable region CDR3 comprising the amino acid sequence set forth in SEQ ID NO: 74 or a conservative modification thereof; and a light chain variable region CDR3 comprising the amino acid sequence set forth in SEQ ID NO: 77 or a conservative modification thereof;
(i) a heavy chain variable region CDR3 comprising the amino acid sequence set forth in SEQ ID NO: 82 or a conservative modification thereof; and a light chain variable region CDR3 comprising the amino acid sequence set forth in SEQ ID NO: 67 or a conservative modification thereof; or
(j) a heavy chain variable region CDR3 comprising the amino acid sequence set forth in SEQ ID NO: 89 or a conservative modification thereof; and a light chain variable region CDR3 comprising the amino acid sequence set forth in SEQ ID NO: 92 or a conservative modification thereof.

A11. The foregoing antibody or antigen-binding fragment thereof of A10, wherein the heavy chain variable region and light chain variable region CDR2 domains are selected from the group consisting of:
(a) a heavy chain variable region CDR2 comprising the amino acid sequence set forth in SEQ ID NO: 9 or a conservative modification thereof; and a light chain variable region CDR2 comprising the amino acid sequence set forth in SEQ ID NO: 12 or a conservative modification thereof;
(b) a heavy chain variable region CDR2 comprising the amino acid sequence set forth in SEQ ID NO: 19 or a conservative modification thereof; and a light chain variable region CDR2 comprising the amino acid sequence set forth in SEQ ID NO: 22 or a conservative modification thereof;
(c) a heavy chain variable region CDR2 comprising the amino acid sequence set forth in SEQ ID NO: 29 or a conservative modification thereof; and a light chain variable region CDR2 comprising the amino acid sequence set forth in SEQ ID NO: 32 or a conservative modification thereof;
(d) a heavy chain variable region CDR2 comprising the amino acid sequence set forth in SEQ ID NO: 39 or a conservative modification thereof; and a light chain variable region CDR2 comprising the amino acid sequence set forth in SEQ ID NO: 32 or a conservative modification thereof;
(e) a heavy chain variable region CDR2 comprising the amino acid sequence set forth in SEQ ID NO: 44 or a conservative modification thereof; and a light chain variable region CDR2 comprising the amino acid sequence set forth in SEQ ID NO: 47 or a conservative modification thereof;
(f) a heavy chain variable region CDR2 comprising the amino acid sequence set forth in SEQ ID NO: 54 or a conservative modification thereof; and a light chain variable region CDR2 comprising the amino acid sequence set forth in SEQ ID NO: 57 or a conservative modification thereof;
(g) a heavy chain variable region CDR2 comprising the amino acid sequence set forth in SEQ ID NO: 64 or a conservative modification thereof; and a light chain variable region CDR2 comprising the amino acid sequence set forth in SEQ ID NO: 22 or a conservative modification thereof;
(h) a heavy chain variable region CDR2 comprising the amino acid sequence set forth in SEQ ID NO: 73 or a conservative modification thereof; and a light chain variable region CDR2 comprising the amino acid sequence set forth in SEQ ID NO: 76 or a conservative modification thereof;
(i) a heavy chain variable region CDR2 comprising the amino acid sequence set forth in SEQ ID NO: 64 or a conservative modification thereof; and a light chain variable region CDR2 comprising the amino acid sequence set forth in SEQ ID NO: 22 or a conservative modification thereof; and
(j) a heavy chain variable region CDR2 comprising the amino acid sequence set forth in SEQ ID NO: 88 or a conservative modification thereof; and a light chain variable region CDR2 comprising the amino acid sequence set forth in SEQ ID NO: 91 or a conservative modification thereof.

A12. The foregoing antibody or antigen-binding fragment thereof of A10 or All, wherein the heavy chain variable region and light chain variable region CDR1 domains are selected from the group consisting of:
(a) a heavy chain variable region CDR1 comprising the amino acid sequence set forth in SEQ ID NO: 8 or a conservative modification thereof; and a light chain variable region CDR1 comprising the amino acid sequence set forth in SEQ ID NO: 11 or a conservative modification thereof;
(b) a heavy chain variable region CDR1 comprising the amino acid sequence set forth in SEQ ID NO: 18 or a conservative modification thereof; and a light chain variable region CDR1 comprising the amino acid sequence set forth in SEQ ID NO: 21 or a conservative modification thereof;
(c) a heavy chain variable region CDR1 comprising the amino acid sequence set forth in SEQ ID NO: 28 or a conservative modification thereof; and a light chain variable region CDR1 comprising the amino acid sequence set forth in SEQ ID NO: 31 or a conservative modification thereof;
(d) a heavy chain variable region CDR1 comprising the amino acid sequence set forth in SEQ ID NO: 38 or a conservative modification thereof; and a light chain variable region CDR1 comprising the amino acid sequence set forth in SEQ ID NO: 31 or a conservative modification thereof;
(e) a heavy chain variable region CDR1 comprising the amino acid sequence set forth in SEQ ID NO: 43 or a conservative modification thereof; and a light chain variable region CDR1 comprising the amino acid sequence set forth in SEQ ID NO: 46 or a conservative modification thereof;
(f) a heavy chain variable region CDR1 comprising the amino acid sequence set forth in SEQ ID NO: 53 or a conservative modification thereof; and a light chain variable region CDR1 comprising the amino acid sequence set forth in SEQ ID NO: 56 or a conservative modification thereof;
(g) a heavy chain variable region CDR1 comprising the amino acid sequence set forth in SEQ ID NO: 63 or a conservative modification thereof; and a light chain variable region CDR1 comprising the amino acid sequence set forth in SEQ ID NO: 66 or a conservative modification thereof;
(h) a heavy chain variable region CDR1 comprising the amino acid sequence set forth in SEQ ID NO: 71 72 or a conservative modification thereof; and a light chain variable region CDR1 comprising the amino acid sequence set forth in SEQ ID NO: 75 or a conservative modification thereof;
(i) a heavy chain variable region CDR1 comprising the amino acid sequence set forth in SEQ ID NO: 63 or a conservative modification thereof; and a light chain variable region CDR1 comprising the amino acid sequence set forth in SEQ ID NO: 66 or a conservative modification thereof; or
(j) a heavy chain variable region CDR1 comprising the amino acid sequence set forth in SEQ ID NO: 87 or a conservative modification thereof; and a light chain variable region CDR1 comprising the amino acid sequence set forth in SEQ ID NO: 90 or a conservative modification thereof.

A13. The foregoing antibody or antigen-binding fragment thereof of any one of A10-A12, wherein one or more of the CDR sequences have up to about 5 amino acid substitutions.

A14. The foregoing antibody or antigen-binding fragment thereof of any one of A10-A13, wherein one or more of the CDR sequences have up to about 3 amino acid substitutions.

A15. In certain non-limiting embodiments, the presently disclosed subject matter provides an anti-CD40 antibody or an antigen-binding fragment thereof, comprising:
(a) a heavy chain variable region comprising a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10;
(b) a heavy chain variable region comprising a CDR1 comprising the amino acid sequence set forth in SEQ ID NO: 18, a CDR2 comprising the amino acid sequence set forth in SEQ ID NO: 19, and a CDR3 comprising the amino acid sequence set forth in SEQ ID NO: 20;
(c) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30;
(d) a heavy chain variable region comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40;
(e) a heavy chain variable region comprising a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45;
(f) a heavy chain variable region comprising a CDR1 comprising the amino acid sequence set forth in SEQ ID NO: 53, a CDR2 comprising the amino acid sequence set forth in SEQ ID NO: 54, and a CDR3 comprising the amino acid sequence set forth in SEQ ID NO: 55;
(g) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65;
(h) a heavy chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74;
(i) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 82; or
(j) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89.

A16. In certain non-limiting embodiments, the presently disclosed subject matter provides an anti-CD40 antibody or an antigen-binding fragment thereof, comprising:
(a) a light chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
(b) a light chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23;
(c) a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(d) a light chain variable region comprising a CDR1 comprising the amino acid sequence set forth in SEQ ID NO: 46, a CDR2 comprising the amino acid sequence set forth in SEQ ID NO: 47, a CDR3 comprising the amino acid sequence set forth in SEQ ID NO: 48;
(e) a light chain variable region comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58;
(f) a light chain variable region comprising a CDR1 comprising the amino acid sequence set forth in SEQ ID NO: 66, a CDR2 comprising the amino acid sequence set forth in SEQ ID NO: 22, a CDR3 comprising the amino acid sequence set forth in SEQ ID NO: 67;
(g) a light chain variable region comprising a CDR1 comprising the amino acid sequence set forth in SEQ ID NO: 75, a CDR2 comprising the amino acid sequence set forth in SEQ ID NO: 76, and a CDR3 comprising the amino acid sequence set forth in SEQ ID NO: 77; or
(h) a light chain variable region comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92.

A17. In certain non-limiting embodiments, the presently disclosed subject matter provides an anti-CD40 antibody or an antigen-binding fragment thereof, comprising:
(a) a heavy chain variable region comprising a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and a light chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
(b) a heavy chain variable region comprising a CDR1 comprising the amino acid sequence set forth in SEQ ID NO: 18, a CDR2 comprising an amino acid sequence set forth in SEQ ID NO: 19, and a CDR3 comprising the amino acid sequence set forth in SEQ ID NO: 20; and a light chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23;
(c) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(d) a heavy chain variable region comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(e) a heavy chain variable region comprising a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and a light chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
(f) a heavy chain variable region comprising a CDR1 comprising the amino acid sequence set forth in SEQ ID NO: 53, a CDR2 comprising the amino acid sequence set forth in SEQ ID NO: 54, and a CDR3 comprising the amino acid sequence set forth in SEQ ID NO: 55; and a light chain variable region comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58;
(g) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65; and a light chain variable region comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67;
(h) a heavy chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and a light chain variable region comprising a CDR1 comprising the amino acid sequence set forth in SEQ ID NO: 75, a CDR2 comprising the amino acid sequence set forth in SEQ ID NO: 76, and a CDR3 comprising the amino acid sequence set forth in SEQ ID NO: 77;
(i) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 82; and a light chain variable region comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67; or
(j) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92.

A18. The foregoing antibody or antigen-binding fragment thereof of A17, wherein:
(a) the heavy chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and the light chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; or
(b) the heavy chain variable region comprises a CDR1 comprising the amino acid sequence set forth in SEQ ID NO: 18, a CDR2 comprising an amino acid sequence set forth in SEQ ID NO: 19, and a CDR3 comprising the amino acid sequence set forth in SEQ ID NO: 20; and the light chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23.

A19. The foregoing antibody or antigen-binding fragment thereof of any one of A1-A18, wherein the antibody or antigen-binding fragment thereof binds to a CD40 comprising the amino acid sequence set forth in SEQ ID NO: 7 or a fragment thereof.

A20. The foregoing antibody or antigen-binding fragment thereof of any one of A1-A19, wherein the antibody comprises a human variable region framework region.

A21. The foregoing antibody or antigen-binding fragment thereof of any one of A1-A20, which is a fully human or an antigen-binding fragment thereof.

A22. The foregoing antibody or antigen-binding fragment thereof of any one of A1-A20, which is a chimeric antibody or an antigen-binding fragment thereof.

A23. The foregoing antibody or antigen-binding fragment thereof of any one of A1-A20, which is a humanized antibody or an antigen-binding fragment thereof.

A24. The foregoing antibody or antigen-binding fragment thereof of any one of A1-A20, which is a murine antibody or an antigen-binding fragment thereof.

A25. The foregoing antibody or antigen-binding fragment thereof of any one of A1-A24, wherein the antigen-binding fragment is a Fab, Fab′, F(ab′)2, variable fragment (Fv), or single chain variable region (scFv).

A26. The foregoing antibody or antigen-binding fragment thereof of A25, wherein the antigen-binding fragment is an scFv.

A27. In certain non-limiting embodiments, the presently disclosed subject matter provides an antibody or an antigen biding fragment thereof, which cross-competes for binding to CD40 with an antibody or an antigen-binding fragment thereof of any one of A1-A26.

A28. In certain non-limiting embodiments, the presently disclosed subject matter provides an antibody or an antigen biding fragment thereof, which binds to the same epitope region on CD40 with an antibody or an antigen-binding fragment thereof of any one of A1-A26.

B1. In certain non-limiting embodiments, the presently disclosed subject matter provides a composition comprising the antibody or antigen-binding fragment thereof of any one of A1-A28.

B2. The foregoing composition of B1, which is a pharmaceutical composition that further comprises a pharmaceutically acceptable carrier.

C1. In certain non-limiting embodiments, the presently disclosed subject matter provides an immunoconjugate comprising the antibody or antigen-binding fragment thereof of any one of A1-A28, linked to a therapeutic agent.

C2. The foregoing immunoconjugate of C1, wherein the therapeutic agent is a drug or a radioactive isotope.

D1. In certain non-limiting embodiments, the presently disclosed subject matter provides a composition comprising the immunoconjugate of C1 or C2.

D2. The foregoing composition of D1, which is a pharmaceutical composition that further comprises a pharmaceutically acceptable carrier.

E1. In certain non-limiting embodiments, the presently disclosed subject matter provides a multi-specific molecule comprising the antibody or antigen-binding fragment thereof of any one of A1-A28, linked to one or more functional moieties.

E2. The foregoing multi-specific molecule of E1, wherein the one or more functional moieties have a different binding specificity than the antibody or antigen binding fragment thereof.

F1. In certain non-limiting embodiments, the presently disclosed subject matter provides a composition comprising the multi-specific molecule of E1 or E2.

F2. The foregoing composition of F1, which is a pharmaceutical composition that further comprises a pharmaceutically acceptable carrier.

G1. In certain non-limiting embodiments, the presently disclosed subject matter provides an Fc fusion protein comprising the antibody or antigen-binding fragment thereof of any one of A1-A28.

G2. The foregoing Fc fusion protein of G1, wherein the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 122 or SEQ ID NO: 124.

G3. The foregoing Fc fusion protein of G1 or G2, wherein the Fc fusion protein comprises the amino acid sequence set forth in SEQ ID NO: 122 or SEQ ID NO: 124.

H1. In certain non-limiting embodiments, the presently disclosed subject matter provides a composition comprising the Fc fusion protein of any one of G1-G3.

H2. The foregoing composition of H1, which is a pharmaceutical composition that further comprises a pharmaceutically acceptable carrier.

I1. In certain non-limiting embodiments, the presently disclosed subject matter provides a nucleic acid that encodes an antibody or antigen-binding fragment thereof of any one of A1-A28.

I2. In certain non-limiting embodiments, the presently disclosed subject matter provides a nucleic acid that encodes a heavy chain variable region of an antibody or antigen-binding fragment thereof of any one of A1-A28.

I3. The foregoing nucleic acid of I2, comprising a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 16, SEQ ID NO: 26, SEQ ID NO: 36, SEQ ID NO: 42, SEQ ID NO: 51, SEQ ID NO: 61, SEQ ID NO: 70, SEQ ID NO: 80, SEQ ID NO: 85, or SEQ ID NO: 95, SEQ ID NO: 118, or SEQ ID NO: 120.

I4. The foregoing nucleic acid of I2 or I3, comprising the nucleotide sequence set forth in SEQ ID NO: 16, SEQ ID NO: 26, SEQ ID NO: 36, SEQ ID NO: 42, SEQ ID NO: 51, SEQ ID NO: 61, SEQ ID NO: 70, SEQ ID NO: 80, SEQ ID NO: 85, or SEQ ID NO: 95, SEQ ID NO: 118, or SEQ ID NO: 120.

I5. In certain non-limiting embodiments, the presently disclosed subject matter provides a nucleic acid that encodes a light chain variable region of an antibody or antigen-binding fragment thereof of any one of A1-A28.

I6. The foregoing nucleic acid of 15, comprising a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 17, SEQ ID NO: 27, SEQ ID NO: 37, SEQ ID NO: 52, SEQ ID NO: 62, SEQ ID NO: 71, SEQ ID NO: 81, SEQ ID NO: 86, or SEQ ID NO: 96, SEQ ID NO: 119, or SEQ ID NO: 121.

I7. The foregoing nucleic acid of I5 or I6, comprising the nucleotide sequence set forth in SEQ ID NO: 17, SEQ ID NO: 27, SEQ ID NO: 37, SEQ ID NO: 52, SEQ ID NO: 62, SEQ ID NO: 71, SEQ ID NO: 81, SEQ ID NO: 86, or SEQ ID NO: 96, SEQ ID NO: 119, or SEQ ID NO: 121.

I8. In certain non-limiting embodiments, the presently disclosed subject matter provides a nucleic acid that encodes an Fc fusion protein of any one of G1-G3.

I9. The foregoing nucleic acid of 18, comprising a nucleotide sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98% or about 99% homologous or identical to the nucleotide sequence set forth in SEQ ID NO: 123 or SEQ ID NO: 125.

I10. The foregoing nucleic acid of I8 or I9, comprising the nucleotide sequence set forth in SEQ ID NO: 123 or SEQ ID NO: 125.

J1. In certain non-limiting embodiments, the presently disclosed subject matter provides a vector comprising the nucleic acid of any one of I1-I10.

K1. In certain non-limiting embodiments, the presently disclosed subject matter provides a host cell comprising the vector of J1.

K2. In certain non-limiting embodiments, the presently disclosed subject matter provides an immunoresponsive cell comprising the antibody or antigen-binding fragment thereof of any one of A1-A28, the immunoconjugate of C1 or C2, the multi-specific molecule of E1 or E2, the Fc fusion protein of any one of G1-G3, the nucleic acid of any one of I1-I10, or the vector of J1.

K3. The foregoing immunoresponsive cell of K2, further comprising antigen-recognizing receptor.

K4. The foregoing immunoresponsive cell of K3, wherein the antigen-recognizing receptor is a recombinant T cell receptor (TCR), a chimeric antigen receptor (CAR), or a TCR like fusion molecule.

K5. The foregoing immunoresponsive cell of K3 or K4, wherein the antigen-recognizing receptor is a CAR.

K6. The foregoing immunoresponsive cell of any one of K2-K5, wherein the antigen is a tumor antigen or a pathogen antigen.

K7. The foregoing immunoresponsive cell of any one of K2-K6, wherein the antigen is a tumor antigen.

K8. The foregoing immunoresponsive cell of K7, wherein the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, ERBB, Tyrosinase related protein 1 (TYRP1, gp75), and Delta-like 3 (DLL3).

K9. The foregoing immunoresponsive cell of any one of K2-K8, wherein the immunoresponsive cell is a cell of the lymphoid lineage or a cell of the myeloid lineage.

K10. The foregoing immunoresponsive cell of any one of K2-K9, wherein the cell is selected from the group consisting of a T cell, a Natural Killer (NK) cell, a B cell, a monocyte, and a macrophage, a pluripotent stem cell from which a lymphoid cell may be differentiated, a pluripotent stem cell from which a myeloid cell may be differentiated, and combinations thereof.

K11. The foregoing immunoresponsive cell of any one of K2-K10, wherein the cell is a T cell.

K12. The foregoing immunoresponsive cell of any one of K2-K10, wherein the cell is a Natural Killer (NK) cell.

K13. The foregoing immunoresponsive cell of any one of K2-K10, wherein the cell is autologous.

K14. The foregoing immunoresponsive cell of any one of K2-K10, wherein the cell is allogeneic.

L1. In certain non-limiting embodiments, the presently disclosed subject matter provides a composition comprising the immunoresponsive cell of any one of K2-K14.

L2. The foregoing composition of L1, which is a pharmaceutical composition further comprising a pharmaceutically acceptable excipient.

M1. In certain non-limiting embodiments, the presently disclosed subject matter provides a method for detecting CD40 in a whole cell, a tissue, or a blood sample, comprising:
(a) contacting a cell, tissue or blood sample with the antibody or antigen-binding fragment thereof of any one of A1-A28, wherein the antibody or antigen-binding fragment thereof comprises a detectable label; and
(b) determining the amount of the labeled antibody or antigen-binding fragment thereof bound to the cell, tissue, or blood sample by measuring the amount of detectable label associated with said cell or tissue, wherein the amount of bound antibody or antigen-binding fragment thereof indicates the amount of CD40 in the cell, tissue or blood sample.

M2. In certain non-limiting embodiments, the presently disclosed subject matter provides a method of preventing and/or treating a disease or disorder in a subject, comprising administering to the subject the antibody or antigen-binding fragment thereof of any one of A1-A28, the immunoconjugate of C1 or C2, the multi-specific molecule of E1 or E2, the Fc fusion protein of any one of G1-G3, the immunoresponsive cell of any one of K2-K14, or the composition of any one of B1, B2, D1, F1, F2, H1, and H2.

M3. The foregoing method of M2, wherein the disease or disorder is a tumor.

M4. The foregoing method of M2, wherein the disease or disorder is a pathogen infection.

M5. The foregoing method of M2, wherein the disease or disorder is an autoimmune disease.

M6. The foregoing method of M2, wherein the disease or disorder is an infectious disease.

M7. In certain non-limiting embodiments, the presently disclosed subject matter provides a method of reducing tumor burden in a subject, comprising administering to the subject the antibody or antigen-binding fragment thereof of any one of A1-A28, the immunoconjugate of C1 or C2, the multi-specific molecule of E1 or E2, the Fc fusion protein of any one of G1-G3, the immunoresponsive cell of any one of K2-K14, or the composition of any one of B1, B2, D1, F1, F2, H1, and H2.

M8. The foregoing method of M7, wherein the method reduces the number of the tumor cells, reduces the tumor size, and/or eradicates the tumor in the subject.

M9. In certain non-limiting embodiments, the presently disclosed subject matter provides a method of treating and/or preventing a tumor in a subject, comprising administering to the subject the antibody or antigen-binding fragment thereof of any one of A1-A28, the immunoconjugate of C1 or C2, the multi-specific molecule of E1 or E2, the Fc fusion protein of any one of G1-G3, the immunoresponsive cell of any one of K2-K14, or the composition of any one of B1, B2, D1, F1, F2, H1, and H2.

M10. In certain non-limiting embodiments, the presently disclosed subject matter provides a method of increasing or lengthening survival of a subject having a tumor, comprising administering to the subject the antibody or antigen-binding fragment thereof of any one of A1-A28, the immunoconjugate of C1 or C2, the multi-specific molecule of E1 or E2, the Fc fusion protein of any one of G1-G3, the immunoresponsive cell of any one of K2-K14, or the composition of any one of B1, B2, D1, F1, F2, H1, and H2.

M11. The foregoing method of M10, wherein the method reduces or eradicates tumor burden in the subject.

M12. The foregoing method of any one of M3, and M7-M11, wherein the tumor is cancer.

M13. The foregoing method of any one of M3, and M7-M12, wherein the tumor is hematological cancer or solid tissue cancer.

M14. The foregoing method of M13, wherein the cancer is selected from the group consisting of Hodgkin lymphoma, non-Hodgkin's lymphoma, B-cell lymphomas, acute myelogenous leukemia, chronic myelogenous leukemia, acute lymphoblastic leukemia, chronic lymphocytic leukemia, hairy cell leukemia, multiple myeloma, melanoma, small cell lung cancer, sarcoma, glioma, neuroblastoma, prostate cancer, breast cancer, lung cancer, pancreatic cancer, gastric cancer, esophageal cancer, colon cancer, rectal cancer, bladder cancer, renal cell carcinoma, hepatocellular carcinoma, cholangiocarcinoma, Merkel cell carcinoma, head and neck cancer, thyroid cancer, salivary gland cancer, ovarian cancer, endometrial cancer, cervical cancer, germ cell tumor, testicular cancer, basal cell carcinoma, squamous cell carcinoma, mesothelioma, retinoblastoma, neurofibroma, pheochromocytoma, and adrenal gland tumor.

M15. The foregoing method of any one of M2-M14, wherein the subject is a human.

N1. A kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of any one of A1-A28, the immunoconjugate of C1 or C2, the multi-specific molecule of E1 or E2, the Fc fusion protein of any one of G1-G3, the immunoresponsive cell of any one of K2-K14, or the composition of any one of B1, B2, D1, F1, F2, H1, and H2.

N2. The foregoing kit of N1, wherein the kit further comprises written instructions for using the antibody or antigen-binding fragment thereof, immunoconjugate, multi-specific molecule, immunoresponsive cell, or composition for treating or ameliorating a disease or disorder in a subject, treating or ameliorating a disease or disorder in a subject, reducing tumor burden in a subject, treating and/or preventing a tumor in a subject, and/or increasing or lengthening survival of a subject having a tumor.

### 6. EXAMPLES

The following examples are put forth so as to provide those of ordinary skill in the art with a complete disclosure and description of how to make and use the antibodies, multi-specific antibodies, compositions comprising thereof, screening, and therapeutic methods of the presently disclosed subject matter, and are not intended to limit the scope of what the inventors regard as their presently disclosed subject matter. It is understood that various other embodiments may be practiced, given the general description provided above.

### Example 1—Generation of the Presently Disclosed Anti-CD40 Antibodies and scFvs

Stimulation of the CD40 pathway in APCs has been recognized as a highly effective way to activate APCs. CD40 agonists have demonstrated antitumor activity as monotherapy in human cancers. Currently, there is great interest in the use of CD40 stimulation in the treatment of cancer, in part because of compelling early-phase data incorporating the CD40 agonist mAb APX005M (Apexigen) along with chemotherapy and PD-1 blockade in the first-line treatment of metastatic pancreatic cancer. Several approaches to induce CD40 signaling with an agonist agent have been explored. For example, certain CD40 agonist antibodies with a modified Fc portion that increases binding to FcγRIIB, which is present on intra-tumoral myeloid cells, can more potently stimulate APCs. This has led to the production of a series of CD40 agonist antibodies designed to have increased FcγRIIB binding, including APX005M14 or 2141-V1115.

Instead of relying on Fc receptor engagement, the presently disclosed subject matter focused on identifying CD40 agonist antibodies with certain other intrinsic features, including competition with the binding site of CD40L, the ligand for CD40. Binding CD40 at the natural ligand binding site can increase agonist activity, and can further reduce inter-patient variability by preventing uncontrolled activation of CD40 by endogenous CD40L.

Antibody generation was carried out by immunizing mice with recombinant proteins either sourced commercially or produced in-house. Hybridoma were generated by electrofusion, and the supernatants were screened by enzyme-linked immunosorbent assay (ELISA) on recombinant proteins. The primary hybridoma screen by ELISA using soluble, His-tagged human CD40 (full-length and IgC2) resulted in more than 500 hits. Upon completion of the binding and cross-reactivity experiments delineated in the screening funnel, 27 hybridoma were selected for subcloning. These included antibodies with strong agonist activity without crosslinking (e.g., signal does not change upon crosslinking or signal goes down upon crosslinking) or antibodies with increased agonism with crosslinking.

Finally, up to two clonal hybridomas (designated as “Clone “A″” and/or “Clone “B″”, respectively) were generated and cryopreserved.

Next, the hybridomas were selected for subcloning to identify monoclonal antibodies against CD40. Briefly, cells were plated at a 1 cell per well density in 384-well plates, confirmed by microscopy, and expanded. After expansion, supernatants were screened by ELISA against hCD40. The mAbs were detected using anti-mouse IgG-conjugated to HRP.

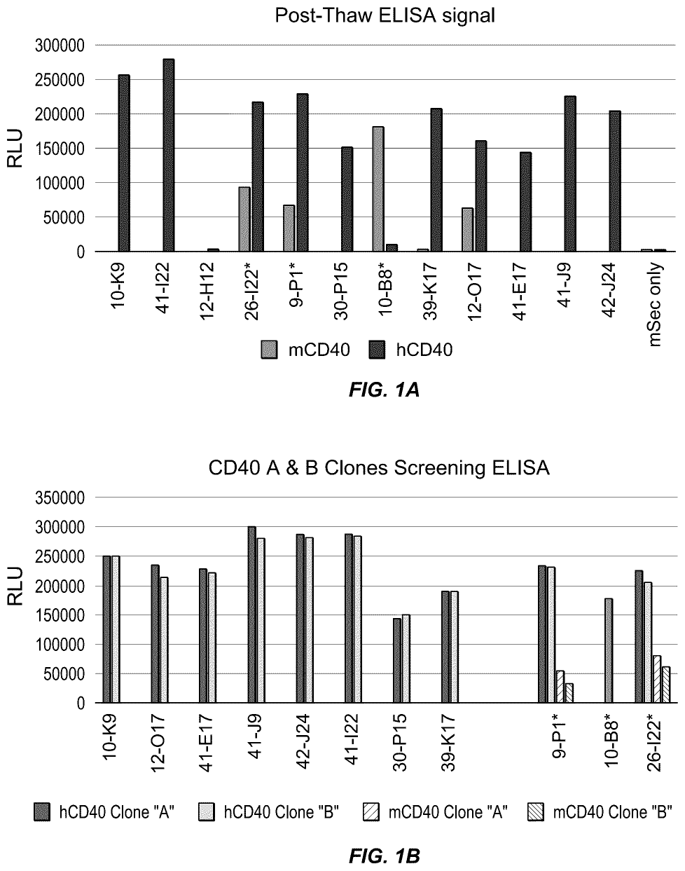

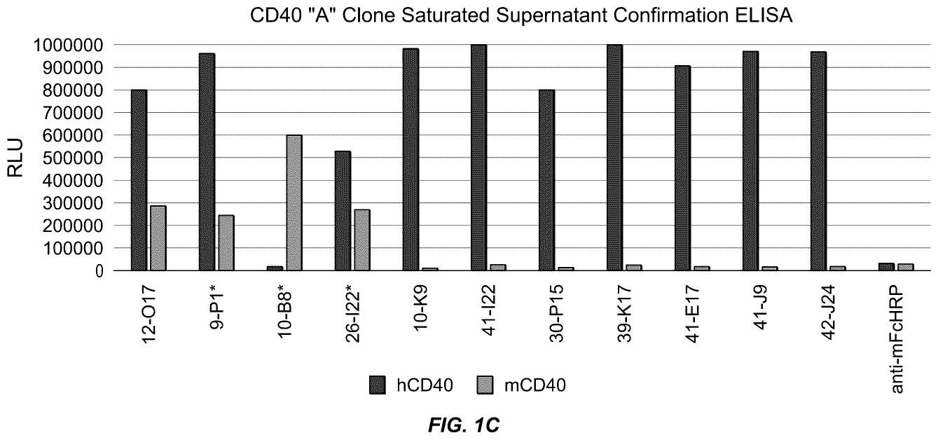

As seen in FIGS. 1A and 1B, all hybridomas detected human CD40 and three hybridomas (e.g., 9-P1, 10-B8, and 26-I22) also detected mouse CD40. Additional confirmation data are shown in FIG. 1C. Thus, 10 unique hybridomas were successfully cloned.

Finally, antibodies were sequenced, and CDR analysis was performed using sequencing data (CDR regions were defined using VBASE2, www.vbase2.org/).

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An anti-CD40 antibody or an antigen-binding fragment thereof, comprising:
(a) a heavy chain variable region comprising a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and a light chain variable region comprising a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13;
(b) a heavy chain variable region comprising a CDR1 comprising the amino acid sequence set forth in SEQ ID NO: 18, a CDR2 comprising an amino acid sequence set forth in SEQ ID NO: 19, and a CDR3 comprising the amino acid sequence set forth in SEQ ID NO: 20; and a light chain variable region comprising a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23;
(c) a heavy chain variable region comprising a CDR1 comprising the amino acid sequence set forth in SEQ ID NO: 28, a CDR2 comprising the amino acid sequence set forth in SEQ ID NO: 29, and a CDR3 comprising the amino acid sequence set forth in SEQ ID NO: 30; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(d) a heavy chain variable region comprising a CDR1 comprising the amino acid sequence set forth in SEQ ID NO: 38, a CDR2 comprising the amino acid sequence set forth in SEQ ID NO: 39, and a CDR3 comprising the amino acid sequence set forth in SEQ ID NO: 40; and a light chain variable region comprising a CDR1 comprising the amino acid sequence set forth in SEQ ID NO: 31, a CDR2 comprising the amino acid sequence set forth in SEQ ID NO: 32, and a CDR3 comprising the amino acid sequence set forth in SEQ ID NO: 33;
(e) a heavy chain variable region comprising a CDR1 comprising the amino acid sequence set forth in SEQ ID NO: 43, a CDR2 comprising the amino acid sequence set forth in SEQ ID NO: 44, and a CDR3 comprising the amino acid sequence set forth in SEQ ID NO: 45; and a light chain variable region comprising a CDR1 comprising the amino acid sequence set forth in SEQ ID NO: 46, a CDR2 comprising the amino acid sequence set forth in SEQ ID NO: 47, and a CDR3 comprising the amino acid sequence set forth in SEQ ID NO: 48;
(f) a heavy chain variable region comprising a CDR1 comprising the amino acid sequence set forth in SEQ ID NO: 53, a CDR2 comprising the amino acid sequence set forth in SEQ ID NO: 54, and a CDR3 comprising the amino acid sequence set forth in SEQ ID NO: 55; and a light chain variable region comprising a CDR1 comprising the amino acid sequence set forth in SEQ ID NO: 56, a CDR2 comprising the amino acid sequence set forth in SEQ ID NO: 57, and a CDR3 comprising the amino acid sequence set forth in SEQ ID NO: 58;
(g) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 65; and a light chain variable region comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67;
(h) a heavy chain variable region comprising a CDR1 comprising the amino acid sequence set forth in SEQ ID NO: 72, a CDR2 comprising the amino acid sequence set forth in SEQ ID NO: 73, and a CDR3 comprising the amino acid sequence set forth in SEQ ID NO: 74; and a light chain variable region comprising a CDR1 comprising the amino acid sequence set forth in SEQ ID NO: 75, a CDR2 comprising the amino acid sequence set forth in SEQ ID NO: 76, and a CDR3 comprising the amino acid sequence set forth in SEQ ID NO: 77;
(i) a heavy chain variable region comprising a CDR1 comprising the amino acid sequence set forth in SEQ ID NO: 63, a CDR2 comprising the amino acid sequence set forth in SEQ ID NO: 64, and a CDR3 comprising the amino acid sequence set forth in SEQ ID NO: 82; and a light chain variable region comprising a CDR1 comprising the amino acid sequence set forth in SEQ ID NO: 66, a CDR2 comprising the amino acid sequence set forth in SEQ ID NO: 22, and a CDR3 comprising the amino acid sequence set forth in SEQ ID NO: 67; or
(j) a heavy chain variable region comprising a CDR1 comprising the amino acid sequence set forth in SEQ ID NO: 87, a CDR2 comprising the amino acid sequence set forth in SEQ ID NO: 88, and a CDR3 comprising the amino acid sequence set forth in SEQ ID NO: 89; and a light chain variable region comprising a CDR1 comprising the amino acid sequence set forth in SEQ ID NO: 90, a CDR2 comprising the amino acid sequence set forth in SEQ ID NO: 91, and a CDR3 comprising the amino acid sequence set forth in SEQ ID NO: 92.

2. The antibody or antigen-binding fragment thereof of claim 1, wherein:
(a) the heavy chain variable region comprises a CDR1 comprising the amino acid sequence set forth in SEQ ID NO: 8, a CDR2 comprising the amino acid sequence set forth in SEQ ID NO: 9, and a CDR3 comprising the amino acid sequence set forth in SEQ ID NO: 10; and the light chain variable region comprises a CDR1 comprising the amino acid sequence set forth in SEQ ID NO: 11, a CDR2 comprising the amino acid sequence set forth in SEQ ID NO: 12, and a CDR3 comprising the amino acid sequence set forth in SEQ ID NO: 13; or
(b) the heavy chain variable region comprises a CDR1 comprising the amino acid sequence set forth in SEQ ID NO: 18, a CDR2 comprising an amino acid sequence set forth in SEQ ID NO: 19, and a CDR3 comprising the amino acid sequence set forth in SEQ ID NO: 20; and the light chain variable region comprises a CDR1 comprising the amino acid sequence set forth in SEQ ID NO: 21, a CDR2 comprising the amino acid sequence set forth in SEQ ID NO: 22, and a CDR3 comprising the amino acid sequence set forth in SEQ ID NO: 23.

3. The antibody or antigen-binding fragment thereof of claim 1, wherein
(a) the heavy chain variable region comprises the amino acid sequence set forth in SEQ ID NO: 14, and the light chain variable region comprises the amino acid sequence set forth in SEQ ID NO: 15;
(b) the heavy chain variable region comprises the amino acid sequence set forth in SEQ ID NO: 24, and the light chain variable region comprises the amino acid sequence set forth in SEQ ID NO: 25;
(c) the heavy chain variable region comprises the amino acid sequence set forth in SEQ ID NO: 34, and the light chain variable region comprises the amino acid sequence set forth in SEQ ID NO: 35;
(d) the heavy chain variable region comprises the amino acid sequence set forth in SEQ ID NO: 41, and the light chain variable region comprises the amino acid sequence set forth in SEQ ID NO: 35;
(e) the heavy chain variable region comprises the amino acid sequence set forth in SEQ ID NO: 49, and the light chain variable region comprises the amino acid sequence set forth in SEQ ID NO: 50;
(f) the heavy chain variable region comprises the amino acid sequence set forth in SEQ ID NO: 59, and the light chain variable region comprises the amino acid sequence set forth in SEQ ID NO: 60;
(g) the heavy chain variable region comprises the amino acid sequence set forth in SEQ ID NO: 68, and the light chain variable region comprises the amino acid sequence set forth in SEQ ID NO: 69;
(h) the heavy chain variable region comprises the amino acid sequence set forth in SEQ ID NO: 78, and the light chain variable region comprises the amino acid sequence set forth in SEQ ID NO: 79;
(i) the heavy chain variable region comprises the amino acid sequence set forth in SEQ ID NO: 83, and the light chain variable region comprises the amino acid sequence set forth in SEQ ID NO: 84; or
(j) the heavy chain variable region comprises the amino acid sequence set forth in SEQ ID NO: 93, and the light chain variable region comprises the amino acid sequence set forth in SEQ ID NO: 94.

4. The anti-CD40 antibody or an antigen-binding fragment thereof of claim 1, wherein:
(a) the heavy chain variable region comprises the amino acid sequence set forth in SEQ ID NO: 14, and the light chain variable region comprises the amino acid sequence set forth in SEQ ID NO: 15; or
(b) the heavy chain variable region comprises the amino acid sequence set forth in SEQ ID NO: 24, and the light chain variable region comprises the amino acid sequence set forth in SEQ ID NO: 25.

5. The antibody or antigen-binding fragment thereof of claim 1, wherein the antigen-binding fragment is an scFv.

6. A pharmaceutical composition comprising the antibody or antigen-binding fragment thereof of any one of claim 1.

7. An immunoconjugate comprising the antibody or antigen-binding fragment thereof of any one of claim 1, linked to a therapeutic agent.

8. The immunoconjugate of claim 7, wherein the therapeutic agent is a drug or a radioactive isotope.

9. A multi-specific molecule comprising the antibody or antigen-binding fragment thereof of claim 1, linked to one or more functional moieties.

10. The multi-specific molecule of claim 9, wherein the one or more functional moieties have a different binding specificity than the antibody or antigen binding fragment thereof.

11. An Fc fusion protein comprising the antibody or antigen-binding fragment thereof of claim 1.

12. The Fc fusion protein of claim 12, wherein the Fc fusion protein comprises an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% identical to the amino acid sequence set forth in SEQ ID NO: 122 or SEQ ID NO: 124.

13. The Fc fusion protein of claim 12, wherein the Fc fusion protein comprises the amino acid sequence set forth in SEQ ID NO: 122 or SEQ ID NO: 124.

14. A pharmaceutical composition comprising the Fc fusion protein of claim 12.

15. A nucleic acid that encodes an antibody or antigen-binding fragment thereof of claim 1.

16. A nucleic acid that encodes an Fc fusion protein of claim 12.

17. A vector comprising the nucleic acid of claim 1.

18. A host cell comprising the vector of claim 17.

19. An immunoresponsive cell comprising the antibody or antigen-binding fragment thereof of claim 1.

20. The immunoresponsive cell of claim 19, further comprising antigen-recognizing receptor selected from a recombinant T cell receptor (TCR), a chimeric antigen receptor (CAR), or a TCR like fusion molecule.

21. The immunoresponsive cell of claim 20, wherein the antigen is a tumor antigen selected from the group consisting of CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, ERBB, Tyrosinase related protein 1 (TYRP1, gp75), and Delta-like 3 (DLL3).

22. The immunoresponsive cell of claim 19, wherein the cell is selected from the group consisting of a T cell, a Natural Killer (NK) cell, a B cell, a monocyte, and a macrophage, a pluripotent stem cell from which a lymphoid cell may be differentiated, a pluripotent stem cell from which a myeloid cell may be differentiated, and combinations thereof.

23. The immunoresponsive cell of claim 19, wherein the cell is autologous or allogeneic.

24. A composition comprising the immunoresponsive cell of claim 19.

25. A method for detecting CD40 in a whole cell, a tissue, or a blood sample, comprising:
(a) contacting a cell, tissue or blood sample with the antibody or antigen-binding fragment thereof of claim 1, wherein the antibody or antigen-binding fragment thereof comprises a detectable label; and
(b) determining the amount of the labeled antibody or antigen-binding fragment thereof bound to the cell, tissue or blood sample by measuring the amount of detectable label associated with said cell or tissue, wherein the amount of bound antibody or antigen-binding fragment thereof indicates the amount of CD40 in the cell, tissue or blood sample.

26. A method of preventing and/or treating a tumor, a pathogen infection, an autoimmune disease, or an infectious disease in a subject, comprising administering to the subject the antibody or antigen-binding fragment thereof of claim 1.

27. A method of reducing tumor burden in a subject, treating and/or preventing a tumor in a subject, and/or increasing or lengthening survival of a subject having a tumor, the method comprising administering to the subject the antibody or antigen-binding fragment thereof of claim 1.

28. The method of claim 27, wherein the tumor is a cancer is selected from the group consisting of Hodgkin lymphoma, non-Hodgkin's lymphoma, B-cell lymphomas, acute myelogenous leukemia, chronic myelogenous leukemia, acute lymphoblastic leukemia, chronic lymphocytic leukemia, hairy cell leukemia, multiple myeloma, melanoma, small cell lung cancer, sarcoma, glioma, neuroblastoma, prostate cancer, breast cancer, lung cancer, pancreatic cancer, gastric cancer, esophageal cancer, colon cancer, rectal cancer, bladder cancer, renal cell carcinoma, hepatocellular carcinoma, cholangiocarcinoma, Merkel cell carcinoma, head and neck cancer, thyroid cancer, salivary gland cancer, ovarian cancer, endometrial cancer, cervical cancer, germ cell tumor, testicular cancer, basal cell carcinoma, squamous cell carcinoma, mesothelioma, retinoblastoma, neurofibroma, pheochromocytoma, and adrenal gland tumor.

29. The method of claim 27, wherein the subject is a human.

30. A kit for treating or ameliorating a disease or disorder in a subject, reducing tumor burden in a subject, treating and/or preventing a tumor in a subject, and/or increasing or lengthening survival of a subject having a tumor, comprising the antibody or antigen-binding fragment thereof of claim 1.


SEQUENCE LISTING (153 sequences)

SEQ ID NO: 1 - protein, 19 aa, synthetic construct
  REGION 1..19: note = Description of sequence: Linker
GGGGSGGGGSGGGSGGGGS

SEQ ID NO: 2 - protein, 15 aa, synthetic construct
  REGION 1..15: note = Description of sequence: Linker
GGGGSGGGGSGGGGS

SEQ ID NO: 3 - protein, 24 aa, synthetic construct
  REGION 1..24: note = Description of sequence: Linker
GGGGSGGGGSGGGGSGGGSGGGGS

SEQ ID NO: 4 - protein, 29 aa, synthetic construct
  REGION 1..29: note = Description of sequence: Linker
GGGGSGGGGSGGGGSGGGGSGGGSGGGGS

SEQ ID NO: 5 - protein, 5 aa, synthetic construct
  REGION 1..5: note = Description of sequence: Linker
GGGGS

SEQ ID NO: 6 - protein, 10 aa, synthetic construct
  REGION 1..10: note = Description of sequence: Linker
GGGGSGGGGS

SEQ ID NO: 7 - protein, 277 aa, Homo sapiens
  REGION 1..277: note = Description of sequence: Human CD40
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 8 - protein, 8 aa, synthetic construct
  REGION 1..8: note = Description of sequence: VH CDR1
GFTFSSYG

SEQ ID NO: 9 - protein, 8 aa, synthetic construct
  REGION 1..8: note = Description of sequence: VH CDR2
ISSDGSHT

SEQ ID NO: 10 - protein, 12 aa, synthetic construct
  REGION 1..12: note = Description of sequence: VH CDR3
VRQGRYVPSFDV

SEQ ID NO: 11 - protein, 6 aa, synthetic construct
  REGION 1..6: note = Description of sequence: VL CDR1
QSIRNN

SEQ ID NO: 12 - skipped

SEQ ID NO: 13 - protein, 9 aa, synthetic construct
  REGION 1..9: note = Description of sequence: VL CDR3
HQTNNWPFT

SEQ ID NO: 14 - protein, 119 aa, synthetic construct
  REGION 1..119: note = Description of sequence: Full VH
EVQLVESGGDLVKPGGSLKLSCAASGFTFSSYGMSWVRQTPDKRLEWVATISSDGSHTYYPDSVKGRFTISRDIAKNTLYLQMSSLKSEDTAMYYCVRQGRYVPSFDVWGAGTTVTVSS

SEQ ID NO: 15 - protein, 107 aa, synthetic construct
  REGION 1..107: note = Description of sequence: Full VL
DIVLTQSPATLSVTPGDSVSLSCRASQSIRNNLHWYQQKSHESPRLLIKDASQSISGTPSRFSGSGSGTDFTLSINSVETEDFGMYFCHQTNNWPFTFGSGTKLEIK

SEQ ID NO: 16 - DNA, 357 bp, synthetic construct
  misc_feature 1..357: note = Description of sequence: DNA for Full VH
  source 1..357: mol_type = other DNA
gaggtgcagctggtggagtctgggggagacttagtgaagcctggagggtccctgaaactctcctgtgcagcctctggattcactttcagtagctatggcatgtcttgggttcgccagactccagacaagaggctggagtgggtcgcaaccattagtagtgatggtagtcacacctactatccagacagtgtgaaggggcgattcaccatctccagagacattgccaagaacaccctgtacctgcaaatgagcagtctgaagtctgaggacacagccatgtattactgtgtaagacaggggaggtacgtcccctccttcgatgtctggggcgcagggaccacggtcaccgtctcctca

SEQ ID NO: 17 - DNA, 321 bp, synthetic construct
  misc_feature 1..321: note = Description of sequence: DNA for Full VL
  source 1..321: mol_type = other DNA
gatattgtgctaactcagtctcccgccaccctgtctgtgactccaggagatagcgtcagtctttcctgcagggccagccaaagtattagaaacaacctacactggtatcaacaaaaatcacatgagtctccaaggcttctcatcaaggatgcttcccagtccatttctgggaccccctccaggttcagtggcagtggctcagggacagatttcactctcagtatcaacagtgtggagactgaagattttggaatgtatttctgtcaccagactaacaattggccattcacgttcggctcggggacaaagttggaaataaaa

SEQ ID NO: 18 - protein, 8 aa, synthetic construct
  REGION 1..8: note = Description of sequence: VH CDR 3
GYTFINYW

SEQ ID NO: 19 - protein, 8 aa, synthetic construct
  REGION 1..8: note = Description of sequence: VH CDR 2
IYPGSSST

SEQ ID NO: 20 - protein, 11 aa, synthetic construct
  REGION 1..11: note = Description of sequence: VH CDR 3
ARSVGYWYFDV

SEQ ID NO: 21 - protein, 12 aa, synthetic construct
  REGION 1..12: note = Description of sequence: VL CDR 1
QSLFFSGNQKNF

SEQ ID NO: 22 - skipped

SEQ ID NO: 23 - protein, 9 aa, synthetic construct
  REGION 1..9: note = Description of sequence: VL CDR 3
QQYYSYPFT

SEQ ID NO: 24 - protein, 118 aa, synthetic construct
  REGION 1..118: note = Description of sequence: Full VH
QVQLQQPGAELVKPGASVKLSCKASGYTFINYWINWMRQRPGQGLEWIGNIYPGSSSTTYNEKFKSKAALTVDTSSSTAYMQLSSLTSDDSAVYFCARSVGYWYFDVWGAGTTVTVSS

SEQ ID NO: 25 - protein, 113 aa, synthetic construct
  REGION 1..113: note = Description of sequence: Full VL
DIVMSQSPSSLAVSVGEKVTLSCKSSQSLFFSGNQKNFLAWYQQKPGQSPNLLIFWASTRESGVPDRFTGSGSGTDFTLTISSVKAEDLAVYYCQQYYSYPFTFGSGTKLEIK

SEQ ID NO: 26 - DNA, 354 bp, synthetic construct
  misc_feature 1..354: note = Description of sequence: DNA for Full VH
  source 1..354: mol_type = other DNA
caggtccaactgcagcagcctggtgctgagcttgtgaagcctggggcctcagtgaagctgtcctgcaaggcttccggctacactttcattaattactggataaattggatgagacagaggcctggacaaggccttgaatggattggaaatatttatcctggtagtagtagtactacatacaatgagaagttcaagagcaaggccgcactgactgtagacacatcctccagcacagcctacatgcagctcagcagcctgacatctgacgactctgcggtctatttttgtgcaagatctgttggctactggtacttcgatgtctggggcgcagggaccacggtcaccgtctcctca

SEQ ID NO: 27 - DNA, 339 bp, synthetic construct
  misc_feature 1..339: note = Description of sequence: DNA for Full VL
  source 1..339: mol_type = other DNA
gacattgtgatgtcacagtctccatcctccctagctgtgtcagttggagagaaggttactctgagctgcaagtccagtcagagccttttttttagtggcaatcaaaagaacttcttggcctggtaccagcagaaaccagggcagtctcctaacctgctgattttctgggcatccactagggaatctggggtccctgatcgcttcacaggcagtggatctgggacagatttcactctcaccatcagcagtgtgaaggctgaagacctggcagtttattactgtcagcaatattatagctatccattcacgttcggctcggggacaaagctggaaataaaa

SEQ ID NO: 28 - protein, 8 aa, synthetic construct
  REGION 1..8: note = Description of sequence: VH CDR1
GYIFTNYW

SEQ ID NO: 29 - protein, 8 aa, synthetic construct
  REGION 1..8: note = Description of sequence: VH CDR2
IHPSDSES

SEQ ID NO: 30 - protein, 13 aa, synthetic construct
  REGION 1..13: note = Description of sequence: VH CDR3
ARSPLTKVVTTDF

SEQ ID NO: 31 - protein, 5 aa, synthetic construct
  REGION 1..5: note = Description of sequence: VL CDR1
SSVNY

SEQ ID NO: 32 - skipped

SEQ ID NO: 33 - protein, 9 aa, synthetic construct
  REGION 1..9: note = Description of sequence: VL CDR3
QQWSSNPLT

SEQ ID NO: 34 - protein, 120 aa, synthetic construct
  REGION 1..120: note = Description of sequence: Full VH
QVQLQQPGAELVKPGASVKLSCTASGYIFTNYWMNWVKQRPGRGLEWIGRIHPSDSESLYNQKFKSKAILTVDKSSSTAYIQLSSLTSEDSAVYYCARSPLTKVVTTDFWGQGTTLTVSS

SEQ ID NO: 35 - protein, 106 aa, synthetic construct
  REGION 1..106: note = Description of sequence: Full VL
EIVLTQSPAIMPASPGEKVTMTCSASSSVNYMHWYQQKSGTSPKRWIYDTSKLASGVPVRFSGSGSGTSYSLTISSMEAEDAATYYCQQWSSNPLTFGGGTKLEIK

SEQ ID NO: 36 - DNA, 360 bp, synthetic construct
  misc_feature 1..360: note = Description of sequence: DNA for Full VH
  source 1..360: mol_type = other DNA
caggtccaattgcagcagcctggggctgaactggtgaagcctggggcttcagtgaagctgtcctgcacggcttctggctacattttcaccaactactggatgaactgggtgaagcagaggcctggacgaggcctcgagtggattggaaggattcatccttctgatagtgaaagtctctacaatcaaaagttcaagagcaaggccatactgactgtagacaaatcctccagcacagcctacatccaactcagcagcctgacatctgaggactctgcggtctattactgtgcaagatcccctcttactaaggttgtaactacggacttctggggccaaggcaccactctcacagtctcctca

SEQ ID NO: 37 - DNA, 318 bp, synthetic construct
  misc_feature 1..318: note = Description of sequence: DNA for Full VL
  source 1..318: mol_type = other DNA
gaaattgttctcacccagtctccagcaatcatgcctgcatctccaggggagaaggtcaccatgacctgcagtgccagctcaagtgtaaattacatgcactggtaccagcagaagtcaggcacctcccccaaaagatggatttatgacacatccaaactggcttctggagtccctgttcgcttcagtggcagtgggtctgggacctcttactctctcacaatcagcagcatggaggctgaagatgctgccacttattactgccagcagtggagtagtaacccactcacgttcggaggggggaccaagctggaaataaaa

SEQ ID NO: 38 - protein, 9 aa, synthetic construct
  REGION 1..9: note = Description of sequence: VH CDR1
GYSITSDYS

SEQ ID NO: 39 - protein, 7 aa, synthetic construct
  REGION 1..7: note = Description of sequence: VH CDR2
IYYSGST

SEQ ID NO: 40 - protein, 12 aa, synthetic construct
  REGION 1..12: note = Description of sequence: VHCDR3
ARFYYGWSYFDY

SEQ ID NO: 41 - protein, 119 aa, synthetic construct
  REGION 1..119: note = Description of sequence: Full VH
DVQLQESGPDLVKPSQSLSLTCTVTGYSITSDYSWHWIRQFPGNKLEWMGYIYYSGSTNYNPSLKSRISITRDTSKNQFFLQLNSVTSEDTATYYCARFYYGWSYFDYWGQGTSLTVSS

SEQ ID NO: 42 - DNA, 357 bp, synthetic construct
  misc_feature 1..357: note = Description of sequence: DNA for Full VH
  source 1..357: mol_type = other DNA
gatgtgcagcttcaggagtcaggacctgacctggtgaaaccttctcagtcactttcactcacctgcactgtcactggctactccatcaccagtgattatagctggcactggatccggcagtttccaggaaacaaactggaatggatgggctacatatattacagtggtagcactaactacaacccatctctcaaaagtcgaatctctatcactcgagacacatccaagaaccagttcttcctgcaactgaattctgtgacttctgaggacacagccacatattactgtgcaagattttactacggttggagttactttgactactggggccaaggcaccagtctcacagtctcctca

SEQ ID NO: 43 - protein, 8 aa, synthetic construct
  REGION 1..8: note = Description of sequence: VH CDR1
GFTFSTFG

SEQ ID NO: 44 - protein, 8 aa, synthetic construct
  REGION 1..8: note = Description of sequence: VH CDR2
ISGGSSTI

SEQ ID NO: 45 - protein, 13 aa, synthetic construct
  REGION 1..13: note = Description of sequence: VH CDR 3
VRPYGSVHYAMDY

SEQ ID NO: 46 - protein, 6 aa, synthetic construct
  REGION 1..6: note = Description of sequence: VL CDR1
QDINNY

SEQ ID NO: 47 - skipped

SEQ ID NO: 48 - protein, 9 aa, synthetic construct
  REGION 1..9: note = Description of sequence: VL CDR3
QQGNTLPWT

SEQ ID NO: 49 - protein, 120 aa, synthetic construct
  REGION 1..120: note = Description of sequence: Full VH
DVQLVESGGGLVQPGGSRKLSCAASGFTFSTFGMHWVRQAPEKGLEWVAYISGGSSTIYYADTVKGRFTISRDNPKNTLFLQMTSLRSEDTAMYYCVRPYGSVHYAMDYWGQGTSVTVSS

SEQ ID NO: 50 - protein, 107 aa, synthetic construct
  REGION 1..107: note = Description of sequence: Full VL
DIQMTQTTSSLSASLGDRVTISCRASQDINNYLNWYQQKPDGTVKLLIYYTSRFHSGVPSRFSGSGSGTDYSLTISNLEQEDIATYFCQQGNTLPWTFGGGTKLDIK

SEQ ID NO: 51 - DNA, 360 bp, synthetic construct
  misc_feature 1..360: note = Description of sequence: DNA for Full VH
  source 1..360: mol_type = other DNA
gatgtgcagctggtggagtctgggggaggcttagtgcagcctggagggtcccggaaactctcctgtgcagcctctggattcactttcagtacctttggaatgcactgggttcgtcaggctccagagaaggggctggagtgggtcgcatacattagtggtggcagtagtaccatctactatgcagacacagtgaagggccgattcaccatctccagagacaatcccaagaacaccctgttcctgcaaatgaccagtctaaggtctgaggacacggccatgtattactgtgtaagaccctacggtagtgttcactatgctatggactactggggtcaaggaacctcagtcaccgtctcctca

SEQ ID NO: 52 - DNA, 321 bp, synthetic construct
  misc_feature 1..321: note = Description of sequence: DNA for Full VL
  source 1..321: mol_type = other DNA
gatatccagatgacacagactacatcctccctgtctgcctctctgggagacagagtcaccatcagttgcagggcaagtcaggacattaacaattatttaaactggtatcagcagaaaccagatggaactgttaaactcctgatctactacacatcaagatttcactcaggagtcccatcaaggttcagtggcagtgggtctggaacagattattctctcaccattagcaacctggagcaagaagatattgccacttacttttgccaacagggtaatacgcttccgtggacgttcggtggaggcaccaagctggacatcaaa

SEQ ID NO: 53 - protein, 8 aa, synthetic construct
  REGION 1..8: note = Description of sequence: VH CDR1
GFTFSNYA

SEQ ID NO: 54 - protein, 8 aa, synthetic construct
  REGION 1..8: note = Description of sequence: VH CDR2
IGTSGSYT

SEQ ID NO: 55 - protein, 14 aa, synthetic construct
  REGION 1..14: note = Description of sequence: VH CDR3
ARRGYYNPTHAMDY

SEQ ID NO: 56 - protein, 9 aa, synthetic construct
  REGION 1..9: note = Description of sequence: VL CDR1
TGAVTTSNY

SEQ ID NO: 57 - skipped

SEQ ID NO: 58 - protein, 9 aa, synthetic construct
  REGION 1..9: note = Description of sequence: VL CDR3
ALWYSNHLV

SEQ ID NO: 59 - protein, 121 aa, synthetic construct
  REGION 1..121: note = Description of sequence: Full VH
EVMLVESGGALVKPGGSLKLSCAASGFTFSNYAMSWVRQTPEKRLEWVATIGTSGSYTYYSDSVKGRFTISRDNARNTLYLQMTSLRSEDTAMYYCARRGYYNPTHAMDYWGQGTSVTVSS

SEQ ID NO: 60 - protein, 109 aa, synthetic construct
  REGION 1..109: note = Description of sequence: Full VL
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHLVFGGGTKLTVL

SEQ ID NO: 61 - DNA, 363 bp, synthetic construct
  misc_feature 1..363: note = Description of sequence: DNA for Full VH
  source 1..363: mol_type = other DNA
gaggtgatgctggtggagtctgggggagccttagtgaagcctggagggtccctgaaactctcctgtgcagcctctggattcactttcagtaactatgccatgtcttgggttcgccagactccagagaagaggctggagtgggtcgcaaccattggtactagtggtagttacacctactattcagacagtgtgaagggtcgattcaccatctccagagacaatgccaggaacaccctgtacctgcaaatgaccagtctgaggtctgaggacacggccatgtattactgtgcaagacgggggtactacaatccgacccatgctatggactactggggtcaaggaacctcagtcaccgtctcctca

SEQ ID NO: 62 - DNA, 328 bp, synthetic construct
  misc_feature 1..328: note = Description of sequence: DNA for Full VL
  source 1..328: mol_type = other DNA
caggctgttgtgactcaggaatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccatttggtgttcggtggaggaaccaaactgactgtcctag

SEQ ID NO: 63 - protein, 8 aa, synthetic construct
  REGION 1..8: note = Description of sequence: VH CDR1
GYTFSTYW

SEQ ID NO: 64 - protein, 8 aa, synthetic construct
  REGION 1..8: note = Description of sequence: VH CDR2
ILPGSGST

SEQ ID NO: 65 - protein, 14 aa, synthetic construct
  REGION 1..14: note = Description of sequence: VH CDR3
ARGGLYYYGGAMDY

SEQ ID NO: 66 - protein, 12 aa, synthetic construct
  REGION 1..12: note = Description of sequence: VL CDR1
QSLLYSGNQKNY

SEQ ID NO: 67 - protein, 9 aa, synthetic construct
  REGION 1..9: note = Description of sequence: VL CDR3
QQYYSYPYT

SEQ ID NO: 68 - protein, 121 aa, synthetic construct
  REGION 1..121: note = Description of sequence: Full VH
QVQLQQSGAELMKPGASVKISCKATGYTFSTYWIEWVKQRPGHGLEWIGEILPGSGSTNYNENFKGKATFTADTSSNTAYMQLSSLTSEDSAVYFCARGGLYYYGGAMDYWGQGTSVTVSS

SEQ ID NO: 69 - protein, 113 aa, synthetic construct
  REGION 1..113: note = Description of sequence: Full VL
DIVMSQSPSSLAVSVGEKVTMSCKSSQSLLYSGNQKNYLAWYQQKPGQSPKLLIYWASIRESGVPDRFTGSGSGTDFTLTISSVKAEDLAVYYCQQYYSYPYTFGGGTKVEIK

SEQ ID NO: 70 - DNA, 363 bp, synthetic construct
  misc_feature 1..363: note = Description of sequence: DNA for Full VH
  source 1..363: mol_type = other DNA
caggttcagctgcagcagtctggagctgagctgatgaagcctggggcctcagtgaagatatcctgcaaggctactggctacacattcagtacctactggatagagtgggtaaagcagaggcctggacatggccttgagtggattggagagattttacctggaagtggtagtactaactacaatgagaacttcaagggcaaggccacattcactgcagatacatcctccaacacagcctacatgcaactcagcagcctgacatctgaggactctgccgtctatttctgtgctagagggggactttattactacggaggggctatggactactggggtcaaggaacctcagtcaccgtctcctca

SEQ ID NO: 71 - DNA, 339 bp, synthetic construct
  misc_feature 1..339: note = Description of sequence: DNA for Full VL
  source 1..339: mol_type = other DNA
gacattgtgatgtcacagtctccatcctccctcgctgtgtcagttggagagaaggttactatgagctgcaagtccagtcagagccttttatatagtggcaatcaaaagaactacttggcctggtaccagcagaaaccagggcagtctcctaaactgctgatttactgggcatccattagggaatctggggtccctgatcgcttcacaggcagtggatctgggacagatttcactctcaccatcagcagtgtgaaggctgaagacctggcagtttattactgtcagcaatattatagctatccgtacacgttcggaggggggaccaaggtggaaataaaa

SEQ ID NO: 72 - protein, 8 aa, synthetic construct
  REGION 1..8: note = Description of sequence: VH CDR1
GFNIKDTY

SEQ ID NO: 73 - protein, 8 aa, synthetic construct
  REGION 1..8: note = Description of sequence: VH CDR2
IDPANGNT

SEQ ID NO: 74 - protein, 16 aa, synthetic construct
  REGION 1..16: note = Description of sequence: VH CDR3
ARDYYGTIFPYWYFDV

SEQ ID NO: 75 - protein, 5 aa, synthetic construct
  REGION 1..5: note = Description of sequence: VL CDR1
SSVSY

SEQ ID NO: 76 - skipped

SEQ ID NO: 77 - protein, 9 aa, synthetic construct
  REGION 1..9: note = Description of sequence: VL CDR3
QQRSSYPLT

SEQ ID NO: 78 - protein, 123 aa, synthetic construct
  REGION 1..123: note = Description of sequence: Full VH
EVQLQQSGAVLVKPGASVRLSCTASGFNIKDTYIHWVKQRPEQGLEWIGKIDPANGNTKFDPKFQGKATIAADTSSNTAYLQLSSLTSEDTAVYYCARDYYGTIFPYWYFDVWGAGTTVTVSS

SEQ ID NO: 79 - protein, 106 aa, synthetic construct
  REGION 1..106: note = Description of sequence: Full VL
QIVLTQSPAIMSASPGEKVTITCSASSSVSYMHWFQQKPGTSPKFWIYITSNLASGVPARFSGSGSGTSYSLTISRMEAEDAATYYCQQRSSYPLTFGAGTKLELK

SEQ ID NO: 80 - DNA, 369 bp, synthetic construct
  misc_feature 1..369: note = Description of sequence: DNA for Full VH
  source 1..369: mol_type = other DNA
gaggttcagctgcagcagtctggggcagtacttgtgaagccaggggcctcagtcaggttgtcctgtacagcttctggcttcaacattaaagacacctatatacactgggtgaagcagaggcctgaacagggcctggagtggattggaaagattgatcctgcgaatggtaatactaaatttgacccgaagttccagggcaaggccactatagcagcagacacatcctccaacacagcctacctgcagctcagcagcctgacatctgaggacactgccgtctattactgtgctagagattactacggtactatcttcccttactggtacttcgatgtctggggcgcagggaccacggtcaccgtctcctca

SEQ ID NO: 81 - DNA, 318 bp, synthetic construct
  misc_feature 1..318: note = Description of sequence: DNA for Full VL
  source 1..318: mol_type = other DNA
caaattgttctcacccagtctccagcaatcatgtctgcatctccaggggagaaggtcaccataacctgcagtgccagctcaagtgtaagttacatgcactggttccagcagaagccaggcacttctcccaaattctggatttatatcacatccaacctggcttctggagtccctgctcgcttcagtggcagtggatctgggacctcttactctctcacaatcagccgaatggaggctgaagatgctgccacttattactgccagcaaaggagtagttacccactcacgttcggtgctgggaccaagctggagctgaaa

SEQ ID NO: 82 - protein, 15 aa, synthetic construct
  REGION 1..15: note = Description of sequence: VH CDR3
ARSYYTYDQGSWLAY

SEQ ID NO: 83 - protein, 122 aa, synthetic construct
  REGION 1..122: note = Description of sequence: Full VH
QVQLQQSGAELMKPGASVKISCKATGYTFSTYWIEWVKQRPGHGLEWIGEILPGSGSTNYNEKFRGKATFTADTSSNTAYMQLSSLTSEDSAVYYCARSYYTYDQGSWLAYWGQGTLVTVSA

SEQ ID NO: 84 - protein, 113 aa, synthetic construct
  REGION 1..113: note = Description of sequence: Full VL
DIVMSQSPSSLAVSVGEKVTMTCKSSQSLLYSGNQKNYLAWYQQKPGQSPKLLINWASTRESGVPDRLTGSGSGTDFSLTISSVKAEDLAVYYCQQYYSYPYTFGGGTKLEIK

SEQ ID NO: 85 - DNA, 366 bp, synthetic construct
  misc_feature 1..366: note = Description of sequence: DNA for Full VH
  source 1..366: mol_type = other DNA
caggttcagctgcagcagtctggagctgagctgatgaagcctggggcctcagtgaagatatcctgcaaggctactggctacacattcagtacctactggatagagtgggtaaagcagaggcctggacatggccttgagtggattggagagattttacctggaagtggtagtactaactacaatgagaagttcaggggcaaggccacattcactgcagatacatcctccaacacagcctacatgcaactcagcagcctgacatctgaggactctgccgtctattactgtgcaagatcctactatacgtacgaccaggggtcctggcttgcttactggggccaagggactctggtcactgtctctgca

SEQ ID NO: 86 - DNA, 339 bp, synthetic construct
  misc_feature 1..339: note = Description of sequence: DNA for Full VL
  source 1..339: mol_type = other DNA
gacattgtgatgtcacagtctccatcctccctagctgtgtcagttggagagaaggttactatgacctgcaagtccagtcagagccttttatatagtggcaatcaaaagaactacttggcctggtaccagcagaaaccagggcagtctcctaaactgctgattaactgggcatctactagggaatctggggtccctgatcgcttaacaggcagtggatctgggacagatttcagtctcaccatcagcagtgtgaaggctgaagacctggcagtttattactgtcagcaatattatagctatccgtacacgttcggaggggggaccaagctggaaataaaa

SEQ ID NO: 87 - protein, 8 aa, synthetic construct
  REGION 1..8: note = Description of sequence: VH CDR1
GFTFATSW

SEQ ID NO: 88 - protein, 8 aa, synthetic construct
  REGION 1..8: note = Description of sequence: VH CDR2
IHPNSGNT

SEQ ID NO: 89 - protein, 10 aa, synthetic construct
  REGION 1..10: note = Description of sequence: VH CDR3
ARGADYAMDY

SEQ ID NO: 90 - protein, 11 aa, synthetic construct
  REGION 1..11: note = Description of sequence: VL CDR1
QSIVHSNGNTY

SEQ ID NO: 91 - skipped

SEQ ID NO: 92 - protein, 9 aa, synthetic construct
  REGION 1..9: note = Description of sequence: VL CDR3
FQGSHVPWT

SEQ ID NO: 93 - protein, 117 aa, synthetic construct
  REGION 1..117: note = Description of sequence: Full VH
QVQLQQPGSVLVRPGASVKLSCKASGFTFATSWMHWAKERPGQGLEWIGQIHPNSGNTNYNEKFKGKATLTVDTSSSTAYVDLSSLTSEDSAVYYCARGADYAMDYWGQGTSVTVSS

SEQ ID NO: 94 - protein, 112 aa, synthetic construct
  REGION 1..112: note = Description of sequence: Full VL
DVLMTQTPLSLPVSLGDQASISCRSSQSIVHSNGNTYLEWYLQKPGQSPKLLIYIVSNRFSGVPDRFSGSGSGTDFTLKISRVEAEDLGVYYCFQGSHVPWTFGGGTKLEIK

SEQ ID NO: 95 - DNA, 351 bp, synthetic construct
  misc_feature 1..351: note = Description of sequence: DNA for Full VH
  source 1..351: mol_type = other DNA
caggtccaactgcagcagcctgggtctgtgctggtgaggcctggagcttcagtgaagctgtcctgcaaggcttctggcttcaccttcgccacctcctggatgcactgggcgaaggagaggcctggacaaggccttgagtggattggacagattcatcctaatagtggtaatactaactacaatgagaagttcaagggcaaggccacactgactgtagacacatcctccagcacagcctacgtggatctcagcagcctgacatctgaggactctgcggtctattactgtgcaagaggggcggactatgctatggactactggggtcaaggaacctcagtcaccgtctcctca

SEQ ID NO: 96 - DNA, 336 bp, synthetic construct
  misc_feature 1..336: note = Description of sequence: DNA for Full VL
  source 1..336: mol_type = other DNA
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacatagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 97 - protein, 220 aa, synthetic construct
  REGION 1..220: note = Description of sequence: CD28 polypeptide component
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 98 - protein, 164 aa, synthetic construct
  REGION 1..164: note = Description of sequence: intracellular signaling domain
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 99 - protein, 22 aa, synthetic construct
  REGION 1..22: note = Description of sequence: Native ITAM1
QNQLYNELNLGRREEYDVLDKR

SEQ ID NO: 100 - DNA, 66 bp, synthetic construct
  misc_feature 1..66: note = Description of sequence: Native ITAM1 coding sequence
  source 1..66: mol_type = other DNA
cagaaccagctctataacgagctcaatctaggacgaagagaggagtacgatgttttggacaagaga

SEQ ID NO: 101 - protein, 22 aa, synthetic construct
  REGION 1..22: note = Description of sequence: ITAM1 variant
QNQLFNELNLGRREEFDVLDKR

SEQ ID NO: 102 - DNA, 66 bp, synthetic construct
  misc_feature 1..66: note = Description of sequence: ITAM1 variant coding sequence
  source 1..66: mol_type = other DNA
cagaaccagctctttaacgagctcaatctaggacgaagagaggagttcgatgttttggacaagaga

SEQ ID NO: 103 - protein, 23 aa, synthetic construct
  REGION 1..23: note = Description of sequence: native ITAM2
QEGLYNELQKDKMAEAYSEIGMK

SEQ ID NO: 104 - DNA, 69 bp, synthetic construct
  misc_feature 1..69: note = Description of sequence: native ITAM2 coding sequence
  source 1..69: mol_type = other DNA
caggaaggcctgtacaatgaactgcagaaagataagatggcggaggcctacagtgagattgggatgaaa

SEQ ID NO: 105 - protein, 23 aa, synthetic construct
  REGION 1..23: note = Description of sequence: ITAM2 variant
QEGLFNELQKDKMAEAFSEIGMK

SEQ ID NO: 106 - DNA, 69 bp, synthetic construct
  misc_feature 1..69: note = Description of sequence: ITAM2 variant coding sequence
  source 1..69: mol_type = other DNA
caggaaggcctgttcaatgaactgcagaaagataagatggcggaggccttcagtgagattgggatgaaa

SEQ ID NO: 107 - protein, 22 aa, synthetic construct
  REGION 1..22: note = Description of sequence: native ITAM3
HDGLYQGLSTATKDTYDALHMQ

SEQ ID NO: 108 - DNA, 66 bp, synthetic construct
  misc_feature 1..66: note = Description of sequence: native ITAM3 coding sequence
  source 1..66: mol_type = other DNA
cacgatggcctttaccagggtctcagtacagccaccaaggacacctacgacgcccttcacatgcag

SEQ ID NO: 109 - protein, 22 aa, synthetic construct
  REGION 1..22: note = Description of sequence: ITAM3 variant
HDGLFQGLSTATKDTFDALHMQ

SEQ ID NO: 110 - DNA, 66 bp, synthetic construct
  misc_feature 1..66: note = Description of sequence: ITAM3 variant coding sequence
  source 1..66: mol_type = other DNA
cacgatggccttttccaggggctcagtacagccaccaaggacaccttcgacgcccttcacatgcag

SEQ ID NO: 111 - protein, 112 aa, synthetic construct
  REGION 1..112: note = Description of sequence: intracellular signaling domain
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 112 - DNA, 336 bp, synthetic construct
  misc_feature 1..336: note = Description of sequence: intracellular signaling domain coding sequence
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 113 - DNA, 123 bp, synthetic construct
  misc_feature 1..123: note = Description of sequence: intracellular signaling domain coding sequence
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 114 - protein, 218 aa, synthetic construct
  REGION 1..218: note = Description of sequence: intracellular signaling domain
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 115 - protein, 255 aa, synthetic construct
  REGION 1..255: note = Description of sequence: intracellular signaling domain
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 116 - protein, 20 aa, synthetic construct
  REGION 1..20: note = Description of sequence: signal sequence
METDTLLLWVLLLWVPGSTG

SEQ ID NO: 117 - DNA, 60 bp, synthetic construct
  misc_feature 1..60: note = Description of sequence: signal sequence coding sequence
  source 1..60: mol_type = other DNA
atggaaaccgatacactcttgctgtgggtgcttcttctttgggtgcctggatctactggc

SEQ ID NO: 118 - DNA, 357 bp, synthetic construct
  misc_feature 1..357: note = Description of sequence: DNA for Full VH
  source 1..357: mol_type = other DNA
gaagttcaactggttgagtctggaggtgatttggttaagcctggtggatctttgaagctttcatgtgctgctagcggctttacgttttccagctatggaatgtcatgggtgagacagaccccggacaagaggctcgagtgggttgcgactatatcaagcgatggaagtcacacctactatcctgattccgttaaagggagattcactatctccagggacatcgccaagaacaccctgtatctgcagatgtcctctctcaaatcagaggatactgcgatgtattattgcgtgcgccaagggagatatgtaccatctttcgacgtatggggcgcaggcaccaccgtcaccgtaagcagc

SEQ ID NO: 119 - DNA, 321 bp, synthetic construct
  misc_feature 1..321: note = Description of sequence: DNA for Full VL
  source 1..321: mol_type = other DNA
gatattgtcctgacgcaaagtccggcgaccctgtcagttacgcccggagacagcgtttctttgtcatgtagagctagtcagtcaattcgaaataatcttcattggtatcaacaaaagagccacgagtctccgcgcttgttgatcaaagacgcttcccagtcaataagtggtaccccgagtcgcttctctggttccggaagtggcacagactttacgctttctatcaactccgtagagactgaggatttcggcatgtatttttgccatcaaacgaataattggcctttcacgtttggatctggtactaaactggaaataaag

SEQ ID NO: 120 - DNA, 354 bp, synthetic construct
  misc_feature 1..354: note = Description of sequence: DNA for Full VH
  source 1..354: mol_type = other DNA
caggtacagctgcagcaaccaggtgccgaattggtcaaacccggtgcaagcgtaaaacttagttgtaaagcgagcggctacacattcatcaattattggatcaactggatgcgacagcgcccaggtcagggacttgaatggataggtaatatttatccgggttcctcaagtacaacgtacaacgagaaatttaagtcaaaagctgcattgaccgtggatacgtccagctccactgcgtacatgcagctcagttcacttacgtctgatgattccgccgtatatttctgcgctcggagcgtgggttactggtactttgacgtatggggggctggaaccacagttacggttagtagc

SEQ ID NO: 121 - DNA, 339 bp, synthetic construct
  misc_feature 1..339: note = Description of sequence: DNA for Full VL
  source 1..339: mol_type = other DNA
gacatagttatgtcacagtctccatcctcacttgccgtctcagtcggcgaaaaggtaacactgagttgcaagtctagccagagtctttttttttcaggtaaccaaaaaaactttcttgcatggtaccagcagaagccaggacagtcaccgaacctcctgatattttgggcaagcacgcgcgagtcaggtgtaccggacaggttcaccggtagtggcagcggaactgatttcacactcacgataagttccgtgaaggctgaggatttggctgtgtactattgccaacaatactatagctatcctttcacgtttggttctggcacgaagctggaaatcaag

SEQ ID NO: 122 - protein, 326 aa, synthetic construct
  REGION 1..326: note = Description of sequence: Fc fusion protein
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 123 - DNA, 978 bp, synthetic construct
  misc_feature 1..978: note = Description of sequence: Fc fusion protein coding sequence
  source 1..978: mol_type = other DNA
gccagcaccaaaggaccctccgttttcccacttgccccctccagtagaagcacttcagaaagcaccgcggcgcttgggtgtctggttaaagattattttcctgaacccgttaccgtctcctggaacagtggggcgcttacatctggtgtgcacacattcccagcagtactccagtcatcaggactgtactcattgagctccgttgttactgttcccagctccaactttggaacccaaacctacacctgcaacgtcgatcataagccgagcaatacaaaggtagataaaacagtggagcgaaaatgttgtgtcgaatgccctccgtgcccggctccccctgttgctggcccgtcagtctttctgtttccaccaaaacccaaagacactctcatgatcagcagaacacctgaggtaacttgcgtcgtggtagacgtttcacacgaagacccagaagtccaattcaattggtacgtcgatggggttgaggtccacaacgccaaaacaaagccccgagaagagcagtttaacagcacttttagagtggttagtgtattgacggtcgtgcaccaagactggctcaacggaaaggaatataaatgtaaagtatcaaacaaaggtctcccagcaccaattgaaaaaacaattagcaagacaaaaggacaaccgcgagaaccgcaggtctacactctgcctccttcaagggaggagatgactaagaatcaagtttctcttacctgtctcgtaaaaggattctacccctctgatatagcggtggagtgggagtccaacggacagcccgaaaacaactacaagacaacccccccaatgctcgactcagacgggtcttttttcctttattccaagttgacagtcgataagtcaagatggcaacaggggaatgttttctcatgctccgtaatgcacgaagcgttgcataaccactatacgcaaaaatctctgagtctttcaccgggcaag

SEQ ID NO: 124 - protein, 228 aa, synthetic construct
  REGION 1..228: note = Description of sequence: Fc fusion protein
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 125 - DNA, 684 bp, synthetic construct
  misc_feature 1..684: note = Description of sequence: Fc fusion protein coding sequence
  source 1..684: mol_type = other DNA
gagcgaaaatgttgtgtcgaatgccctccgtgcccggctccccctgttgctggcccgtcagtctttctgtttccaccaaaacccaaagacactctcatgatcagcagaacacctgaggtaacttgcgtcgtggtagacgtttcacacgaagacccagaagtccaattcaattggtacgtcgatggggttgaggtccacaacgccaaaacaaagccccgagaagagcagtttaacagcacttttagagtggttagtgtattgacggtcgtgcaccaagactggctcaacggaaaggaatataaatgtaaagtatcaaacaaaggtctcccagcaccaattgaaaaaacaattagcaagacaaaaggacaaccgcgagaaccgcaggtctacactctgcctccttcaagggaggagatgactaagaatcaagtttctcttacctgtctcgtaaaaggattctacccctctgatatagcggtggagtgggagtccaacggacagcccgaaaacaactacaagacaacccccccaatgctcgactcagacgggtcttttttcctttattccaagttgacagtcgataagtcaagatggcaacaggggaatgttttctcatgctccgtaatgcacgaagcgttgcataaccactatacgcaaaaatctctgagtctttcaccgggcaag

SEQ ID NO: 126 - DNA, 45 bp, synthetic construct
  misc_feature 1..45: note = Description of sequence: linker coding sequence
  source 1..45: mol_type = other DNA
ggcggcggaggtagtggaggaggcggttcaggtggagggggatct

SEQ ID NO: 127 - protein, 9 aa, synthetic construct
  REGION 1..9: note = Description of sequence: HA Tag
YPYDVPDYA

SEQ ID NO: 128 - DNA, 27 bp, synthetic construct
  misc_feature 1..27: note = Description of sequence: HA Tag coding sequence
  source 1..27: mol_type = other DNA
tacccatacgacgtaccagattacgct

SEQ ID NO: 129 - protein, 611 aa, synthetic construct
  REGION 1..611: note = Description of sequence: Fc fusion protein
METDTLLLWVLLLWVPGSTGEVQLVESGGDLVKPGGSLKLSCAASGFTFSSYGMSWVRQTPDKRLEWVATISSDGSHTYYPDSVKGRFTISRDIAKNTLYLQMSSLKSEDTAMYYCVRQGRYVPSFDVWGAGTTVTVSSGGGGSGGGGSGGGGSDIVLTQSPATLSVTPGDSVSLSCRASQSIRNNLHWYQQKSHESPRLLIKDASQSISGTPSRFSGSGSGTDFTLSINSVETEDFGMYFCHQTNNWPFTFGSGTKLEIKGGGGSGGGGSGGGGSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKYPYDVPDYA

SEQ ID NO: 130 - DNA, 1854 bp, synthetic construct
  misc_feature 1..1854: note = Description of sequence: Fc fusion protein coding sequence
  source 1..1854: mol_type = other DNA
atggaaaccgatacactcttgctgtgggtgcttcttctttgggtgcctggatctactggcgaagttcaactggttgagtctggaggtgatttggttaagcctggtggatctttgaagctttcatgtgctgctagcggctttacgttttccagctatggaatgtcatgggtgagacagaccccggacaagaggctcgagtgggttgcgactatatcaagcgatggaagtcacacctactatcctgattccgttaaagggagattcactatctccagggacatcgccaagaacaccctgtatctgcagatgtcctctctcaaatcagaggatactgcgatgtattattgcgtgcgccaagggagatatgtaccatctttcgacgtatggggcgcaggcaccaccgtcaccgtaagcagcggcggcggaggtagtggaggaggcggttcaggtggagggggatctgatattgtcctgacgcaaagtccggcgaccctgtcagttacgcccggagacagcgtttctttgtcatgtagagctagtcagtcaattcgaaataatcttcattggtatcaacaaaagagccacgagtctccgcgcttgttgatcaaagacgcttcccagtcaataagtggtaccccgagtcgcttctctggttccggaagtggcacagactttacgctttctatcaactccgtagagactgaggatttcggcatgtatttttgccatcaaacgaataattggcctttcacgtttggatctggtactaaactggaaataaagggaggtggcggaagtggtgggggaggatcagggggtggaggttctgccagcaccaaaggaccctccgttttcccacttgccccctccagtagaagcacttcagaaagcaccgcggcgcttgggtgtctggttaaagattattttcctgaacccgttaccgtctcctggaacagtggggcgcttacatctggtgtgcacacattcccagcagtactccagtcatcaggactgtactcattgagctccgttgttactgttcccagctccaactttggaacccaaacctacacctgcaacgtcgatcataagccgagcaatacaaaggtagataaaacagtggagcgaaaatgttgtgtcgaatgccctccgtgcccggctccccctgttgctggcccgtcagtctttctgtttccaccaaaacccaaagacactctcatgatcagcagaacacctgaggtaacttgcgtcgtggtagacgtttcacacgaagacccagaagtccaattcaattggtacgtcgatggggttgaggtccacaacgccaaaacaaagccccgagaagagcagtttaacagcacttttagagtggttagtgtattgacggtcgtgcaccaagactggctcaacggaaaggaatataaatgtaaagtatcaaacaaaggtctcccagcaccaattgaaaaaacaattagcaagacaaaaggacaaccgcgagaaccgcaggtctacactctgcctccttcaagggaggagatgactaagaatcaagtttctcttacctgtctcgtaaaaggattctacccctctgatatagcggtggagtgggagtccaacggacagcccgaaaacaactacaagacaacccccccaatgctcgactcagacgggtcttttttcctttattccaagttgacagtcgataagtcaagatggcaacaggggaatgttttctcatgctccgtaatgcacgaagcgttgcataaccactatacgcaaaaatctctgagtctttcaccgggcaagtacccatacgacgtaccagattacgctgcgatcgcagatccggattag

SEQ ID NO: 131 - protein, 513 aa, synthetic construct
  REGION 1..513: note = Description of sequence: FC Fusion peptide
METDTLLLWVLLLWVPGSTGEVQLVESGGDLVKPGGSLKLSCAASGFTFSSYGMSWVRQTPDKRLEWVATISSDGSHTYYPDSVKGRFTISRDIAKNTLYLQMSSLKSEDTAMYYCVRQGRYVPSFDVWGAGTTVTVSSGGGGSGGGGSGGGGSDIVLTQSPATLSVTPGDSVSLSCRASQSIRNNLHWYQQKSHESPRLLIKDASQSISGTPSRFSGSGSGTDFTLSINSVETEDFGMYFCHQTNNWPFTFGSGTKLEIKGGGGSGGGGSGGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKYPYDVPDYA

SEQ ID NO: 132 - DNA, 1560 bp, synthetic construct
  misc_feature 1..1560: note = Description of sequence: Fc fusion protein coding sequence
  source 1..1560: mol_type = other DNA
atggaaaccgatacactcttgctgtgggtgcttcttctttgggtgcctggatctactggcgaagttcaactggttgagtctggaggtgatttggttaagcctggtggatctttgaagctttcatgtgctgctagcggctttacgttttccagctatggaatgtcatgggtgagacagaccccggacaagaggctcgagtgggttgcgactatatcaagcgatggaagtcacacctactatcctgattccgttaaagggagattcactatctccagggacatcgccaagaacaccctgtatctgcagatgtcctctctcaaatcagaggatactgcgatgtattattgcgtgcgccaagggagatatgtaccatctttcgacgtatggggcgcaggcaccaccgtcaccgtaagcagcggcggcggaggtagtggaggaggcggttcaggtggagggggatctgatattgtcctgacgcaaagtccggcgaccctgtcagttacgcccggagacagcgtttctttgtcatgtagagctagtcagtcaattcgaaataatcttcattggtatcaacaaaagagccacgagtctccgcgcttgttgatcaaagacgcttcccagtcaataagtggtaccccgagtcgcttctctggttccggaagtggcacagactttacgctttctatcaactccgtagagactgaggatttcggcatgtatttttgccatcaaacgaataattggcctttcacgtttggatctggtactaaactggaaataaagggaggtggcggaagtggtgggggaggatcagggggtggaggttctgagcgaaaatgttgtgtcgaatgccctccgtgcccggctccccctgttgctggcccgtcagtctttctgtttccaccaaaacccaaagacactctcatgatcagcagaacacctgaggtaacttgcgtcgtggtagacgtttcacacgaagacccagaagtccaattcaattggtacgtcgatggggttgaggtccacaacgccaaaacaaagccccgagaagagcagtttaacagcacttttagagtggttagtgtattgacggtcgtgcaccaagactggctcaacggaaaggaatataaatgtaaagtatcaaacaaaggtctcccagcaccaattgaaaaaacaattagcaagacaaaaggacaaccgcgagaaccgcaggtctacactctgcctccttcaagggaggagatgactaagaatcaagtttctcttacctgtctcgtaaaaggattctacccctctgatatagcggtggagtgggagtccaacggacagcccgaaaacaactacaagacaacccccccaatgctcgactcagacgggtcttttttcctttattccaagttgacagtcgataagtcaagatggcaacaggggaatgttttctcatgctccgtaatgcacgaagcgttgcataaccactatacgcaaaaatctctgagtctttcaccgggcaagtacccatacgacgtaccagattacgctgcgatcgcagatccggattag

SEQ ID NO: 133 - protein, 616 aa, synthetic construct
  REGION 1..616: note = Description of sequence: Fc fusion protein
METDTLLLWVLLLWVPGSTGQVQLQQPGAELVKPGASVKLSCKASGYTFINYWINWMRQRPGQGLEWIGNIYPGSSSTTYNEKFKSKAALTVDTSSSTAYMQLSSLTSDDSAVYFCARSVGYWYFDVWGAGTTVTVSSGGGGSGGGGSGGGGSDIVMSQSPSSLAVSVGEKVTLSCKSSQSLFFSGNQKNFLAWYQQKPGQSPNLLIFWASTRESGVPDRFTGSGSGTDFTLTISSVKAEDLAVYYCQQYYSYPFTFGSGTKLEIKGGGGSGGGGSGGGGSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKYPYDVPDYA

SEQ ID NO: 134 - DNA, 1869 bp, synthetic construct
  misc_feature 1..1869: note = Description of sequence: Fc fusion protein coding sequence
  source 1..1869: mol_type = other DNA
atggaaaccgatacactcttgctgtgggtgcttcttctttgggtgcctggatctactggccaggtacagctgcagcaaccaggtgccgaattggtcaaacccggtgcaagcgtaaaacttagttgtaaagcgagcggctacacattcatcaattattggatcaactggatgcgacagcgcccaggtcagggacttgaatggataggtaatatttatccgggttcctcaagtacaacgtacaacgagaaatttaagtcaaaagctgcattgaccgtggatacgtccagctccactgcgtacatgcagctcagttcacttacgtctgatgattccgccgtatatttctgcgctcggagcgtgggttactggtactttgacgtatggggggctggaaccacagttacggttagtagcggcggcggaggtagtggaggaggcggttcaggtggagggggatctgacatagttatgtcacagtctccatcctcacttgccgtctcagtcggcgaaaaggtaacactgagttgcaagtctagccagagtctttttttttcaggtaaccaaaaaaactttcttgcatggtaccagcagaagccaggacagtcaccgaacctcctgatattttgggcaagcacgcgcgagtcaggtgtaccggacaggttcaccggtagtggcagcggaactgatttcacactcacgataagttccgtgaaggctgaggatttggctgtgtactattgccaacaatactatagctatcctttcacgtttggttctggcacgaagctggaaatcaagggaggtggcggaagtggtgggggaggatcagggggtggaggttctgccagcaccaaaggaccctccgttttcccacttgccccctccagtagaagcacttcagaaagcaccgcggcgcttgggtgtctggttaaagattattttcctgaacccgttaccgtctcctggaacagtggggcgcttacatctggtgtgcacacattcccagcagtactccagtcatcaggactgtactcattgagctccgttgttactgttcccagctccaactttggaacccaaacctacacctgcaacgtcgatcataagccgagcaatacaaaggtagataaaacagtggagcgaaaatgttgtgtcgaatgccctccgtgcccggctccccctgttgctggcccgtcagtctttctgtttccaccaaaacccaaagacactctcatgatcagcagaacacctgaggtaacttgcgtcgtggtagacgtttcacacgaagacccagaagtccaattcaattggtacgtcgatggggttgaggtccacaacgccaaaacaaagccccgagaagagcagtttaacagcacttttagagtggttagtgtattgacggtcgtgcaccaagactggctcaacggaaaggaatataaatgtaaagtatcaaacaaaggtctcccagcaccaattgaaaaaacaattagcaagacaaaaggacaaccgcgagaaccgcaggtctacactctgcctccttcaagggaggagatgactaagaatcaagtttctcttacctgtctcgtaaaaggattctacccctctgatatagcggtggagtgggagtccaacggacagcccgaaaacaactacaagacaacccccccaatgctcgactcagacgggtcttttttcctttattccaagttgacagtcgataagtcaagatggcaacaggggaatgttttctcatgctccgtaatgcacgaagcgttgcataaccactatacgcaaaaatctctgagtctttcaccgggcaagtacccatacgacgtaccagattacgctgcgatcgcagatccggattag

SEQ ID NO: 135 - protein, 518 aa, synthetic construct
  REGION 1..518: note = Description of sequence: Fc fusion protein
METDTLLLWVLLLWVPGSTGQVQLQQPGAELVKPGASVKLSCKASGYTFINYWINWMRQRPGQGLEWIGNIYPGSSSTTYNEKFKSKAALTVDTSSSTAYMQLSSLTSDDSAVYFCARSVGYWYFDVWGAGTTVTVSSGGGGSGGGGSGGGGSDIVMSQSPSSLAVSVGEKVTLSCKSSQSLFFSGNQKNFLAWYQQKPGQSPNLLIFWASTRESGVPDRFTGSGSGTDFTLTISSVKAEDLAVYYCQQYYSYPFTFGSGTKLEIKGGGGSGGGGSGGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKYPYDVPDYA

SEQ ID NO: 136 - DNA, 1575 bp, synthetic construct
  misc_feature 1..1575: note = Description of sequence: Fc fusion protein coding sequence
  source 1..1575: mol_type = other DNA
atggaaaccgatacactcttgctgtgggtgcttcttctttgggtgcctggatctactggccaggtacagctgcagcaaccaggtgccgaattggtcaaacccggtgcaagcgtaaaacttagttgtaaagcgagcggctacacattcatcaattattggatcaactggatgcgacagcgcccaggtcagggacttgaatggataggtaatatttatccgggttcctcaagtacaacgtacaacgagaaatttaagtcaaaagctgcattgaccgtggatacgtccagctccactgcgtacatgcagctcagttcacttacgtctgatgattccgccgtatatttctgcgctcggagcgtgggttactggtactttgacgtatggggggctggaaccacagttacggttagtagcggcggcggaggtagtggaggaggcggttcaggtggagggggatctgacatagttatgtcacagtctccatcctcacttgccgtctcagtcggcgaaaaggtaacactgagttgcaagtctagccagagtctttttttttcaggtaaccaaaaaaactttcttgcatggtaccagcagaagccaggacagtcaccgaacctcctgatattttgggcaagcacgcgcgagtcaggtgtaccggacaggttcaccggtagtggcagcggaactgatttcacactcacgataagttccgtgaaggctgaggatttggctgtgtactattgccaacaatactatagctatcctttcacgtttggttctggcacgaagctggaaatcaagggaggtggcggaagtggtgggggaggatcagggggtggaggttctgagcgaaaatgttgtgtcgaatgccctccgtgcccggctccccctgttgctggcccgtcagtctttctgtttccaccaaaacccaaagacactctcatgatcagcagaacacctgaggtaacttgcgtcgtggtagacgtttcacacgaagacccagaagtccaattcaattggtacgtcgatggggttgaggtccacaacgccaaaacaaagccccgagaagagcagtttaacagcacttttagagtggttagtgtattgacggtcgtgcaccaagactggctcaacggaaaggaatataaatgtaaagtatcaaacaaaggtctcccagcaccaattgaaaaaacaattagcaagacaaaaggacaaccgcgagaaccgcaggtctacactctgcctccttcaagggaggagatgactaagaatcaagtttctcttacctgtctcgtaaaaggattctacccctctgatatagcggtggagtgggagtccaacggacagcccgaaaacaactacaagacaacccccccaatgctcgactcagacgggtcttttttcctttattccaagttgacagtcgataagtcaagatggcaacaggggaatgttttctcatgctccgtaatgcacgaagcgttgcataaccactatacgcaaaaatctctgagtctttcaccgggcaagtacccatacgacgtaccagattacgctgcgatcgcagatccggattag

SEQ ID NO: 137 - protein, 122 aa, synthetic construct
  REGION 1..122: note = Description of sequence: VH Full length
QVQLVESGGGVVQPGRSLRLSCAASGFTFSNFGMHWVRQAPGKGLEWVAYITSGGSSINYADTVKGRFTISRDNSKNTLYLQMNSLRAEDTAVYYCTRGGTGTRSLYYFDYWGQGTTVTVSS

SEQ ID NO: 138 - protein, 108 aa, synthetic construct
  REGION 1..108: note = Description of sequence: VL Full Length
DIQMTQSPRSLSASVGDRVTITCRASQDIGNFLNWYQQKPGKAPKLLIYYTSRLQSGVPSRFSGSGSGTDYTLTISSLQPEDFATYYCQQGKTLPYTFGGGTKVEIKD

SEQ ID NO: 139 - protein, 5 aa, synthetic construct
  REGION 1..5: note = Description of sequence: VH CDR1
NFGMH

SEQ ID NO: 140 - protein, 17 aa, synthetic construct
  REGION 1..17: note = Description of sequence: VH CDR2
YITSGGSSINYADTVKG

SEQ ID NO: 141 - protein, 13 aa, synthetic construct
  REGION 1..13: note = Description of sequence: VH CDR3
GGTGTRSLYYFDY

SEQ ID NO: 142 - protein, 11 aa, synthetic construct
  REGION 1..11: note = Description of sequence: VL CDR1
RASQDIGNFLN

SEQ ID NO: 143 - protein, 7 aa, synthetic construct
  REGION 1..7: note = Description of sequence: VL CDR2
YTSRLQS

SEQ ID NO: 144 - protein, 9 aa, synthetic construct
  REGION 1..9: note = Description of sequence: VL CDR3
QQGKTLPYT

SEQ ID NO: 145 - protein, 139 aa, synthetic construct
  REGION 1..139: note = Description of sequence: VH Full Length
MALPVTALLLPLALLLHAEVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVSS

SEQ ID NO: 146 - protein, 110 aa, synthetic construct
  REGION 1..110: note = Description of sequence: VL Full Length
AFQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKAAA

SEQ ID NO: 147 - protein, 10 aa, synthetic construct
  REGION 1..10: note = Description of sequence: VH CDR1
GFNIKDYFLH

SEQ ID NO: 148 - protein, 17 aa, synthetic construct
  REGION 1..17: note = Description of sequence: VH CDR2
WINPDNGNTVYDPKFQG

SEQ ID NO: 149 - protein, 11 aa, synthetic construct
  REGION 1..11: note = Description of sequence: VH CDR3
DYTYEKAALDY

SEQ ID NO: 150 - protein, 11 aa, synthetic construct
  REGION 1..11: note = Description of sequence: VL CDR1
RASGNIYNYLA

SEQ ID NO: 151 - protein, 7 aa, synthetic construct
  REGION 1..7: note = Description of sequence: VL CDR2
DAKTLAD

SEQ ID NO: 152 - protein, 9 aa, synthetic construct
  REGION 1..9: note = Description of sequence: VL CDR3
QHFWSLPFT

SEQ ID NO: 153 - protein, 112 aa, synthetic construct
  REGION 1..112: note = Description of sequence: CD3-Zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR